(19) United States
(12) Patent Application Publication
(10) Pub. No.: US 2025/0210201 A1
(43) Pub. Date: Jun. 26, 2025

(54) METHODS FOR STRATIFICATION OF PARKINSON'S DISEASE PATIENTS, SYSTEMS AND USES THEREOF

(71) Applicant: Université du Luxembourg, Esch-sur-Alzette (LU)

(72) Inventors: Giuseppe ARENA, Audun le Tiche (FR); Patrick MAY, Trier (DE); Rejko KRÜGER, Luxembourg (LU); Anne GRÜNEWALD, Soleuvre (LU); Zied LANDOULSI, Esch-sur-Alzette (LU)

(21) Appl. No.: 18/846,747

(22) PCT Filed: Mar. 15, 2023
(86) PCT No.: PCT/EP2023/056567

§ 371 Date: Sep. 13, 2024

(30) Foreign Application Priority Data
Mar. 15, 2022 (EP) 22162347.3
Aug. 19, 2022 (EP) 22191345.2
Sep. 12, 2022 (LU) LU502780

(51) Int. Cl. G16H 50/30; C12Q 1/6883; G01N 33/50; G16B 20/20

(52) U.S. Cl. CPC G16H 50/30; C12Q 1/6883; G01N 33/502; G01N 33/5073; G16B 20/20

(57) ABSTRACT

The present invention provides for methods for the stratification of Parkinson's disease patients, systems and uses thereof. Provided are methods of diagnosis, uses and methods for screening, compositions and systems.

Polygenic risk score (PRS) is a statistical tool that captures the cumulative effect of multiple single-nucleotide polymorphisms (SNPs) across the human genome to estimate the risk of an individual for a specific disease or trait (DOI: 10.1038/s41591-021-01549-6). Applying PRSs to a restricted number of genes regulating biological functions known to be altered in a particular disease—e.g. mitochondria in Parkinson's disease (PD)—potentially allows to identify pathways involved in the pathological phenotype and stratify patients accordingly for more refined diagnoses and precision medicine therapeutic approaches.

PRSs are usually built from summary statistics of available genome-wide association studies (GWAS) data, calculated as the sum of common low-risk variants with small or moderate effect size. Because of these features, PRS represents a useful prognostic tool especially in multifactorial common disorders, which etiology lies in both polygenic and environmental components.

In Parkinson's disease (PD), which is the second most common neurodegenerative disorder, about 90% of the patients are idiopathic (iPD). However, based on current hypothesis, the genetic contribution to PD risk would be underestimated (the so-called missing heritability), and disease susceptibility may depend more on the synergistic effect of a large number of common low-risk genetic variants (DOI: 10.1016/S1474-4422(19)30320-5).

Thus, in recent years PRS has been extensively used as a risk stratification tool, with the aim to identify individuals with a higher likelihood to develop PD or specific clinical phenotypes (DOI: 10.3390/jpm11101030).

Given the large body of evidence pointing to mitochondrial dysfunction as a major cause of PD pathogenesis, studies on mitochondria-specific PRS could help to define therapeutic strategies aimed at restoring mitochondrial function in specific subgroups (high-mitorisk) of iPD patients.

A recent study conducted by Billingsley and colleagues used a mitochondria-specific PRS to demonstrate that common variants within genes regulating mitochondrial function were significantly associated with higher PD risk (DOI: 10.1038/s41531-019-0080-x).

Of note, PRSs can also be calculated from GWAS common SNPs in subsets of genes implicated in specific subcellular pathways. By using this approach, Paliwal et al. recently developed mitochondrial risk scores (mitoPRSs) for gene sets encoding specific components of different mitochondrial pathways (e.g. oxidative phosphorylation, mitochondrial membrane potential, mitophagy, etc.), demonstrating a significant association of three mitoPRSs with Alzheimer's disease (DOI: 10.1016/j.neurobiolaging.2021.08.005).

PRS used as a standalone tool can be considered as any other risk predictor; it is certainly very useful to improve prediction models, but can also lead to incorrect or imprecise estimates mainly due to “false positive” results (e.g. wrong ranking of individuals as ‘high risk’ because of statistical imprecision) and/or performance bias (e.g. individual not adequately represented by the original study population).

To cope with this, researchers usually evaluate PRS performance also in independent datasets (e.g. different cohorts), but results are sometimes inconsistent. Concomitant assessment of clinical and/or environmental risk factors could be integrated with PRS to improve patient stratification accuracy, but there are still gaps to be filled before translating PRS-based prediction into clinical practice. In particular, functional validation of PRS-based predicted phenotypes in patient-based models is a crucial point that still needs to be addressed to date, especially in the PD field.

Accordingly, the technical problem underling the present invention is to address the needs of the prior art. The technical problem is solved by the embodiments described herein and set out in the claims.

Given that only ˜10% of PD cases can be clearly attributed to monogenic causes, it was hypothesized that a submerged fraction of idiopathic PD cases may harbour a pathogenic combination of common variants in mitochondrial genes ultimately resulting in mitochondrial dysfunction and neurodegeneration. To gain essential knowledge on this mitochondria-related “missing heritability”, the inventors analyzed distribution of mitochondria-specific polygenic risk scores (mitoPRS) in the Luxembourg Parkinson's Study including a PD cohort, and replicated their findings in the larger COURAGE-PD consortium. Starting from available genomic data, they managed to functionally validate mitochondrial polygenic risk profiles in patient-based cellular models established from genetically-stratified Luxembourg Parkinson's Study participants, thus defining mitochondrial pathways potentially involved in neurodegeneration in subgroups of iPD patients.

To capture the cumulative effect of common variants in mitochondrial genes on PD risk, the inventors used the largest summary statistics of PD GWAS to date (Nalls et al., 2019) and common SNPs data from the Luxembourg Parkinson's study (412 iPD patients and 576 healthy controls) and from the COURAGE-PD dataset (10,449 iPD patients and 8,484 healthy controls), and then calculated mitochondria-specific polygenic risk scores (mitoPRS).

Applying the PRS approach to selected mitochondrial pathways, the inventors demonstrated that common variants in genes regulating Oxidative Phosphorylation (OXPHOS) were significantly associated with a higher PD risk in both cohorts. Surprisingly, the association was stronger than in other mitochondrial pathways, such as e.g. proton transport pathway and mitophagy pathway. Primary skin fibroblasts and the corresponding induced pluripotent stem cells (iPSC)-derived neuronal progenitors from Luxembourg Parkinson's Study iPD patients classified based on these mitoPRS, more specifically on their individual OXPHOS-PRS, (high-risk vs low-risk) were then subjected to a comprehensive mitochondrial phenotyping.

Strikingly, the inventors found significant and thus remarkable differences in mitochondrial oxygen consumption rates (OCRs) between the high and low OXPHOS-PRS groups, whereas other phenotypic readouts of mitochondrial function, such as mitophagy and mtDNA levels, remained unchanged.

To study the relevance of common variants in mitochondrial genes for defining stratificable PD progression patterns, the inventors looked at distinct PD-specific clinical phenotypes to identify a potential association with their functionally validated mitoPRSs.

The inventors' functionally validated OXPHOS-specific mitoPRS could thus be used as a genetic tool to stratify the heterogeneous group of iPD patients.

Using patient-based models relying on these mitochondrial signatures for drug screening approaches may pave the way for future more tailored therapeutic strategies.

Moreover, since elevated genetic risk is often associated with early onset of PD (i.e. in monogenic familial forms), individuals having very high OXPHOS-specific mitoPRS could be potential target of preventative treatments.

In sum, the present inventors showed that an OXPHOS-mito gene set is suitable for calculating a polygenic risk score (PRS) in a suitable cohort. However, what is more, the present inventors showed for the first time that a PRS based on an OXPHOS-mito gene set is not only a result obtained by statistics which suggests an association of a high PRS with Parkinson's disease, but indeed reflects a (pathological) biology. Indeed, the present inventors verified what statistics may suggest in that they showed that primary skin fibroblasts and corresponding induced pluripotent stem cells (iPSC)-derived neuronal progenitors from Parkinson's disease patients, which were classified based on mitoPRS (high-risk vs low-risk) have a mitochondrial phenotype. In fact, the inventors found remarkable differences in mitochondrial oxygen consumption between the high- and low-PRS groups, whereas other phenotypic readouts of mitochondrial function, such as mitophagy and mtDNA levels, remained unchanged.

Accordingly, the present invention provides a method for stratifying whether or not a subject may be at a risk of suffering from Parkinson's disease (PD), comprising determining in a sample obtained from said subject its OXPHOS-mito polygenic risk score, wherein a high OXPHOS-mito polygenic risk score is indicative that said subject may be at a risk of developing PD.

Alternatively, the present invention provides a method for stratifying whether or not a subject may be at a risk of suffering from Parkinson's disease (PD), comprising determining in a sample obtained from said subject its OXPHOS-mito polygenic risk score, wherein a low OXPHOS-mito polygenic risk score is indicative that said subject may not be at a risk of developing PD.

The present invention also provides a method for stratifying whether or not a subject suffering from Parkinson's disease (PD) may be at a risk of having mitochondrial dysfunction, comprising determining in a sample obtained from said subject its OXPHOS-mito polygenic risk score (PRS), wherein a high OXPHOS-mito PRS is indicative that said subject may be at a risk of developing mitochondrial dysfunction and/or wherein a low OXPHOS-mito PRS is indicative that said subject may not be at a risk of developing mitochondrial dysfunction. Mitochondrial dysfunction may be characterized by increased mitochondrial oxygen consumption or decreased mitochondrial oxygen consumption. Herein, increased mitochondrial oxygen consumption can also be referred to as mitochondrial hyperactivity.

The present invention also provides a method for stratifying whether or not a subject may be at risk suffering from Parkinson's disease (PD) with an early age of onset, comprising determining in a sample obtained from said subject its OXPHOS-mito polygenic risk score (PRS), wherein a high OXPHOS-mito PRS is indicative that said subject may be at a risk of developing Parkinson's disease (PD) with an early age of onset and/or wherein a low OXPHOS-mito PRS is indicative that said subject may not be at a risk of developing Parkinson's disease (PD) with an early age of onset.

Further, the present invention provides a compound for use in a method for preventing and/or treating Parkinson's disease in a subject, wherein said subject has a high OXPHOS-mito polygenic risk score. Preferably, said compound modulates mitochondrial oxygen consumption. Modulation of mitochondrial oxygen consumption may be an increase of mitochondrial oxygen consumption or a decrease of mitochondrial oxygen consumption.

A compound for the prevention and/or treating Parkinson's Disease according to the present disclosure may modulate mitochondrial oxygen consumption. As such, modulation of mitochondrial oxygen consumption may be an increase of mitochondrial oxygen consumption or a decrease of mitochondrial oxygen consumption. A compound for the prevention and/or treating Parkinson's disease according to the present disclosure may be a drug that targets one or more electron transport chain proteins, preferably one or more electron transport chain complex subunits, wherein the one or more electron transport chain proteins, preferably the one or more electron chain complex subunits, are preferably selected from the group consisting of complexes I, II, III, and IV, and ATP synthase. Preferably, such a compound is selected from the group consisting of Nobiletin, NAHS, Compound A and rT1. Alternatively, such a compound is (Coenzyme) Q10, also referred to as CoQ (see Formula (I), C59H9004). Suitable compounds are described in Xu et al 2022, Acta Pharmaceutica Sinica B 2022; 12(6):2778e2789; Amarsanaa K et al., Exp Neurobiol 2021; 30:73e86; Kumar M et al. Mitochondrion 2020; 50:158e69; Jiang X et al., Mol Cell 2016; 63:229e39; Kam A et al. J Biol Chem 2019; 294:4000e11. In particular, the polymethoxylated flavonoid 5,6,7,8,3′,4′-hexamethoxyflavone, also referred to as Nobiletin, has been reported to act on Complex I and has been shown to restore activity of complex I in a rat model (Amarsanaa K et al., Exp Neurobiol 2021; 30:73e86). Nobiletin has the molecular formula C21H2208 shown in Formula (II). NaHS has been proposed for increasing mitochondiral complex I, II and IV mediated oxygen consumption rate (Kumar M et al. Mitochondrion 2020; 50:158e69). Small molecule Compound A has been designed to target complex II, preventing dopaminergic neurons' death in a rat PD model (Jiang X et al., Mol Cell 2016; 63:229e39). Compound A is also referred to as 1-(3,4-dimethoxybenzyl)-5-(2-(methylsulfonyl)-6-(trifluoromethyl)pyrimidin-4-yl) pyridin-2 (1H) and shown in Formula (III). The 27-residue cysteine-rich peptide rT1 has been shown to prompt ATP synthase and cell survival by targeting ETC (rT1 has the sequence CIPRGGICLVALSGCCNSPGCIFGICA shown in SEQ ID NO: 1); Kam A et al. J Biol Chem 2019; 294:4000e11). Preferably, such a compound shows preclinical and/or clinical evidence of targeting one ore more mitochondrial OXPHOS defects. Thus, an especially envisioned compound for the prevention and/or treating Parkinson's disease according to the present disclosure is a drug targeting electron transport chain (ETC) complex subunit mediating oxidative phosphorylation and/or energy conversion in mitochondria, preferably with the potential to improve OXPHOS activity.

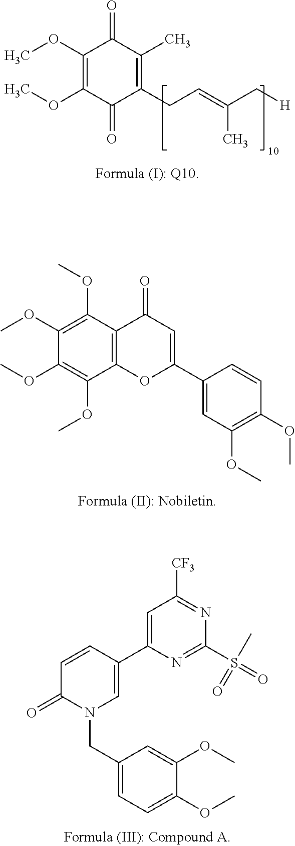

Also, the present invention provides for a use of a cell being characterized by a high OXPHOS-mito polygenic risk score for screening compounds for the prevention or treatment of Parkinson's disease. Preferably, said compound modulates mitochondrial oxygen consumption. Modulation of mitochondrial oxygen consumption may be an increase of mitochondrial oxygen consumption or a decrease of mitochondrial oxygen consumption.

Said cell may be a fibroblast, an induced pluripotent stem cell (iPSC) or induced pluripotent stem cell (iPSC)-derived neuronal progenitor cell (smNPCs). Herein, the terms “stem cell (iPSC)-derived neuronal progenitor cell” (or “smNPC”) and “neuroepithelial stem cell” (or “NESC”) are used interchangeably.

Moreover, the present invention provides a method for screening compounds for the prevention or treatment of Parkinson's disease, comprising bringing a cell being characterized by a high OXPHOS-mito polygenic risk score into contact with said compound and determining whether said compound modulates mitochondrial oxygen consumption of said cell. Modulation of mitochondrial oxygen consumption may be an increase of mitochondrial oxygen consumption or a decrease of mitochondrial oxygen consumption.

Said cell may be a fibroblast, an induced pluripotent stem cell (iPSC) or induced pluripotent stem cell (iPSC)-derived neuronal progenitor cell (smNPC).

The present invention also relates to a composition comprising a cell being characterized by a high OXPHOS-mito polygenic risk score, optionally further comprising a buffer. Said cell may be a fibroblast, an induced pluripotent stem cell (iPSC) or induced pluripotent stem cell (iPSC)-derived neuronal progenitor cell (smNPC). The composition may further comprise a compound that modulates mitochondrial oxygen consumption of said cell, such as a compound that is for the prevention and/or treating Parkinson's Disease according to the present disclosure.

Further, the present invention relates to a system comprising a cell being characterized by a high OXPHOS-mito polygenic risk score, optionally further comprising a buffer. Preferably, said system is adapted for screening compounds which decreases/increases mitochondrial oxygen consumption of said cell. Said cell may be a fibroblast, an induced pluripotent stem cell (iPSC) or induced pluripotent stem cell (iPSC)-derived neuronal progenitor cell (smNPC). The system may further comprise a compound that modulates mitochondrial oxygen consumption of said cell, such as a compound that is for the prevention and/or treating Parkinson's Disease according to the present disclosure.

Furthermore, the present invention relates to a compound obtained or obtainable by the methods of the present invention.

The present invention also relates to a method for stratifying whether or not a subject suffering from PD may be eligible for a treatment with a compound according to any one of claims 6-11 and 26, comprising determining in a sample obtained from said subject its OXPHOS-mito polygenic risk score (PRS), wherein a high OXPHOS-mito PRS is indicative that said subject may be eligible for treatment with said compound and/or wherein a low OXPHOS-mito PRS is indicative that said subject may not be eligible for treatment with said compound.

It is understood that a Parkinson's Disease according to the present disclosure is preferably idiopathic Parkinson's Disease. It is also preferred that a Parkinson's Disease according to the present disclosure is preferably associated with mitochondrial dysfunction and/or aberrant mitochondrial oxygen consumption.

Whole genome PD PRS can be calculated according to methods well established in the art. For example, one can use the PRSice2 software (preferably v.2.3.5 2021 Sep. 20) under default settings (Choi and O'Reilly, 2019). PRSs for the different mitochondrial gene sets can be generated using the PRSet function in PRSice2. PRSs for each individual can be calculated by summing the weighted effects of the risk alleles associated with PD-based on the largest summary statistics of PD GWAS to date (Nalls et al., 2019).

The OXPHOS-mito polygenic risk score is preferably calculated on the basis of the gene set described in Paliwal et al. (DOI: 10.1016/j.neurobiolaging.2021.08.005) in Table 2.

The present disclosure envisages that the OXPHOS-mito polygenic risk score can be calculate using a gene set comprising genes that are associated with the mitochondrial oxidative phosphorylation pathway. Such genes are not necessarily limited to mitochondrial genes, but can also comprise non-mitochondrial genes that are nonetheless involved in the oxidative phosphorylation pathway.

The OXPHOS-mito polygenic risk score can be calculated using genes that are listed as Oxidative Phoshorylation (OXPHOS-mito) genes in Table 9. Preferably, the OXPHOS-mito polygenic risk score is obtained using a gene set comprising at least about 50, at least about 60, at least about 70, at least about 80, at least about 90, at least about 100, at least about 110, at least about 115, at least about 120, such as at least about 121, at least about 122, at least about 123, or about 124 of the OXPHOS-mito genes listed in Table 9. Preferably, the OXPHOS-mito polygenic risk score is obtained using a gene set comprising the OXPHOS-mito genes listed in Table 9. Preferably, the OXPHOS-mito polygenic risk score is obtained using a gene set consisting of OXPHOS-mito genes listed in Table 9.

The OXPHOS-mito polygenic risk score can be calculated using human genes that have the Gene Ontology Database Pathways IDs of GO:0006119 (Oxidative phosphorylation), GO:0090324 (Negative regulation of oxidative phosphorylation), and GO:1903862 (Positive regulation of oxidative phosphorylation), preferably according to Gene Ontology Database release 2022 Jul. 1. Preferably, the OXPHOS-mito polygenic risk score is obtained using a gene set comprising at least about 50, at least about 60, at least about 70, at least about 80, at least about 90, at least about 100, at least about 110, at least about 115, at least about 120, such as at least about 121, at least about 122, at least about 123, or about 124 human genes having the Gene Ontology Database Pathways IDs of GO:0006119 (Oxidative phosphorylation), GO:0090324 (Negative regulation of oxidative phosphorylation), and GO:1903862 (Positive regulation of oxidative phosphorylation). Preferably, the OXPHOS-mito polygenic risk score is obtained using a gene set comprising all human genes having the Gene Ontology Database Pathways IDs of GO:0006119 (Oxidative phosphorylation), GO:0090324 (Negative regulation of oxidative phosphorylation), and GO:1903862 (Positive regulation of oxidative phosphorylation). Preferably, the OXPHOS-mito polygenic risk score is obtained using a gene set consisting of human genes having the Gene Ontology Database Pathways IDs of GO:0006119 (Oxidative phosphorylation), GO:0090324 (Negative regulation of oxidative phosphorylation), and GO:1903862 (Positive regulation of oxidative phosphorylation).

The OXPHOS-mito polygenic risk score can be calculated using genes that are listed as Oxidative Phoshorylation (OXPHOS-mito) genes selected from the group consisting of ABCD1, ACTN3, AFG1L, ATP5F1A, ATP5F1B, ATP5F1C, ATP5F1D, ATP5F1E, ATP5MC1, ATP5MC2, ATP5MC3, ATP5ME, ATP5MF, ATP5MG, ATP5PB, ATP5PD, ATP5PF, ATP5PO, ATP7A, BID, CCNB1, CDK1, CHCHD10, COA6, COQ9, COX10, COX15, COX411, COX412, COX5A, COX5B, COX6A1, COX6A2, COX6B1, COX6C, COX7A1, COX7A2, COX7A2L, COX7B, COX7C, COX8A, CYC1, CYCS, DLD, DMAC2L, DNAJC15, DNAJC30, FXN, MECP2, MLXIPL, MSH2, MYOG, NDUFA1, NDUFA10, NDUFA11, NDUFA12, NDUFA13, NDUFA2, NDUFA3, NDUFA4, NDUFA5, NDUFA6, NDUFA7, NDUFA8, NDUFA9, NDUFAB1, NDUFAF1, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFC2-KCTD14, NDUFS1, NDUFS2, NDUFS3, NDUFS4, NDUFS5, NDUFS6, NDUFS7, NDUFS8, NDUFV1, NDUFV2, NDUFV3, NIPSNAP2, PARK7, PDE12, PGK1, PGK2, PINK1, PPIF, RHOA, SDHA, SDHAF2, SDHC, SDHD, SHMT2, SLC25A23, SLC25A33, SNCA, STOML2, SURF1, TAZ, TEFM, UQCC2, UQCC3, UQCR10, UQCR11, UQCRB, UQCRC1, UQCRC2, UQCRFS1, UQCRH, UQCRHL, UQCRQ, and VCP. Preferably, the OXPHOS-mito polygenic risk score is obtained using a gene set comprising at least about 50, at least about 60, at least about 70, at least about 80, at least about 90, at least about 100, at least about 110, at least about 115, at least about 120, such as at least about 121, at least about 122, at least about 123, or about 124 of the OXPHOS-mito genes selected from the aforementioned group. Preferably, the OXPHOS-mito polygenic risk score is obtained using a gene set comprising all OXPHOS-mito genes of the aforementioned group. Preferably, the OXPHOS-mito polygenic risk score is obtained using a gene set consisting of OXPHOS-mito genes of the aforementioned group.

Generally, the OXPHOS-mito polygenic risk score can be calculated (obtained) using GWAS data from any suitable dataset comprising PD patients, preferably using a summary statistics of a PD GWAS.

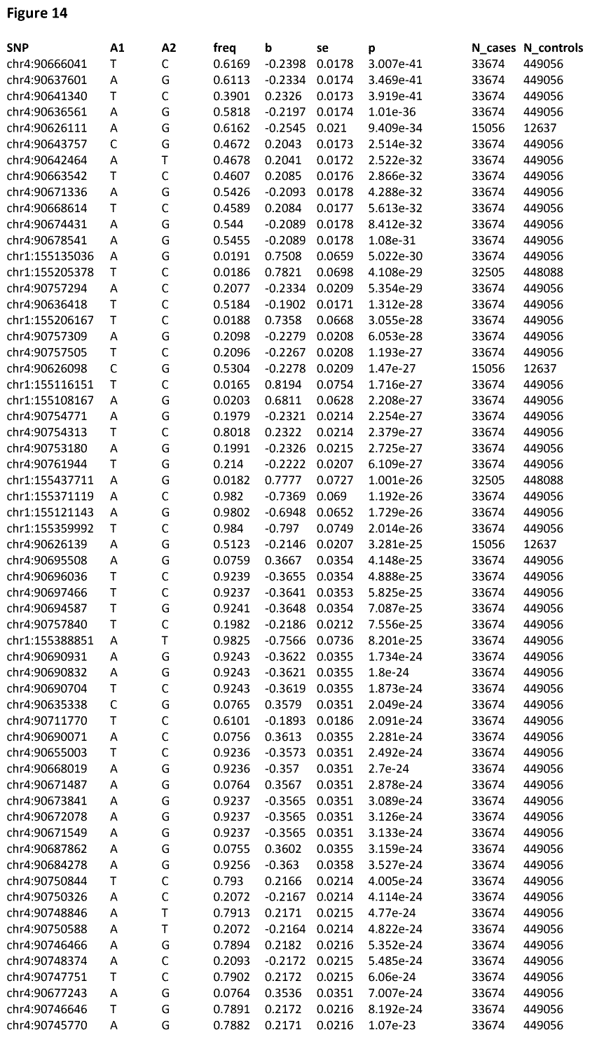

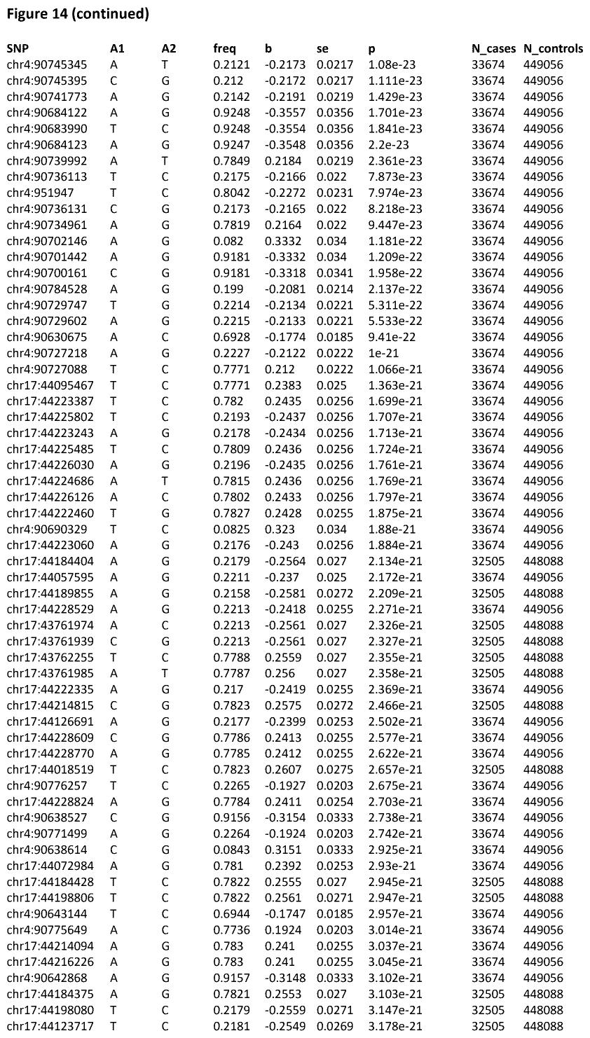

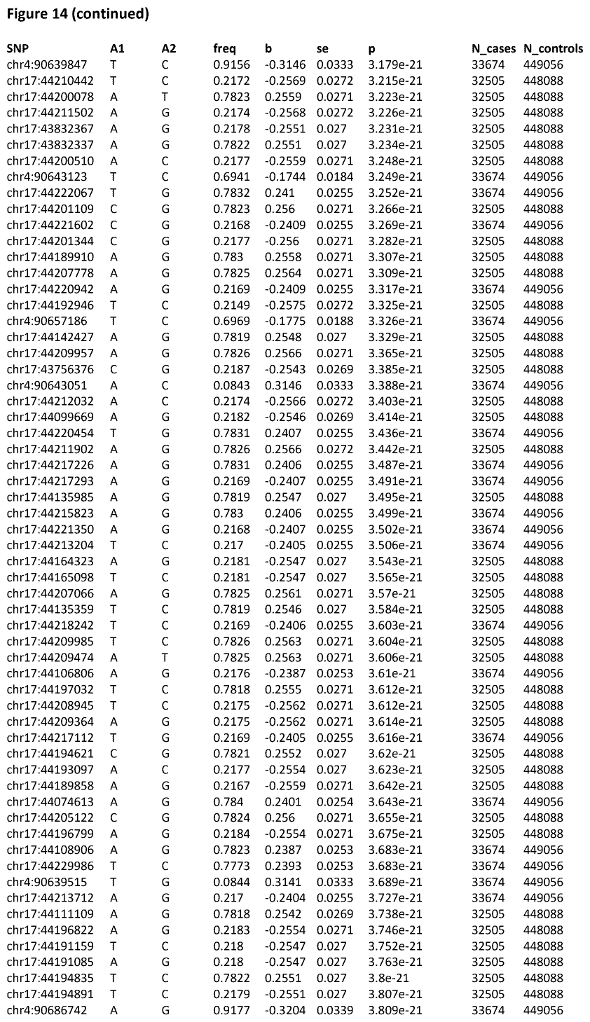

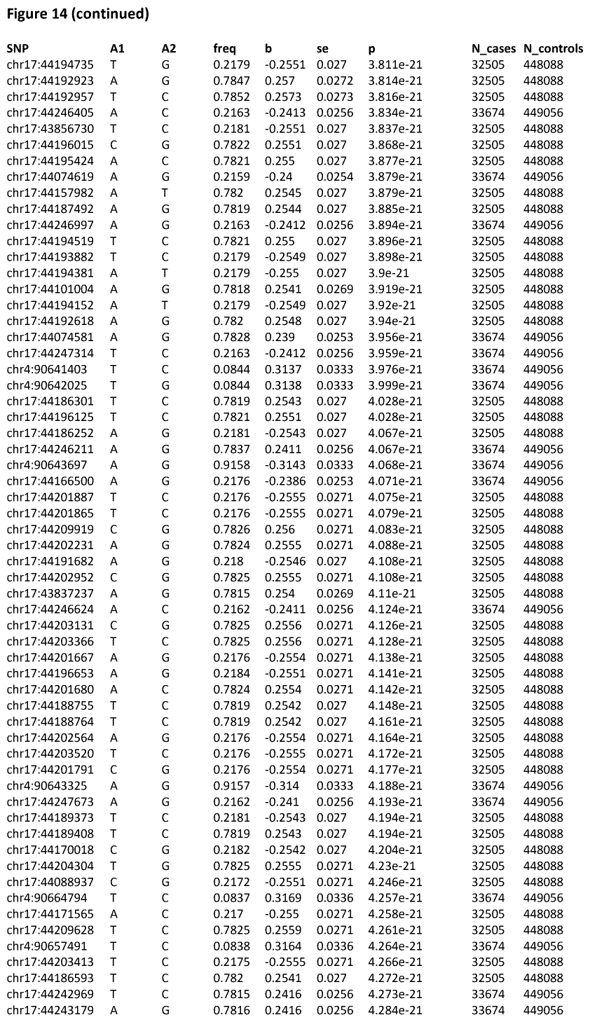

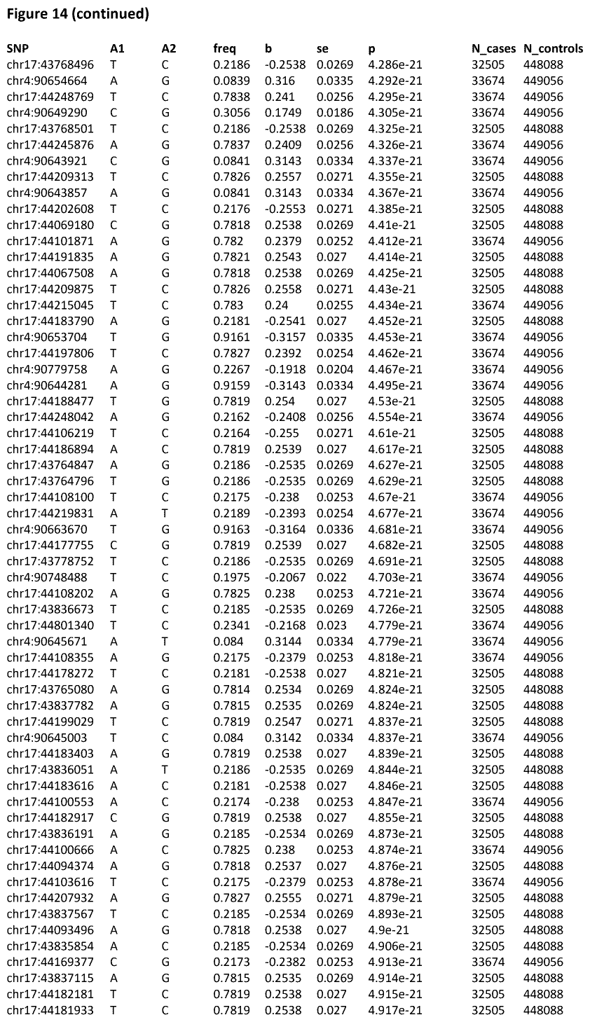

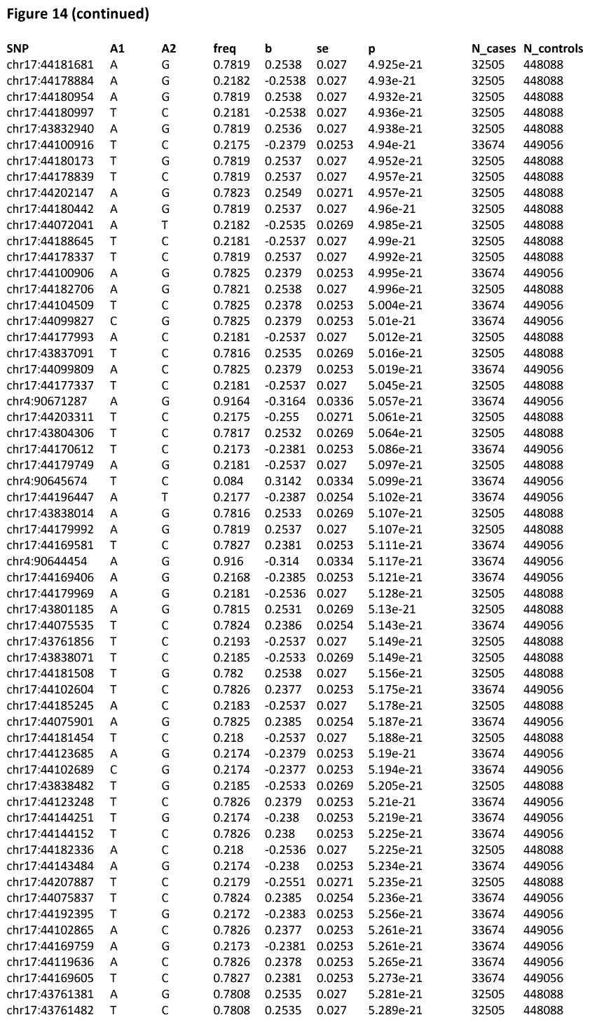

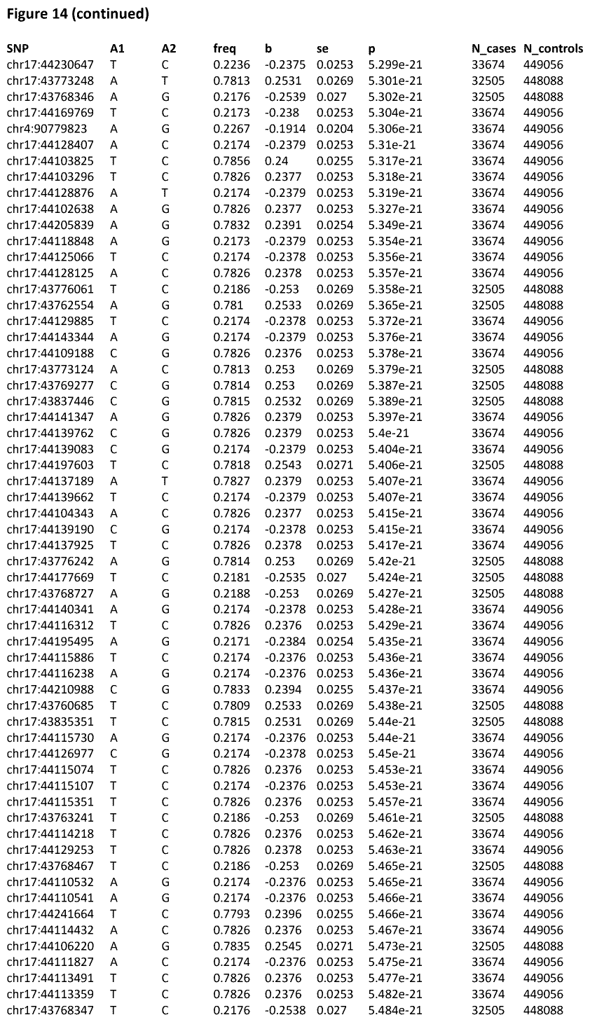

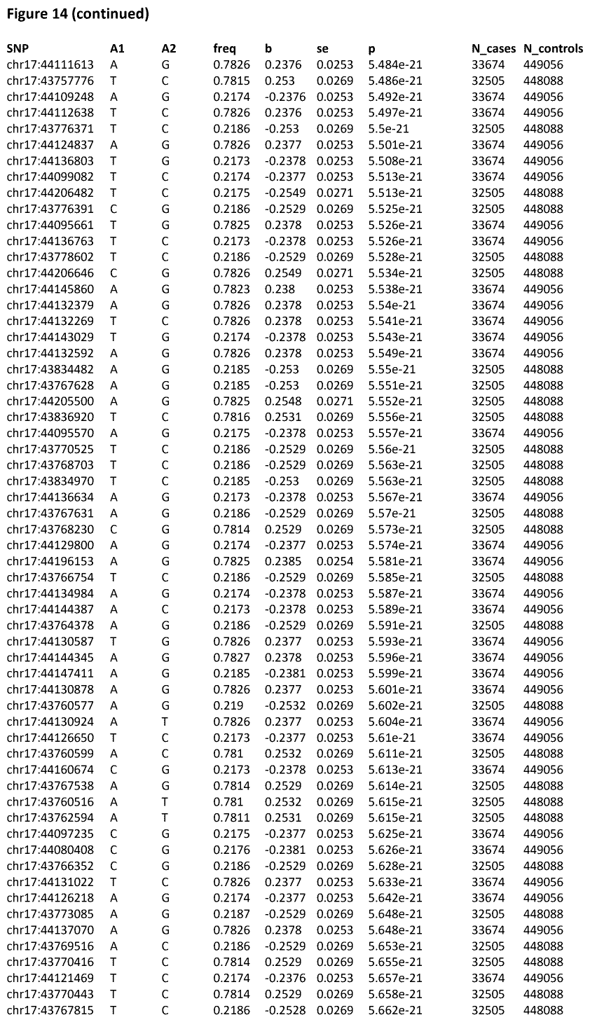

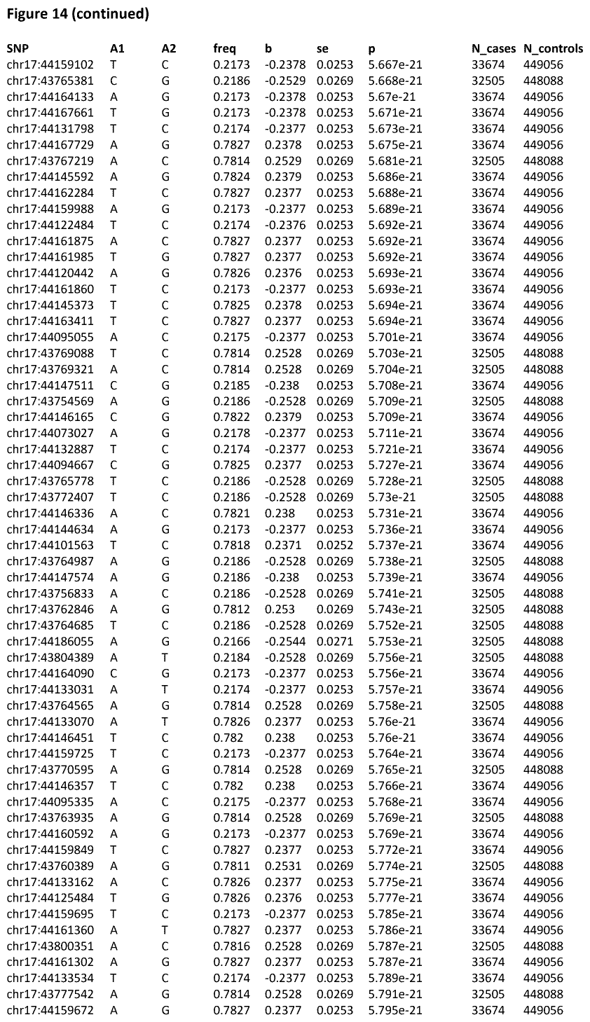

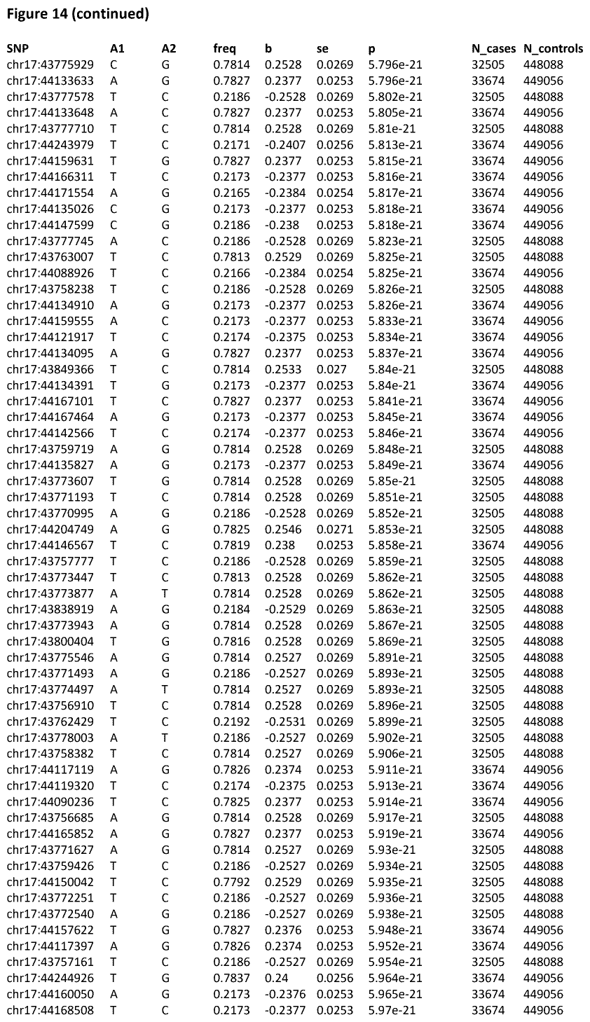

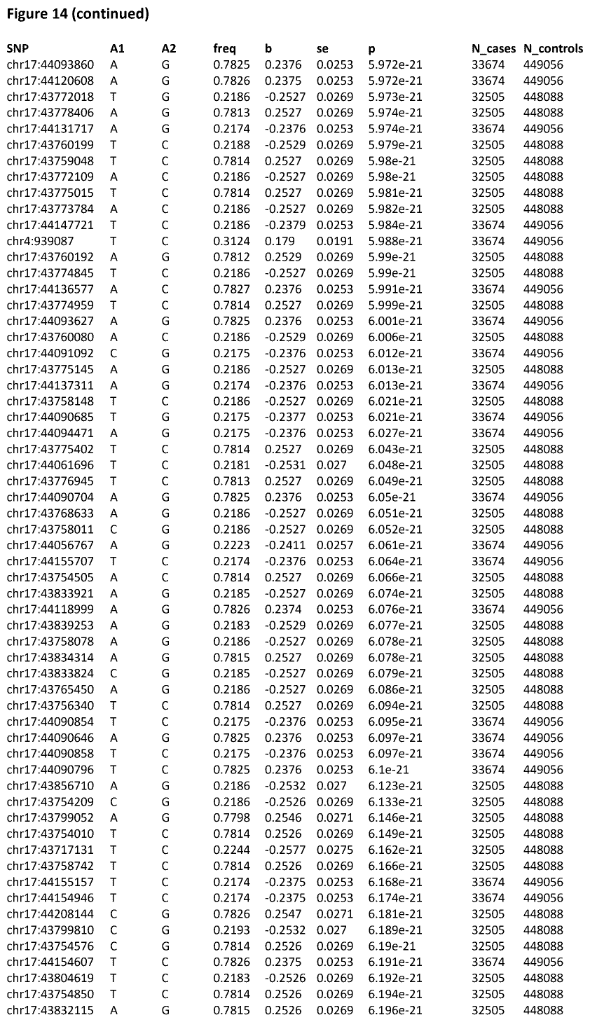

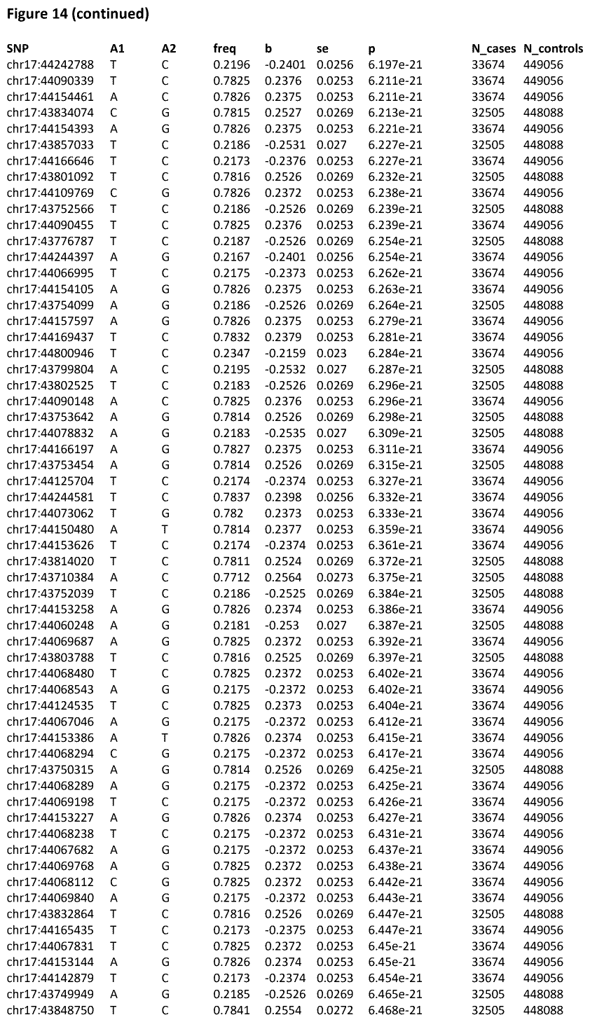

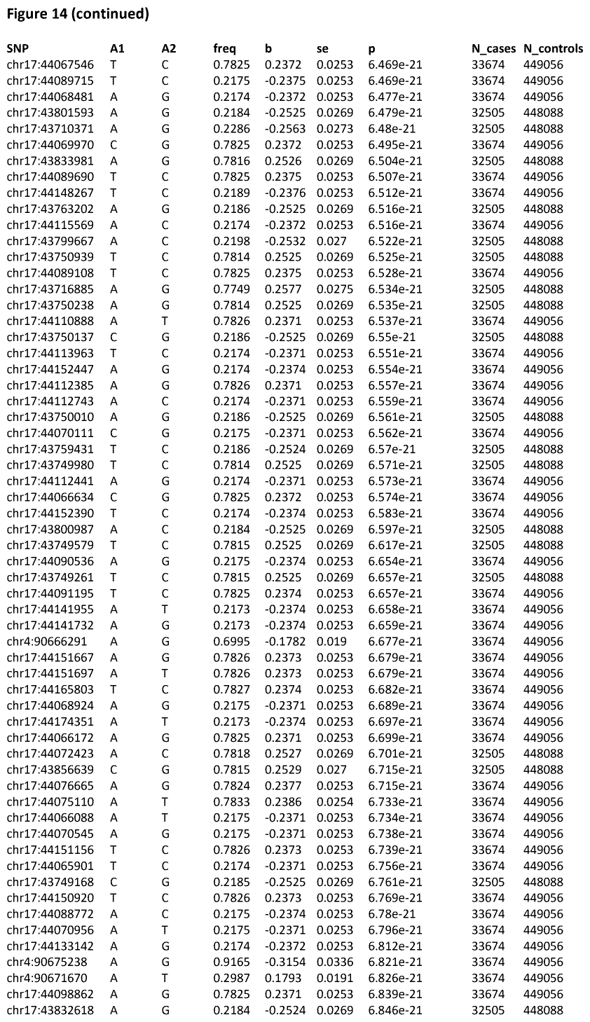

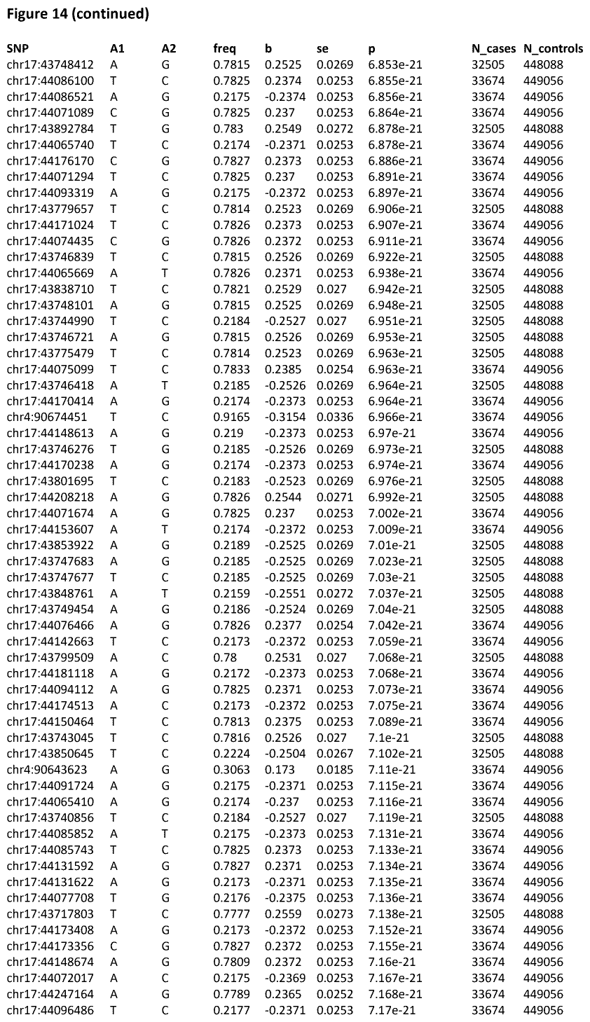

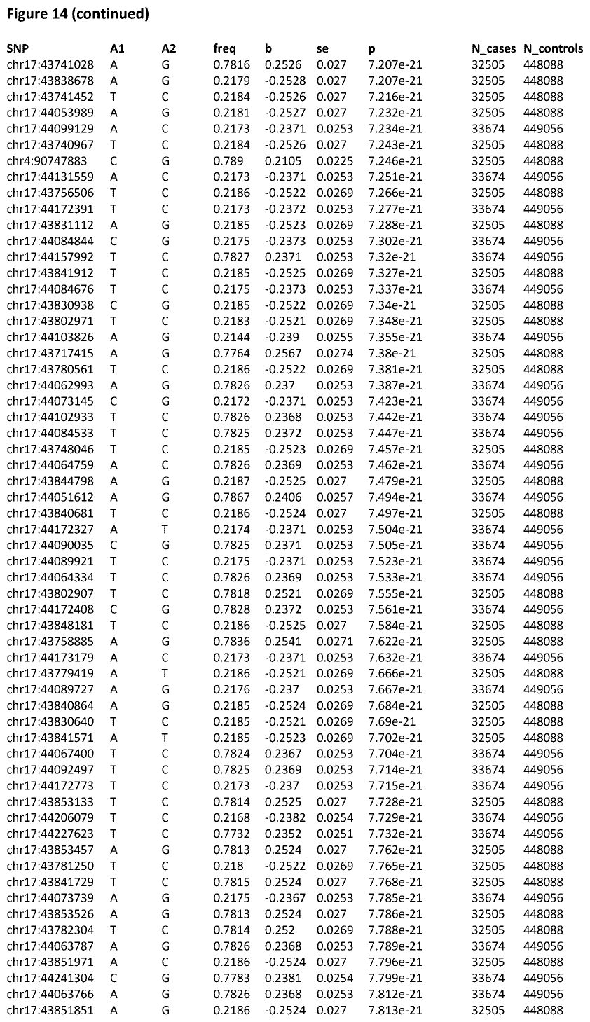

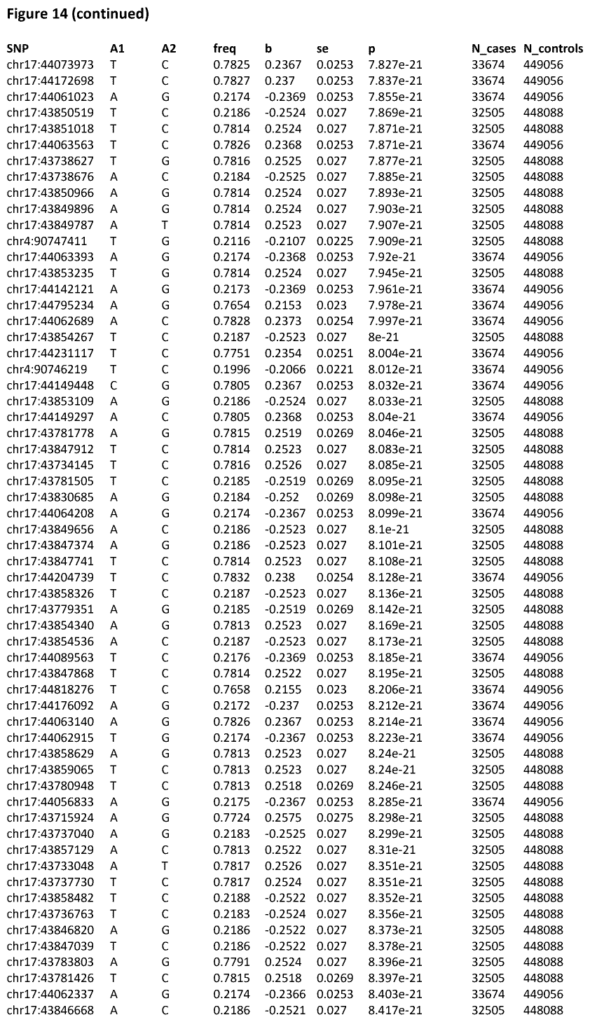

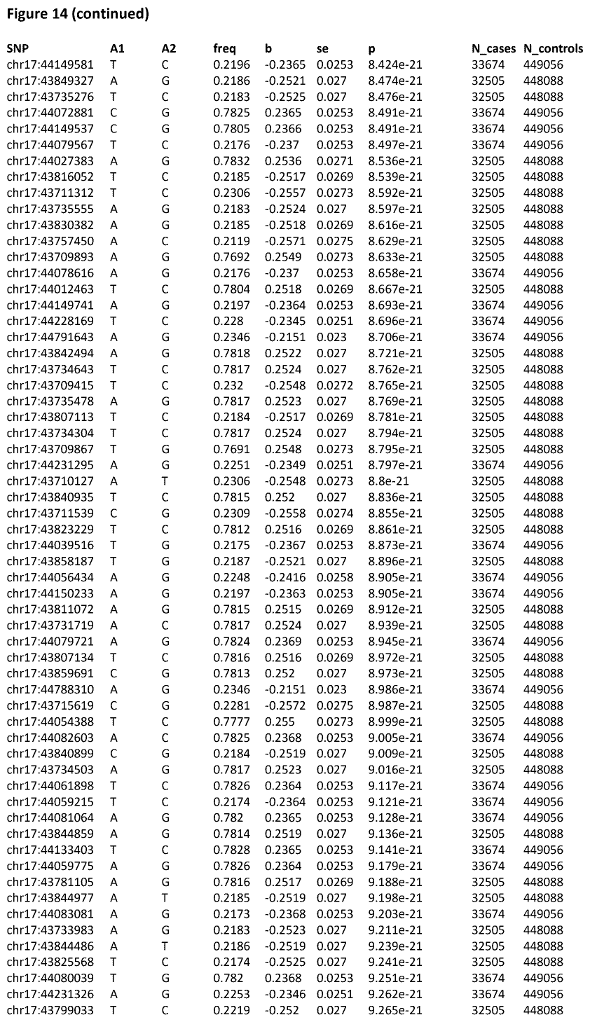

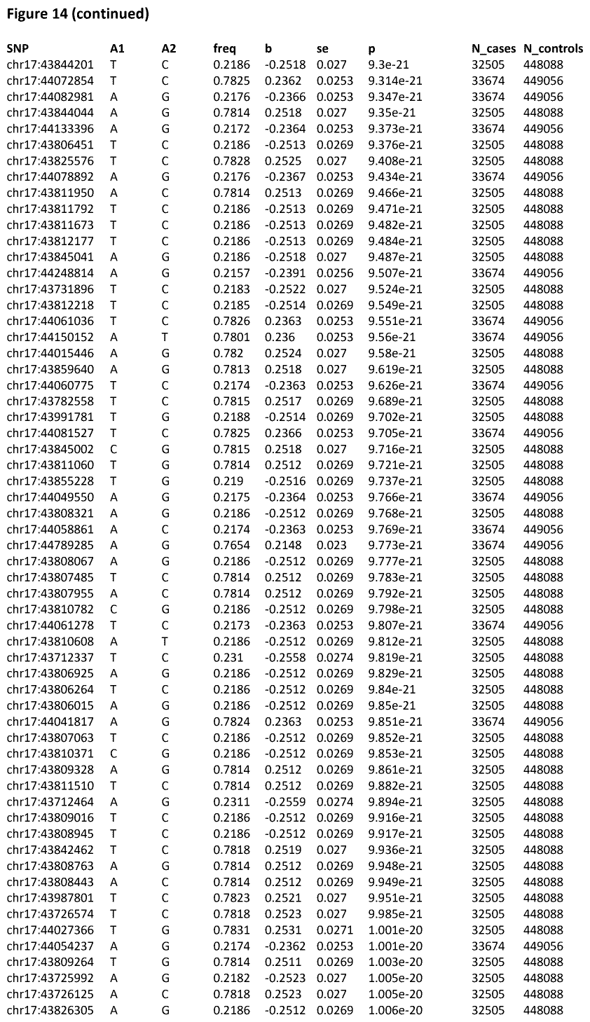

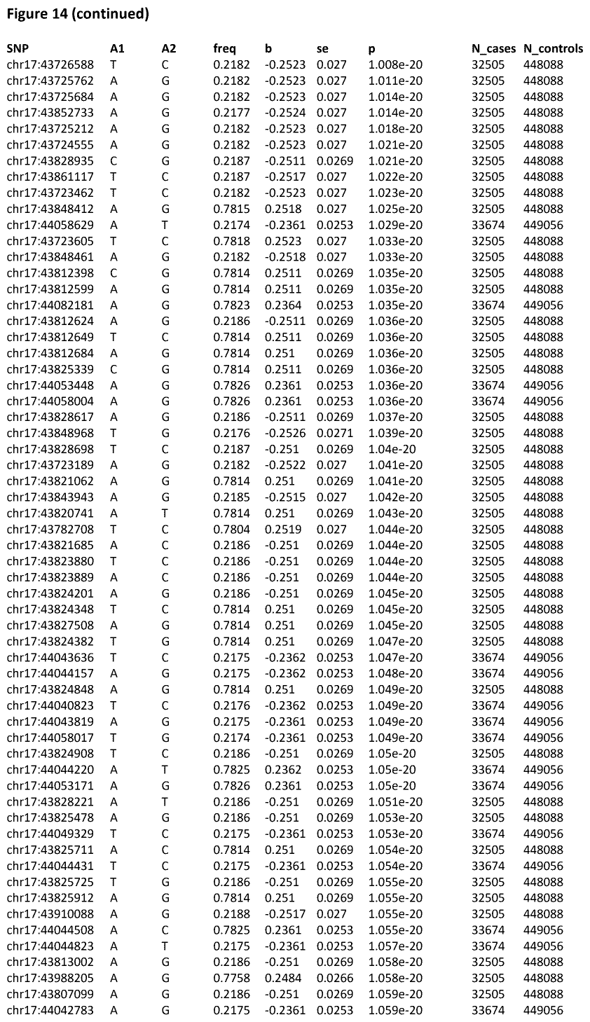

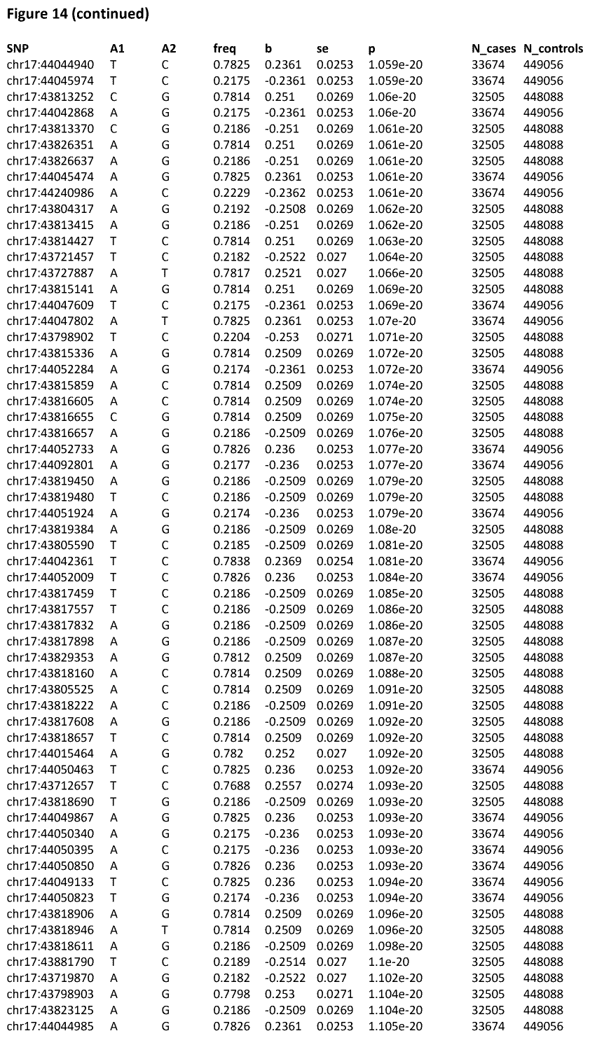

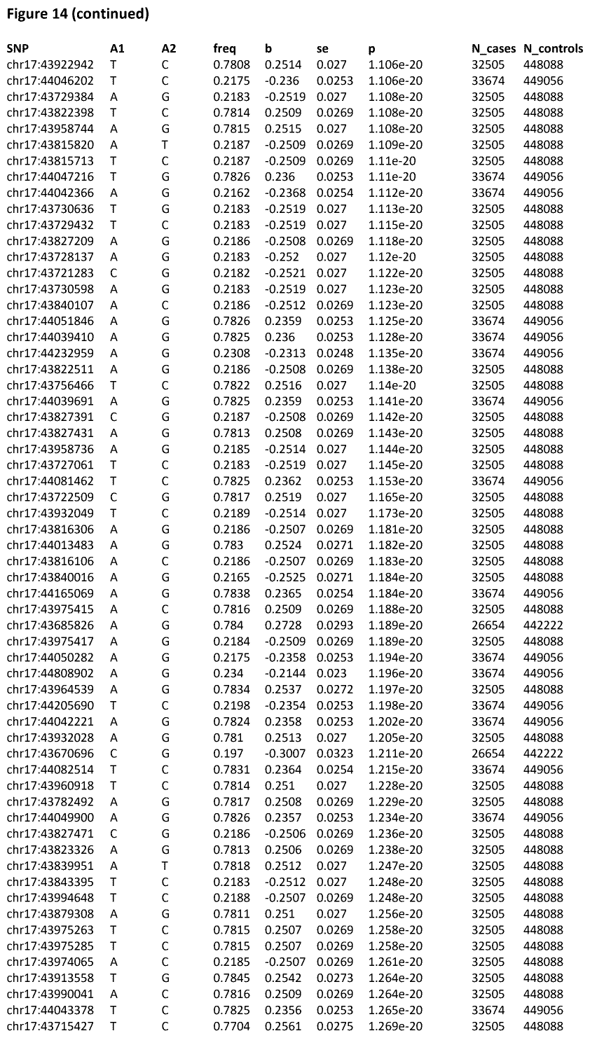

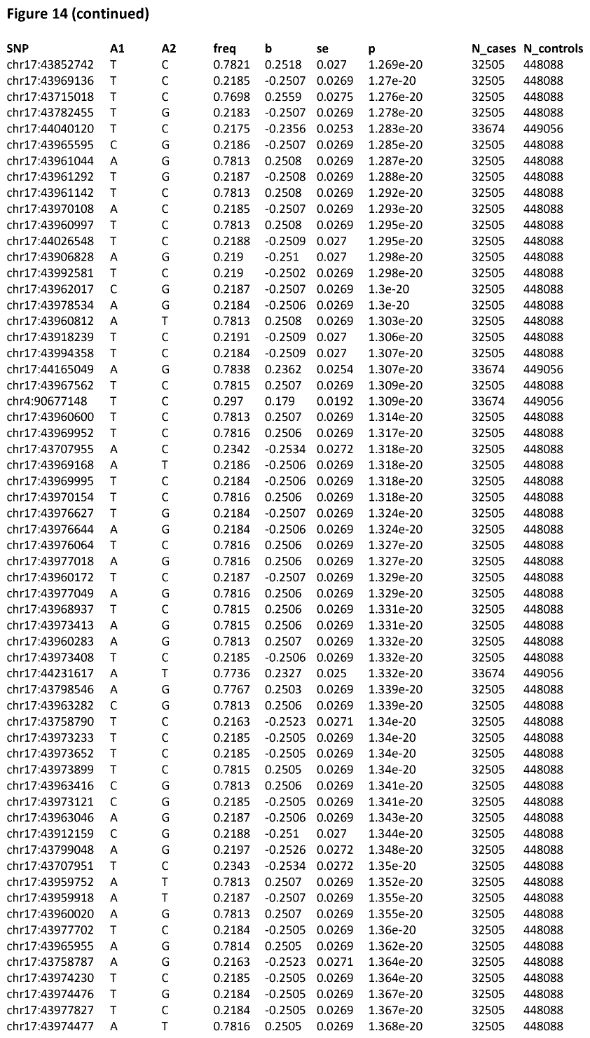

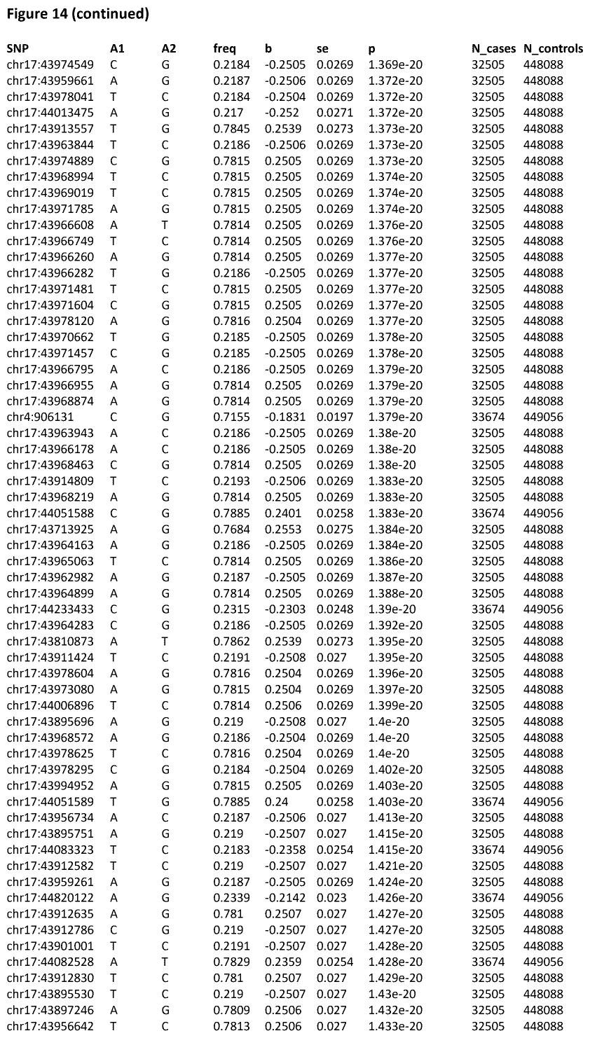

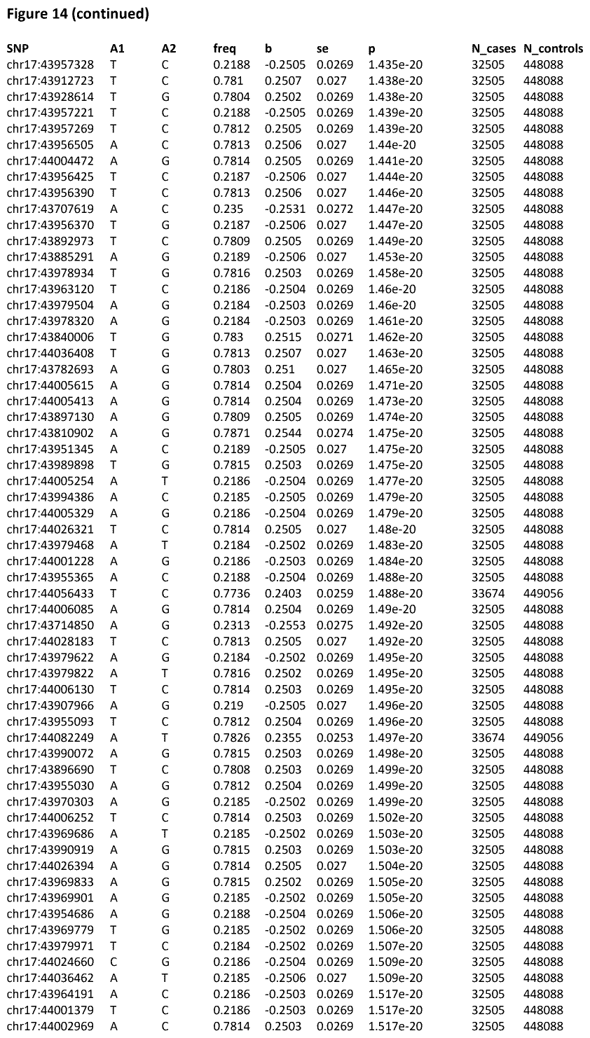

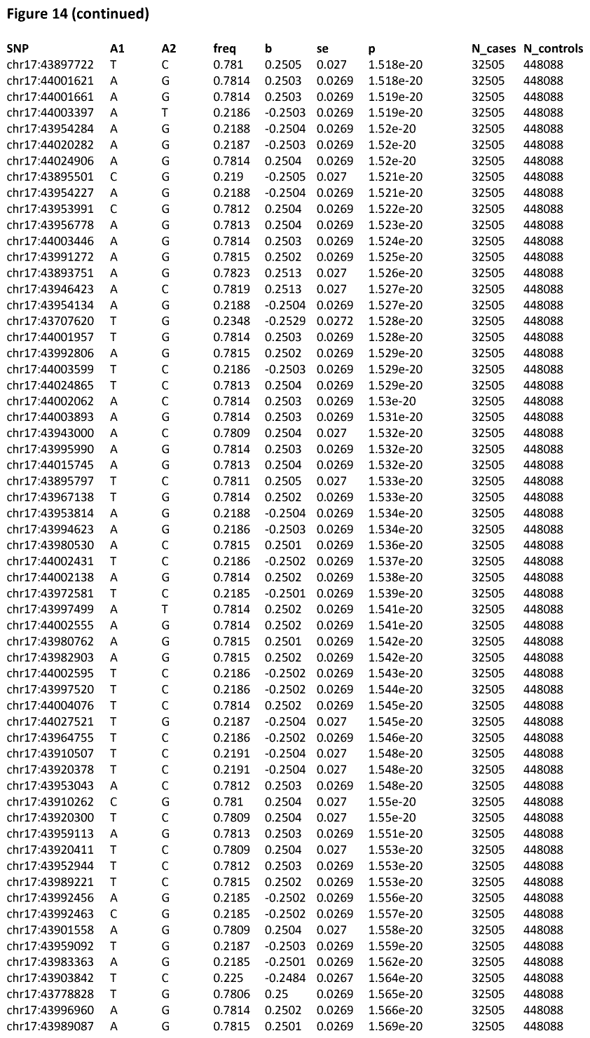

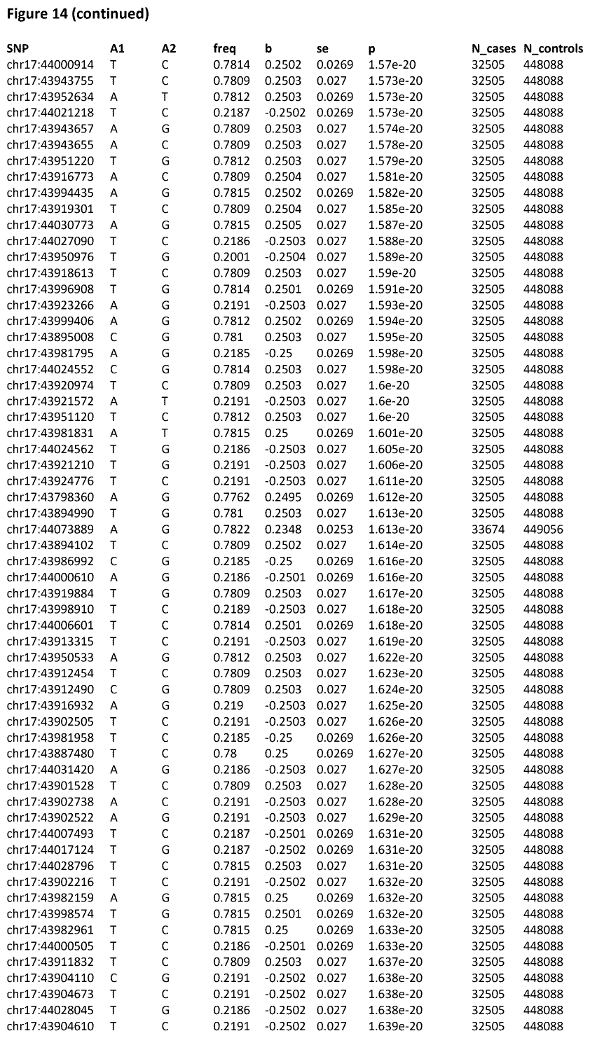

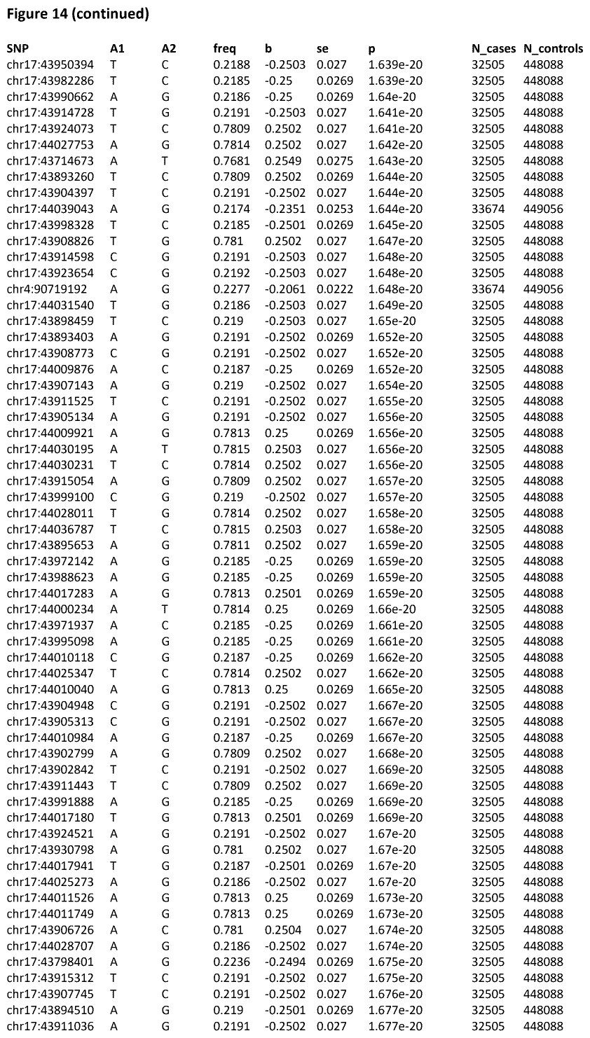

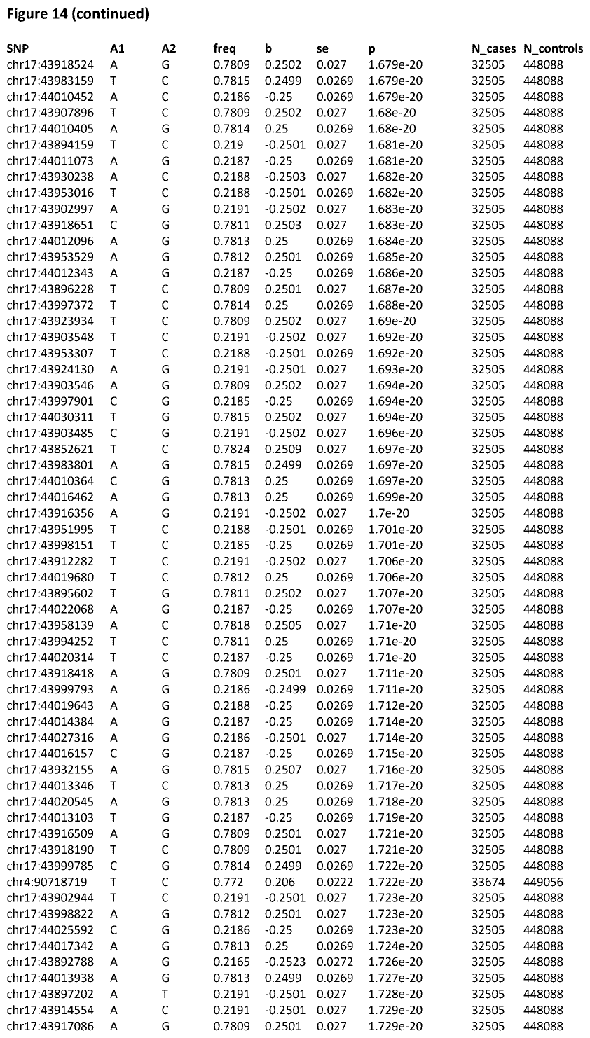

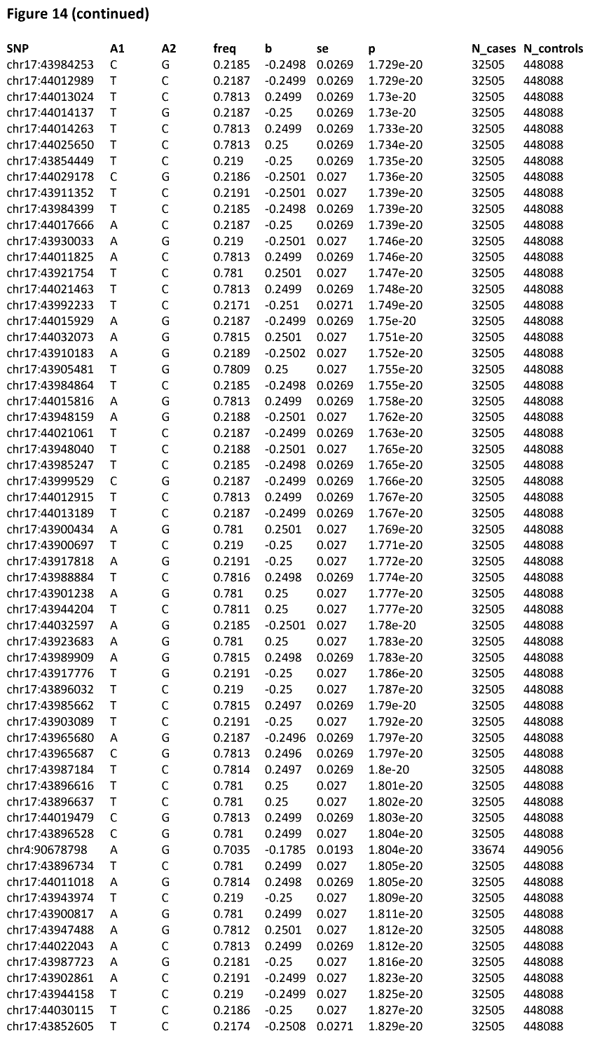

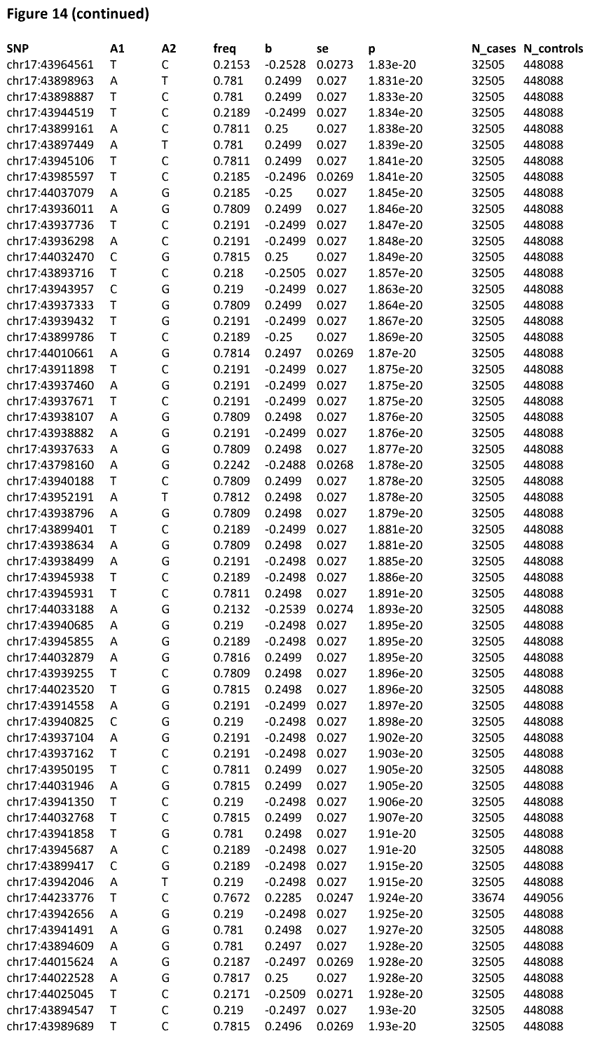

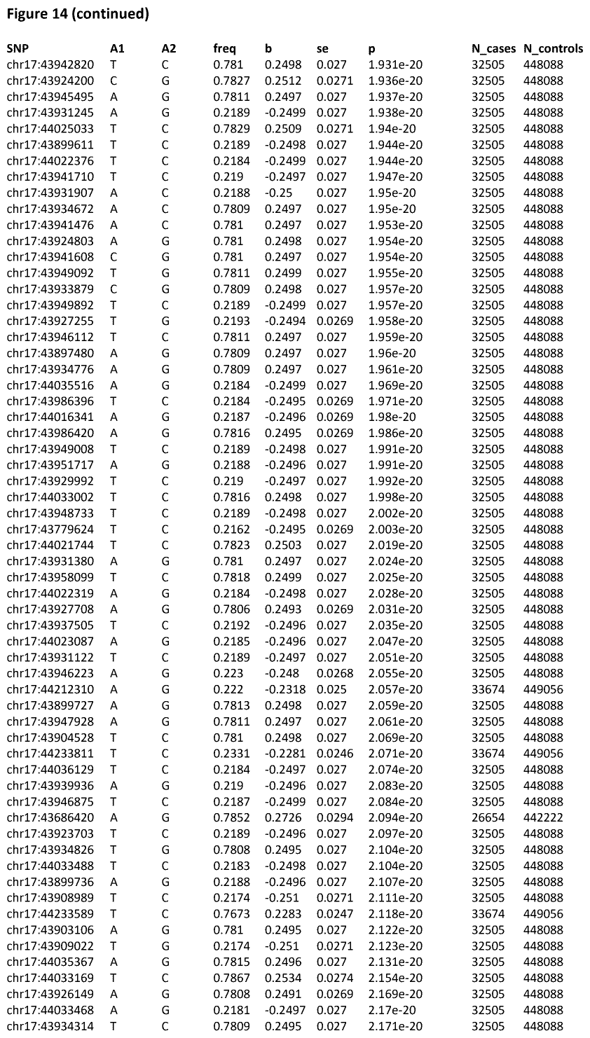

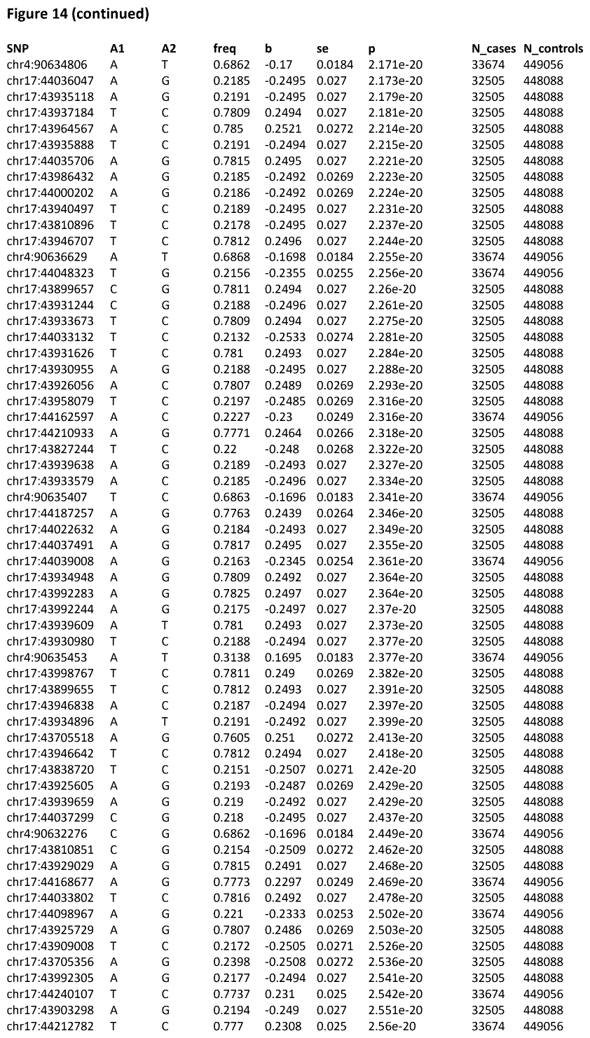

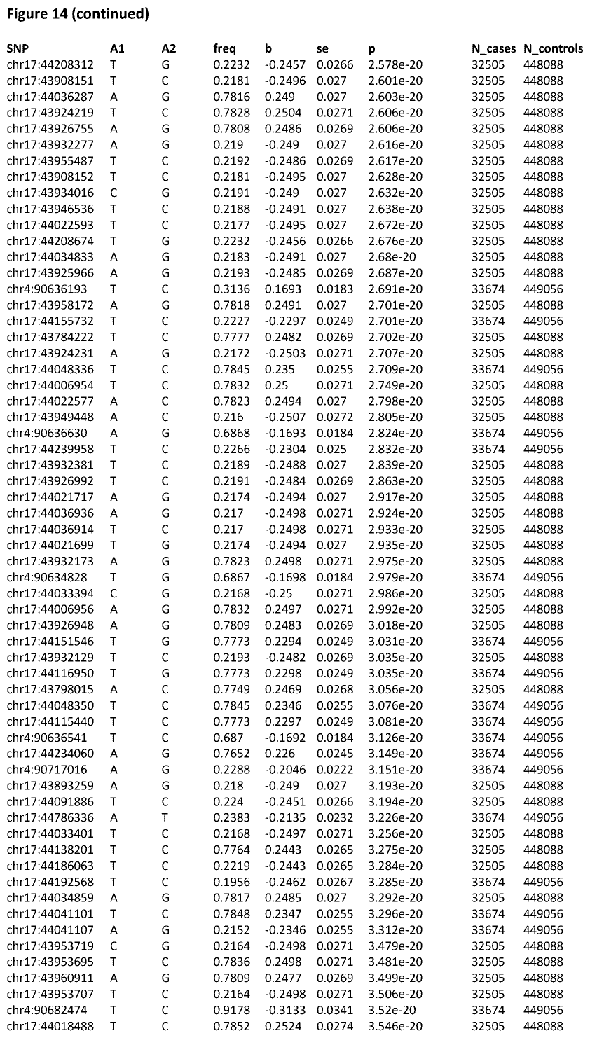

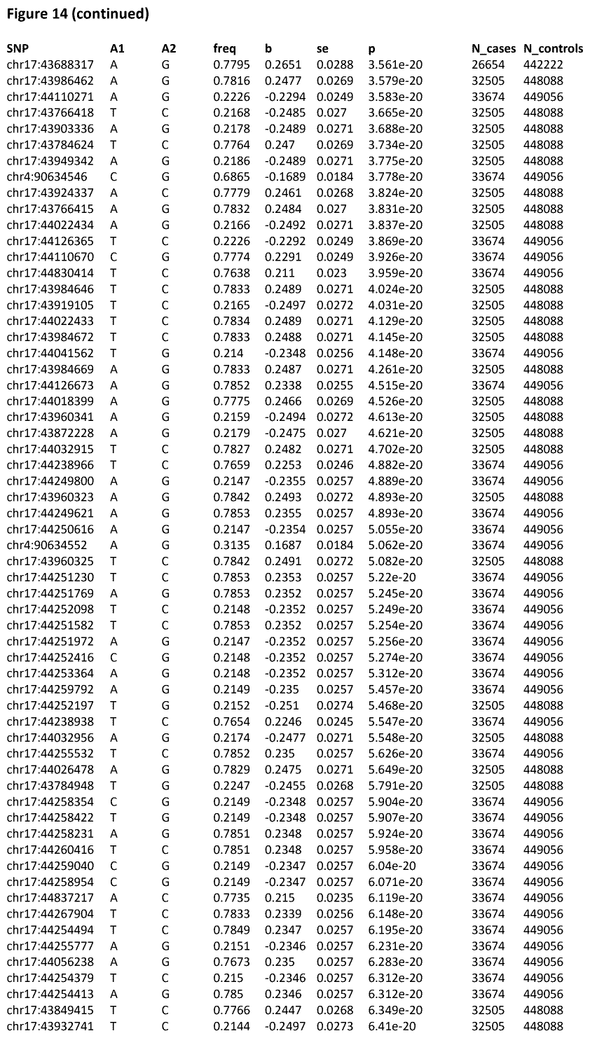

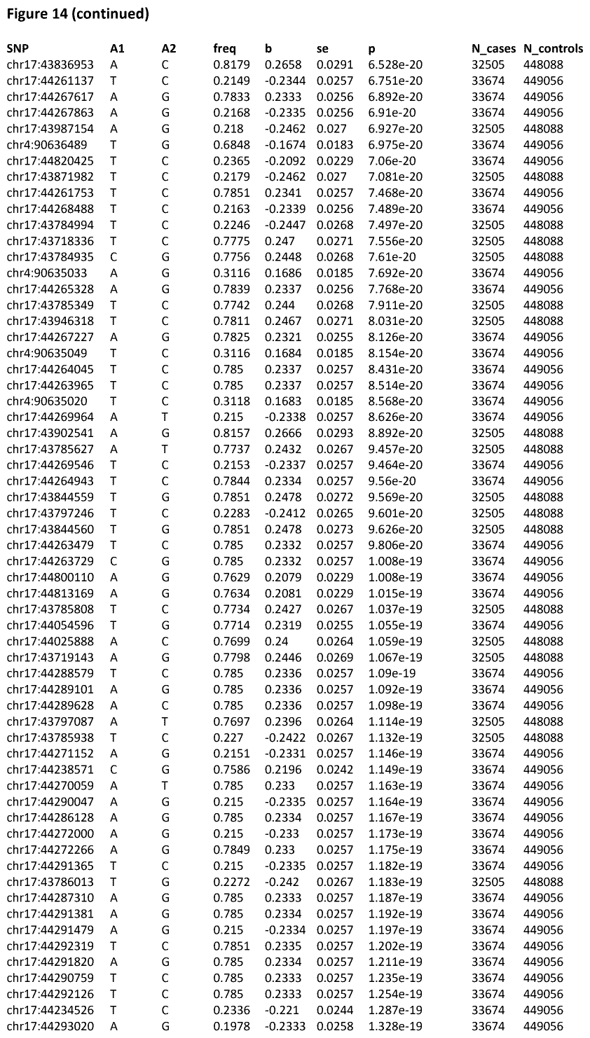

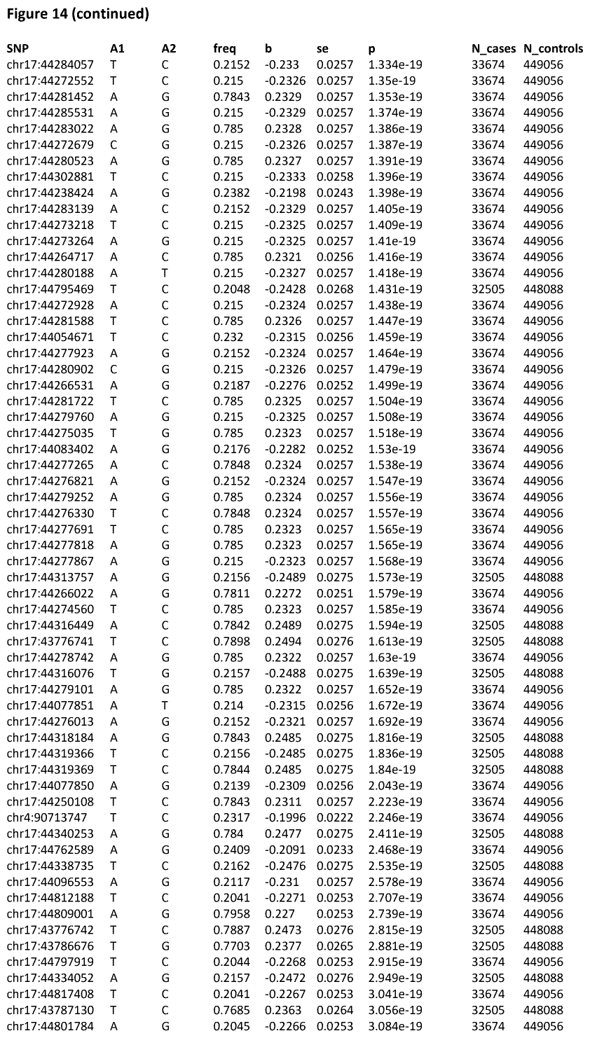

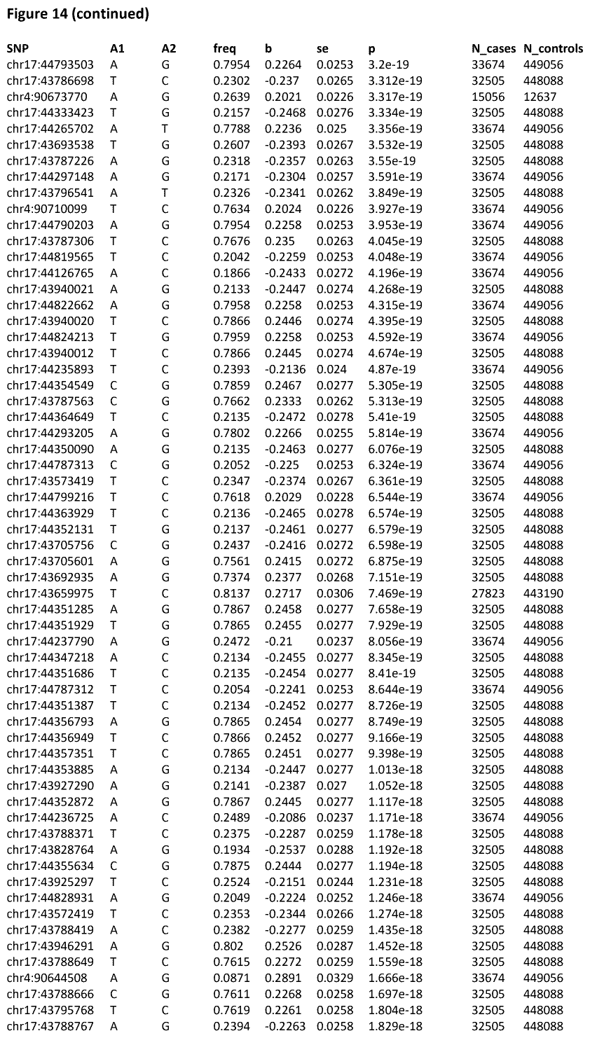

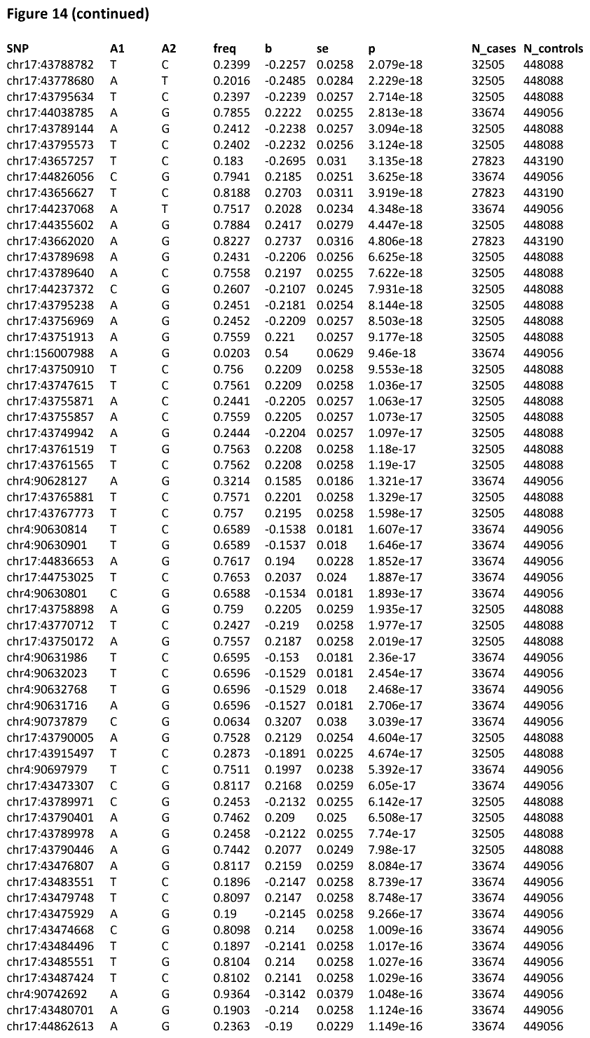

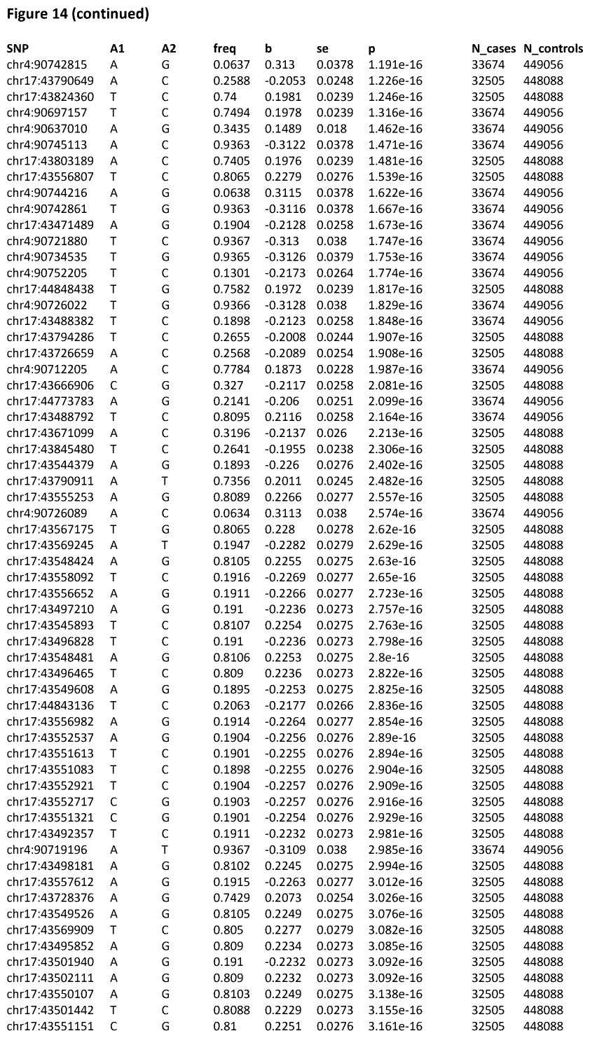

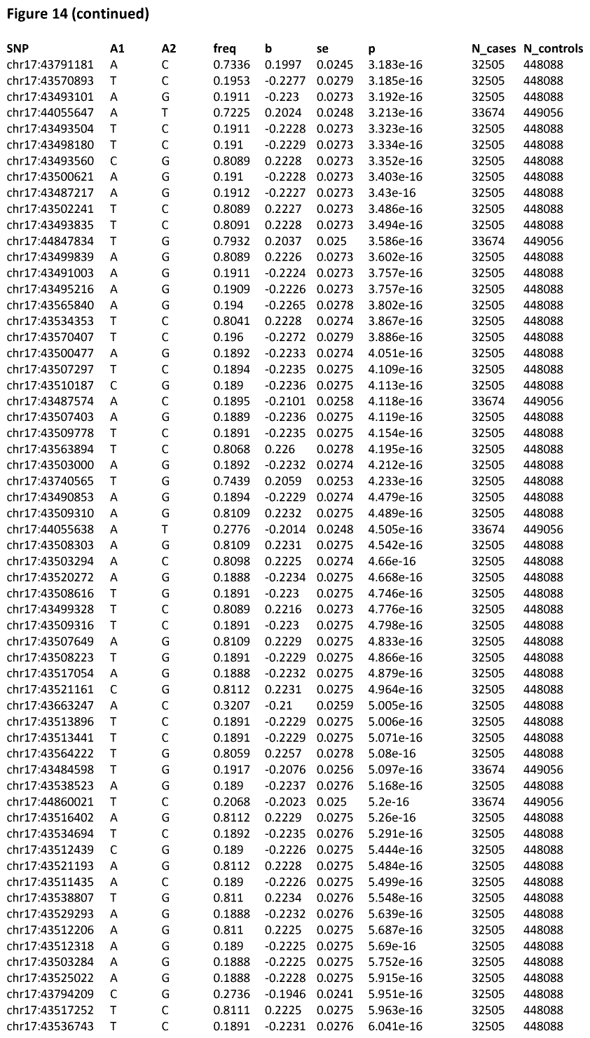

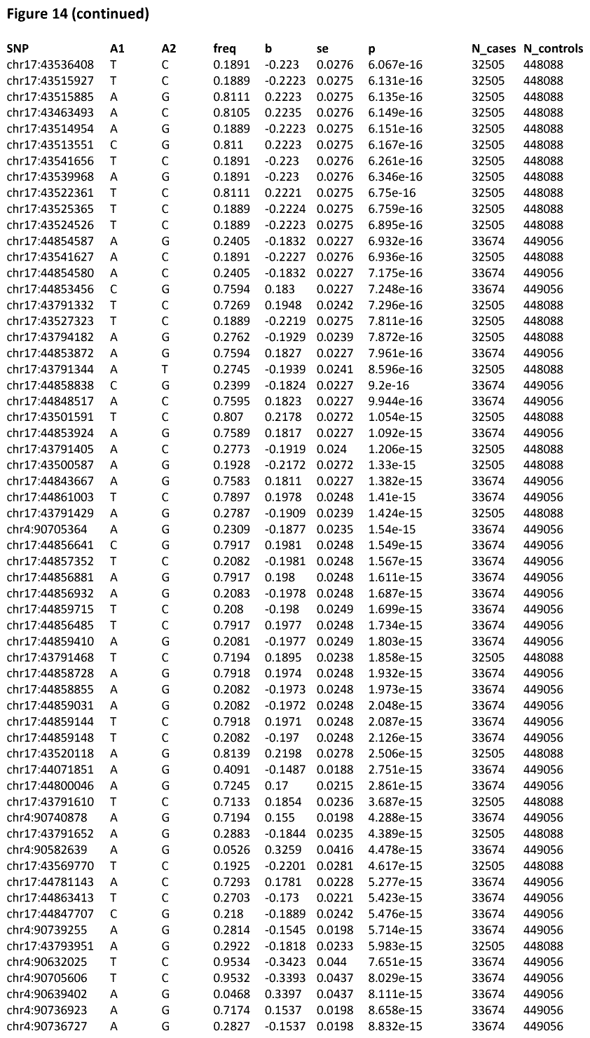

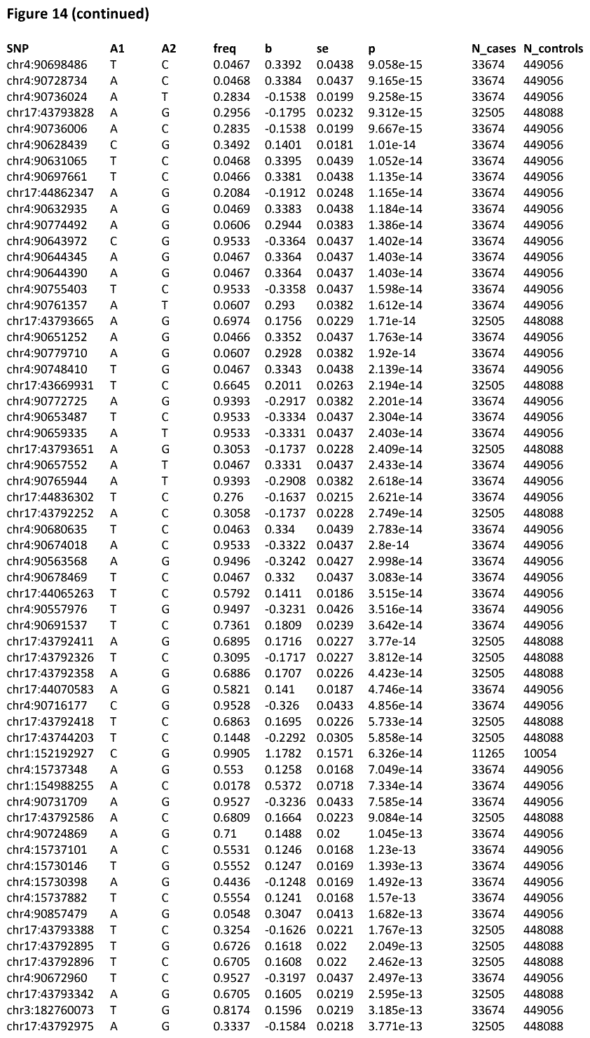

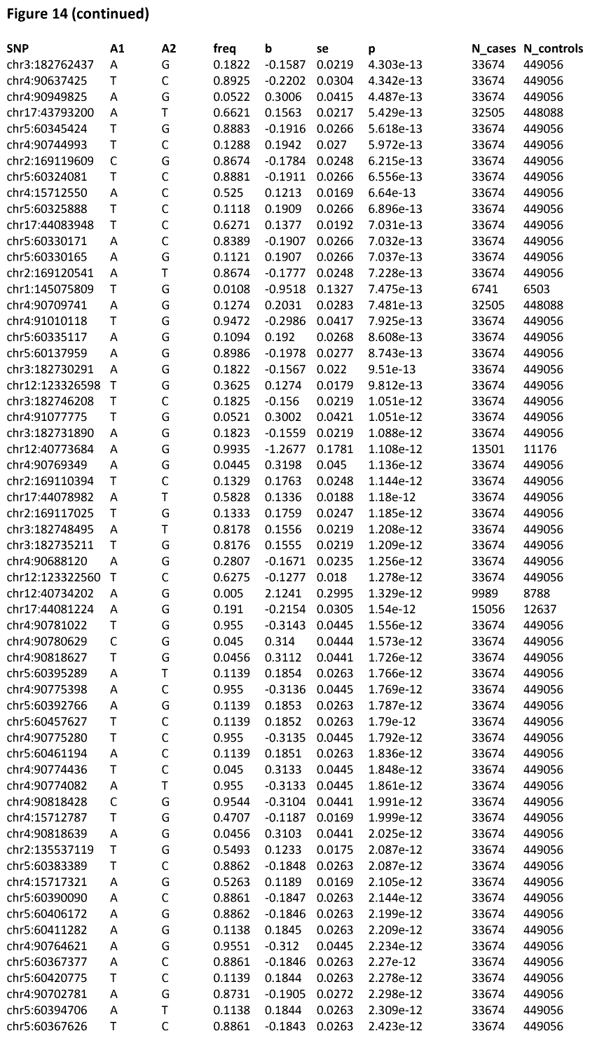

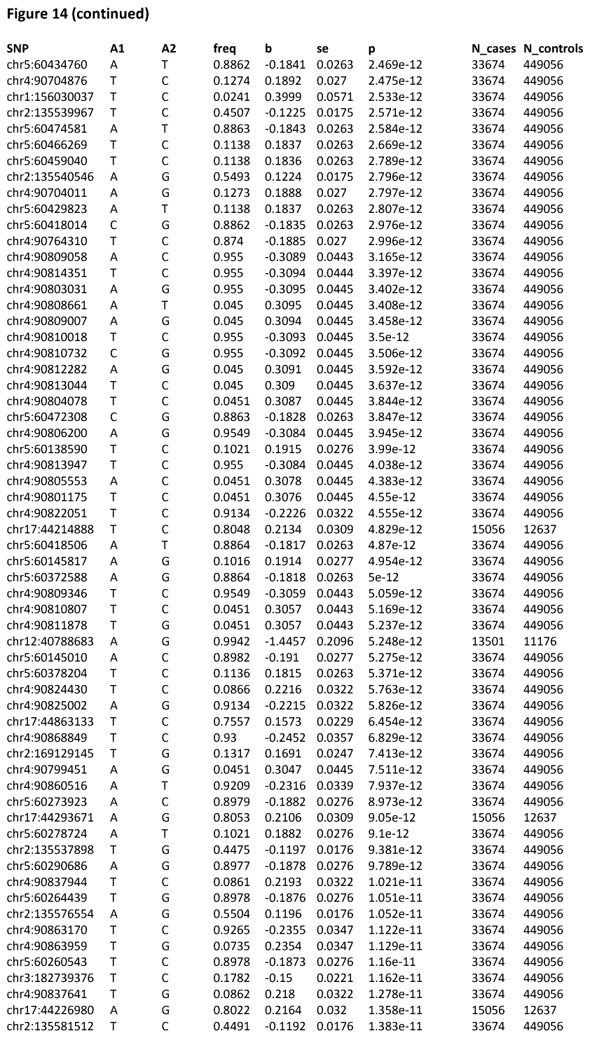

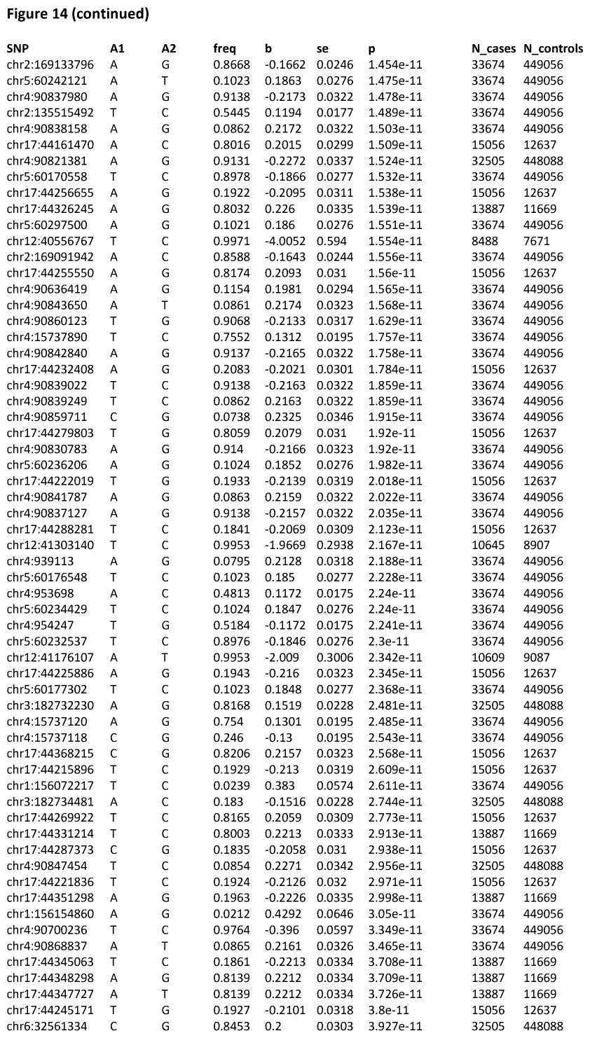

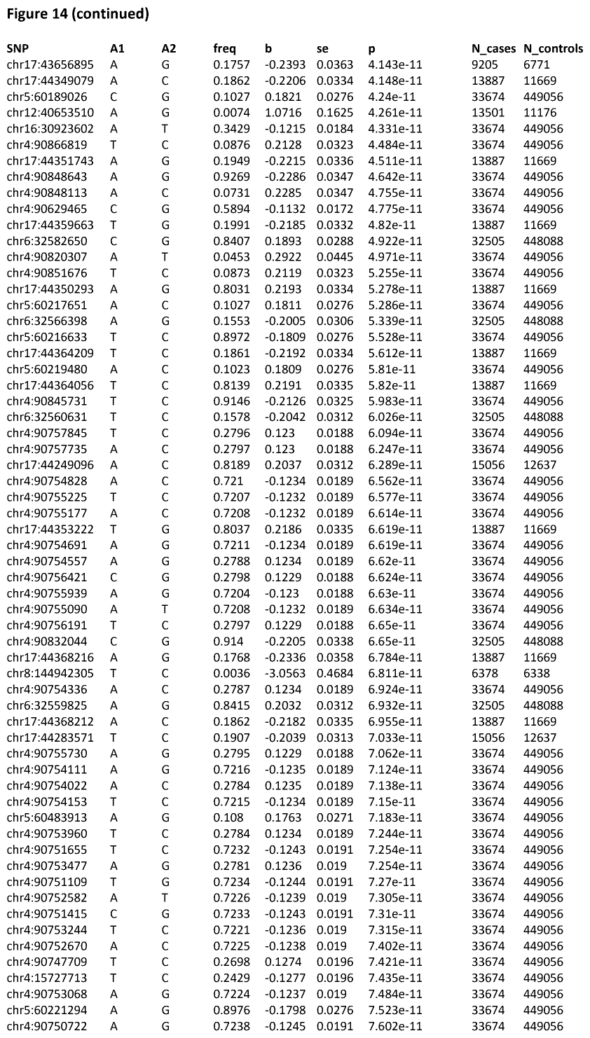

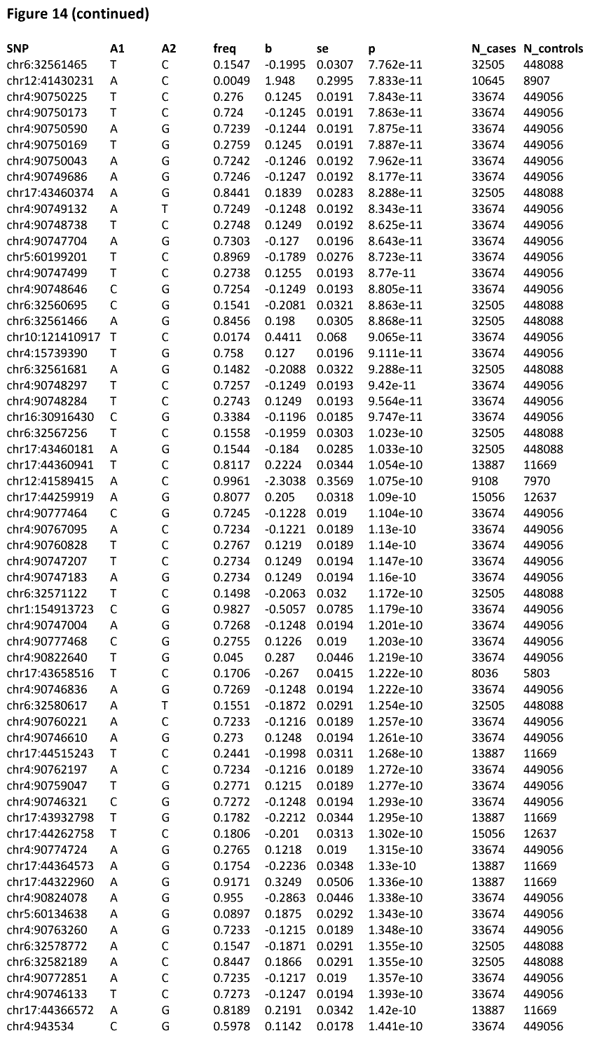

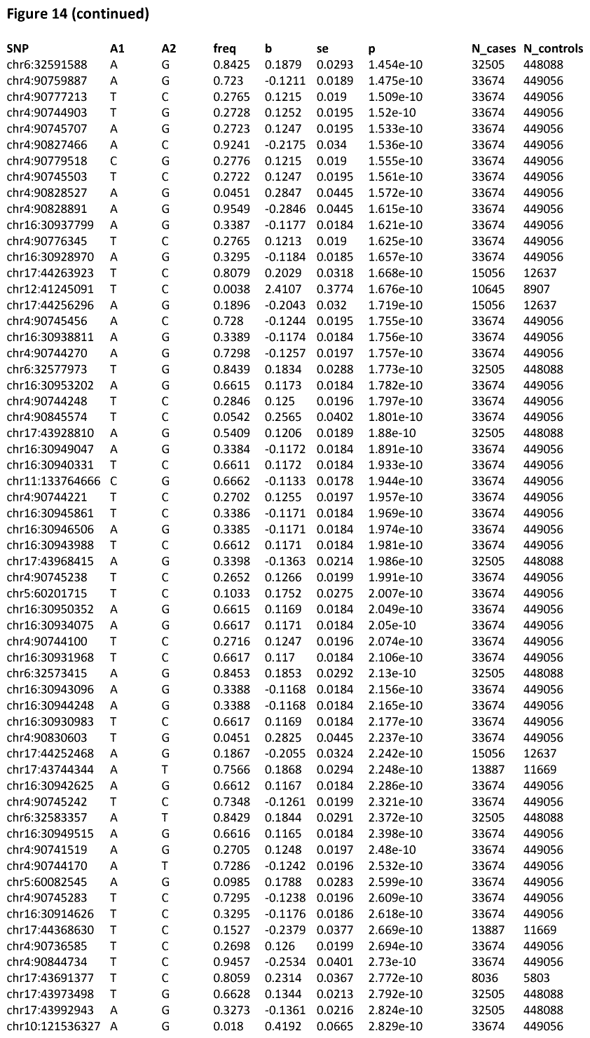

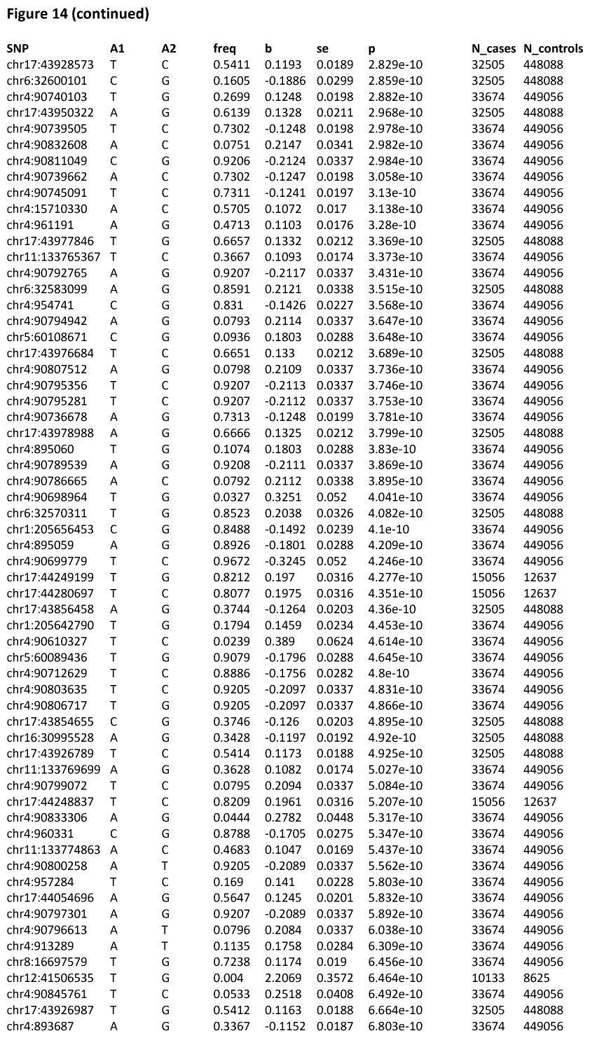

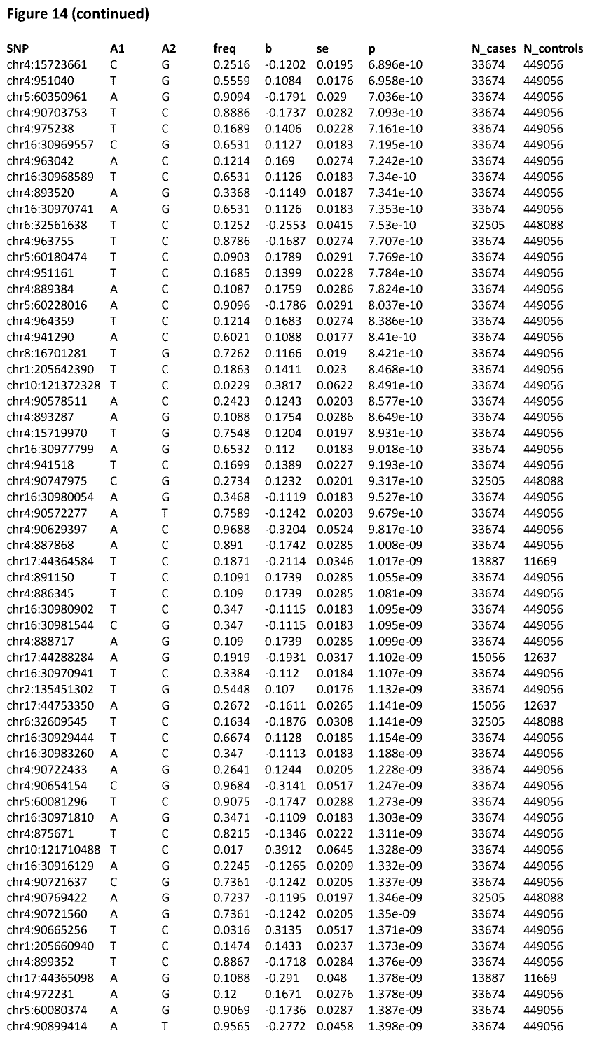

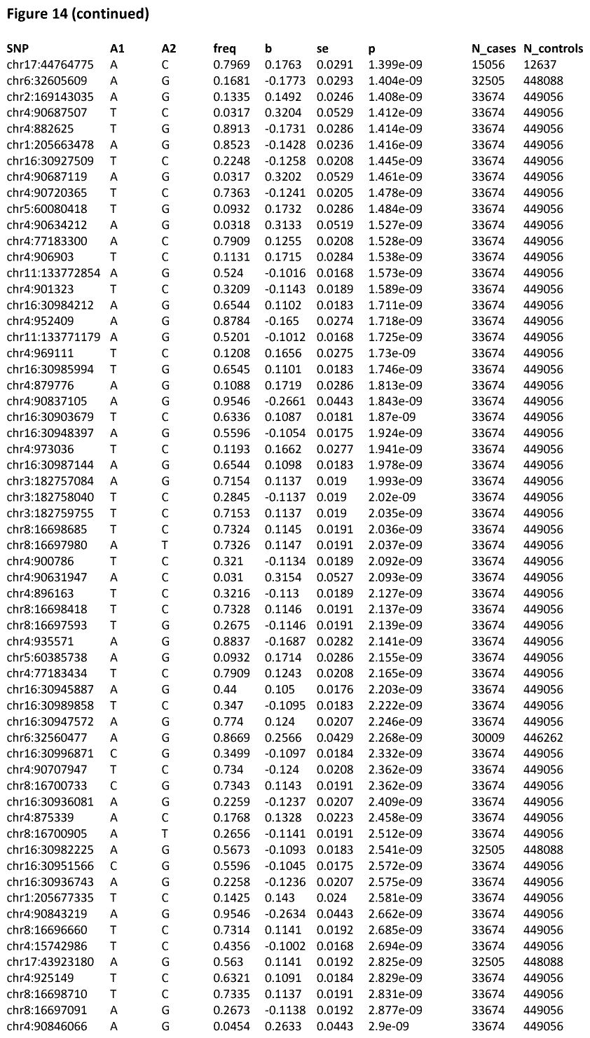

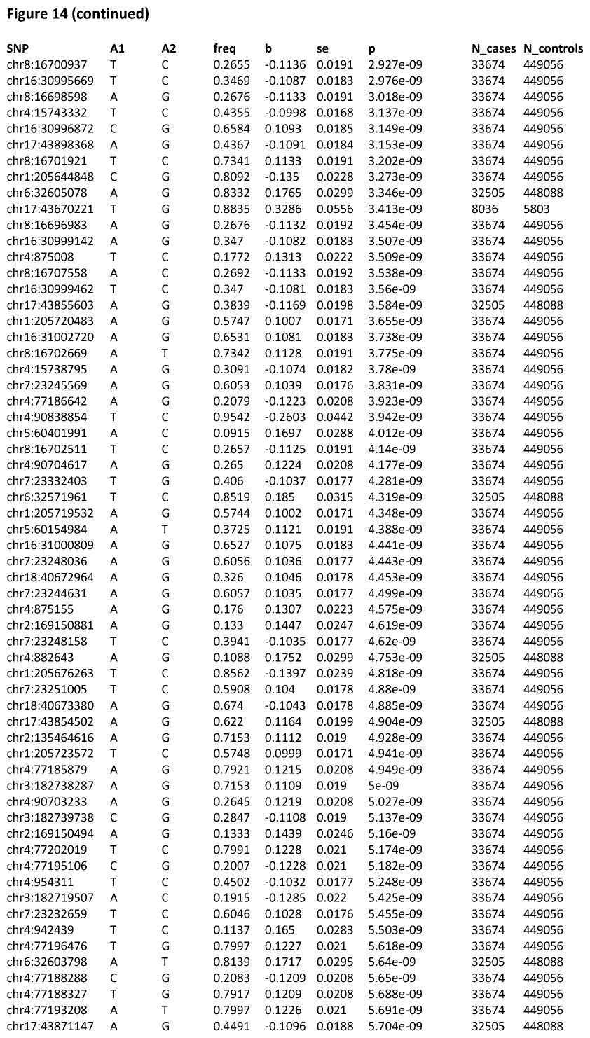

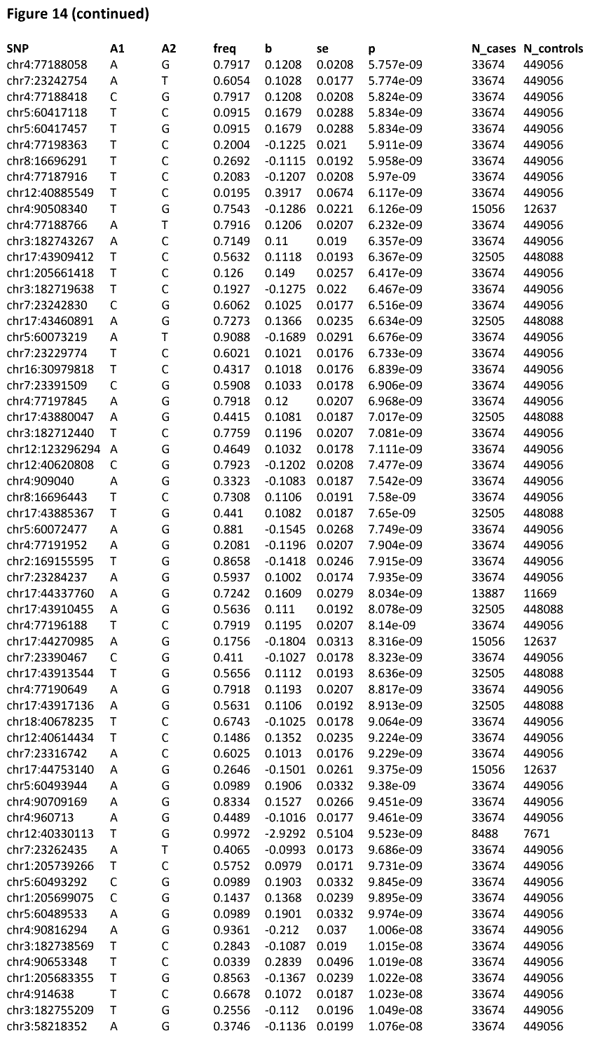

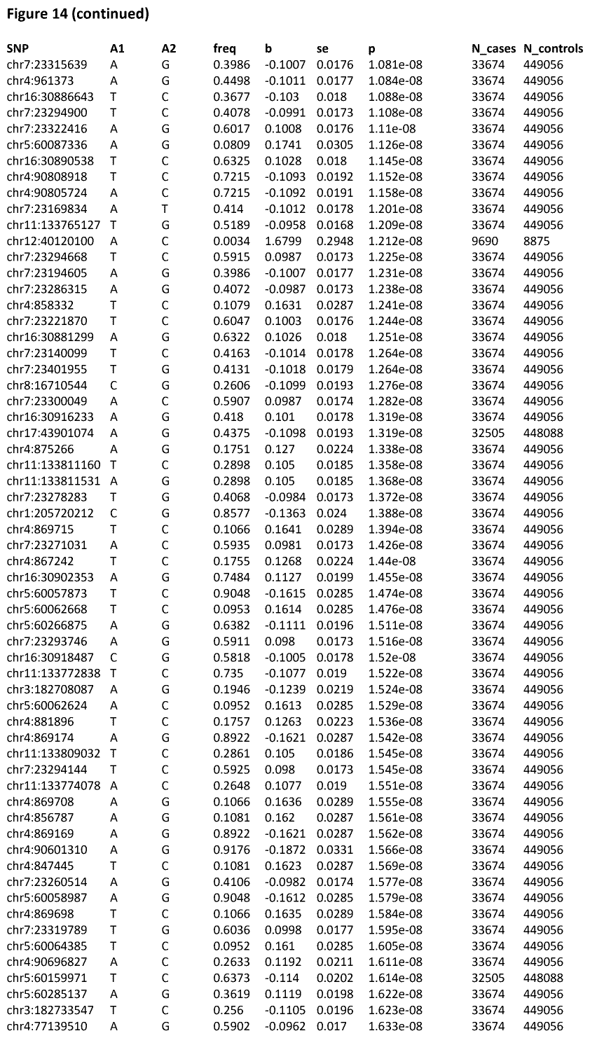

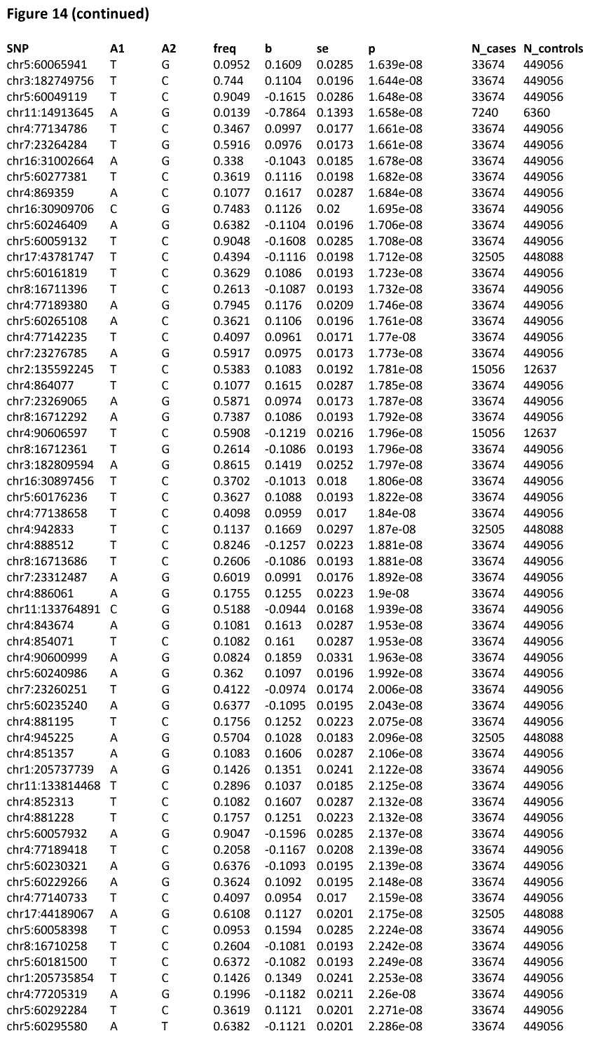

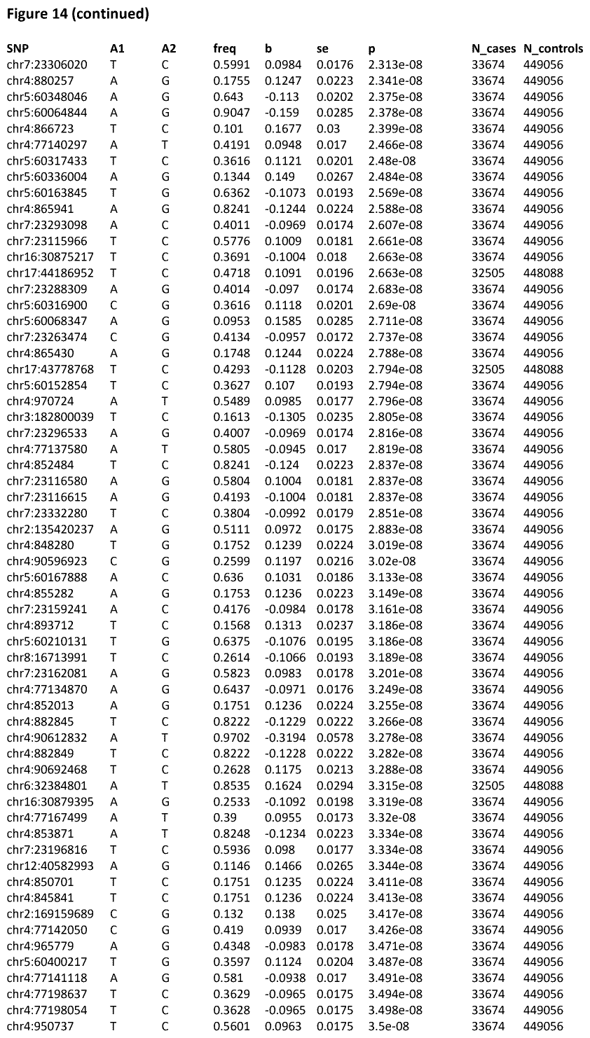

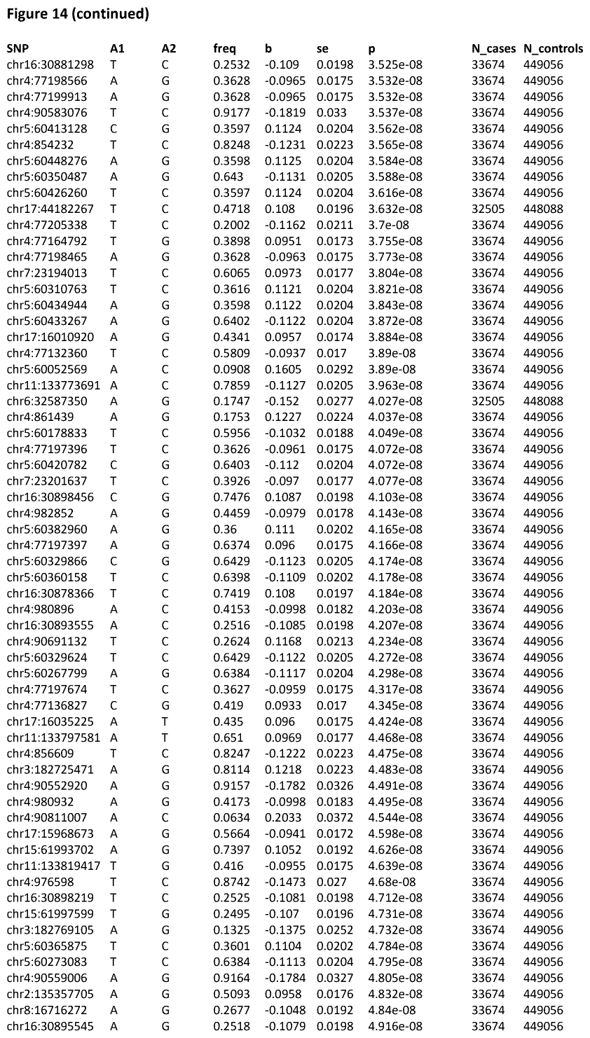

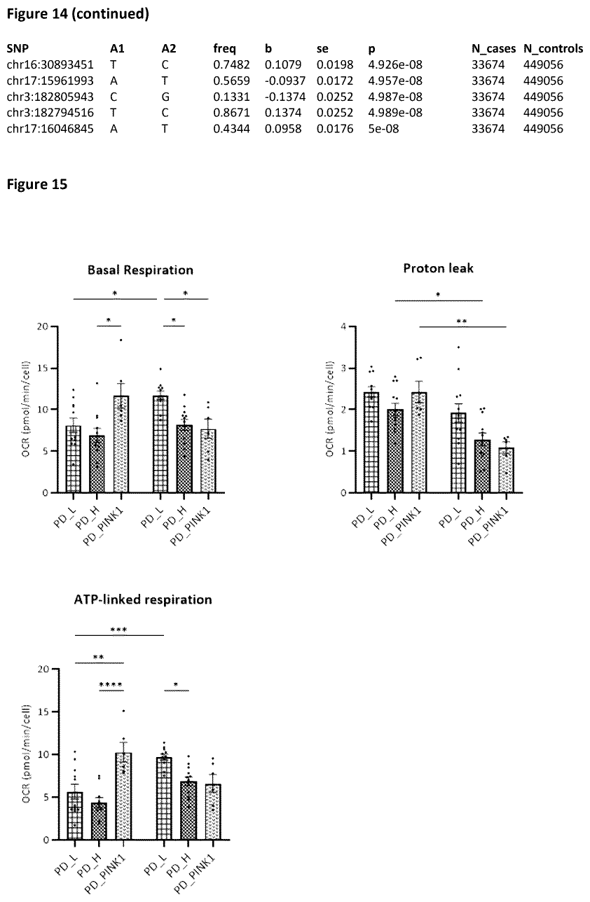

As disclosed herein, the OXPHOS-mito polygenic risk score can be calculated (obtained) using GWAS data for Parkinson's Disease. The largest summary statistics of PD GWAS to date is published by Nalls et al., 2019. Accordingly, in a preferred embodiment, the OXPHOS-mito polygenic risk score is obtained using GWAS data (in particular the summary statistic data) as published by Nalls et al. Lancet Neurol. 2019 December; 18 (12): 1091-1102 doi: 10.1016/S1474-4422(19)30320-5. For obtaining the an OXPHOS-mito polygenic risk score for the OXPHOS-mito genes listed in Table 9, corresponding SNPs as published by Nalls et al., 2019 can be used. Preferred SNPs are shown in FIG. 14, in particular in the first column.

Alternatively, but even less preferred, the OXPHOS-mito polygenic risk score can be calculated (obtained) using GWAS data from the LuxPark dataset (Hipp et al., 2018 Front. Aging Neurosci. 10, 326. https://doi.org/10.3389/fnagi.2018.00326).

Alternatively, but even less preferred, the OXPHOS-mito polygenic risk score can also be calculated (obtained) using GWAS data from the COURAGE-PD dataset (Grover et al., 2021, Mov Disord 36, 1689-1695. https://doi.org/10.1002/mds.28546).

A high OXPHOS-mito polygenic risk score refers to a value that is higher than the median value of a reference group. Said reference group is preferably a (preferably essentially unbiased) reference group reflecting a normal population. Said reference group can e.g. be the (entire) LuxPark cohort. Alternatively, the reference group can be the (entire) COURAGE-PD cohort. Preferably, a high OXPHOS-mito polygenic risk score refers to a value that is within the highest quartile of the refence group. Preferably, a high OXPHOS-mito polygenic risk score refers to a value that is within the highest 20% of the reference group. Preferably, a high OXPHOS-mito polygenic risk score refers to a value that is within the highest 15% of the reference group. More preferably, a high OXPHOS-mito polygenic risk score refers to a value that is within the highest 10% of the reference group.

A low OXPHOS-mito polygenic risk score refers to a value that is lower than the median value of a reference group. Said reference group is preferably a (preferably essentially unbiased) reference group reflecting a normal population. Said reference group can e.g. be the (entire) LuxPark cohort. Alternatively, the reference group can be the (entire) COURAGE-PD cohort. Preferably, a low OXPHOS-mito polygenic risk score refers to a value that is within the lowest quartile of the refence group. Preferably, a low OXPHOS-mito polygenic risk score refers to a value that is within the lowest 20% of the reference group. Preferably, a high OXPHOS-mito polygenic risk score refers to a value that is within the lowest 15% of the reference group. More preferably, a high OXPHOS-mito polygenic risk score refers to a value that is within the lowest 10% of the reference group.

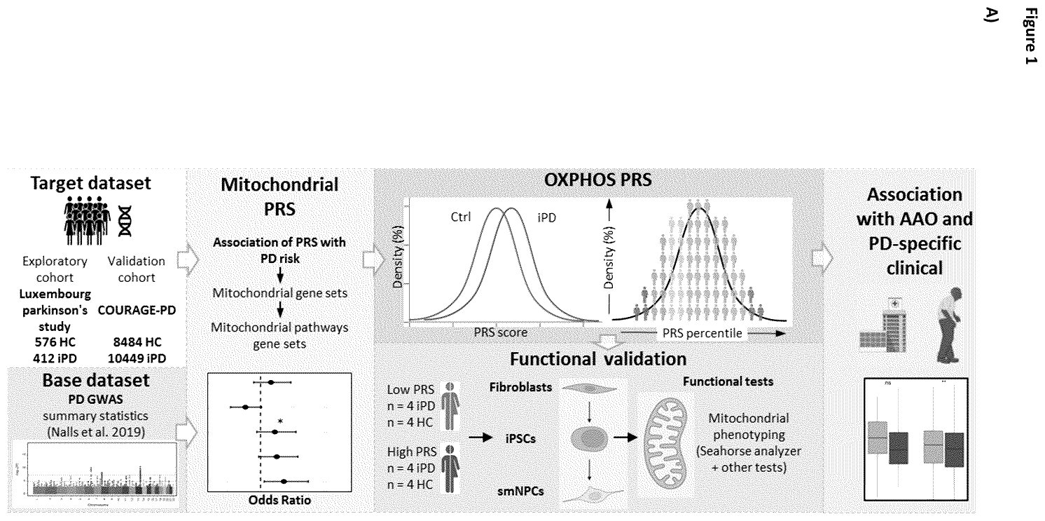

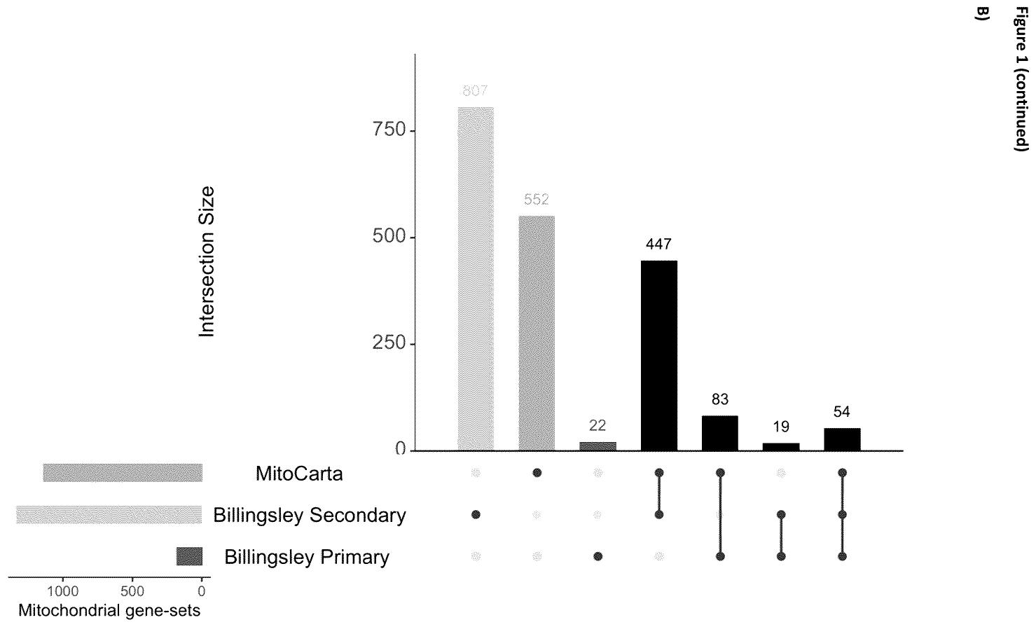

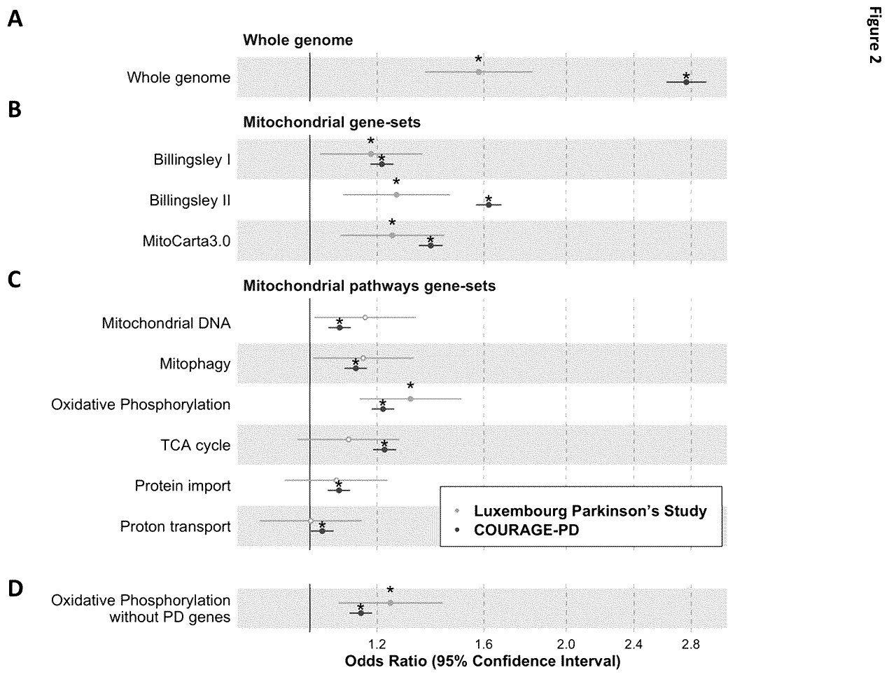

FIG. 1: (A) Study design and workflow. (B) Overlap between mitochondrial gene sets: Upset plot showing size (left) and intersection (right) of three mitochondrial gene-sets: Human MitoCarta3.0, Billingsley Primary and Billingsley Secondary, the latter taken from Billingsley et al.

FIG. 2: Common variants in mitochondrial genes were associated with higher PD risk. Forest plots of the odds ratio (OR) and 95% confidence interval for (A) whole genome, (B) three different mitochondrial gene-sets, (C) six different mitochondrial pathways polygenic risk scores (PRS) regressed with PD diagnosis for LuxPark, and for (D) Oxidative Phosphorylation pathway without the PD genes PINK1, SNCA and DJ-1. *: p<0.05 and thus significant FDR-adjusted p-value. Per line, upper, lighter depicted Forest plots are based on data obtained from the Luxembourg Parkinson's study; lower, darker Forest plots are based on data obtained from the COURAGE-PD data set.

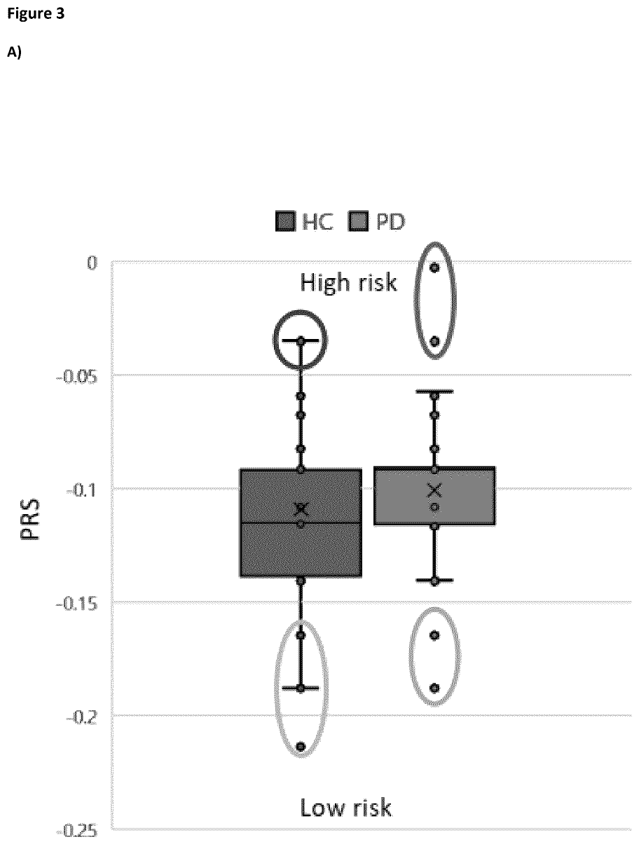

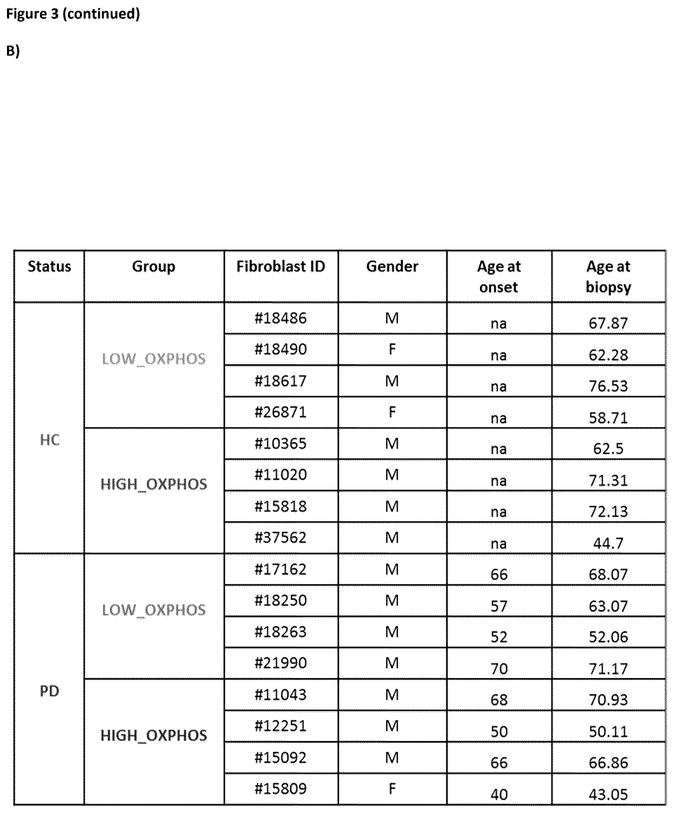

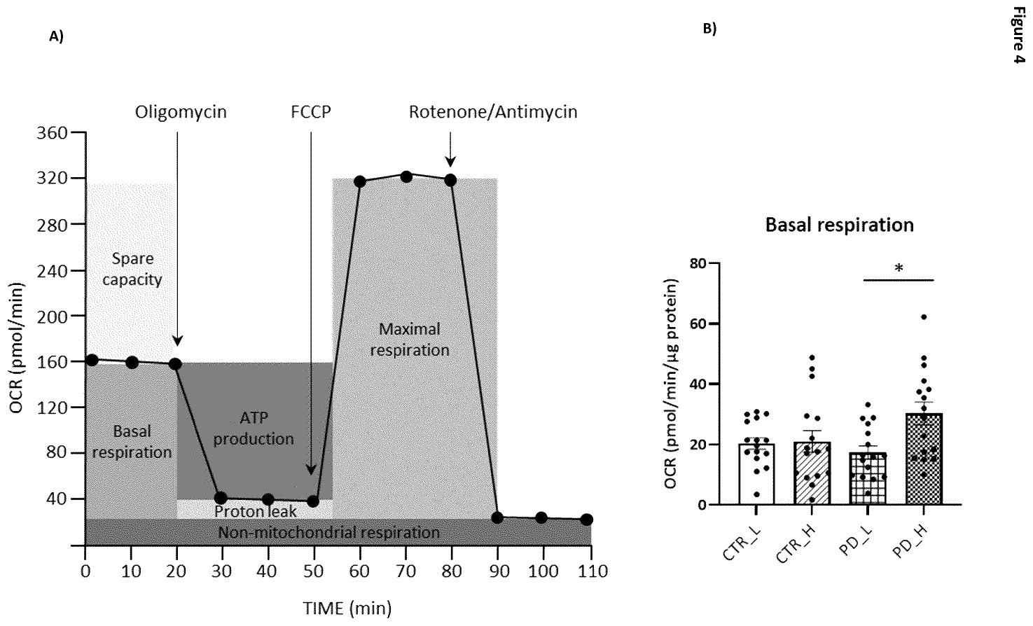

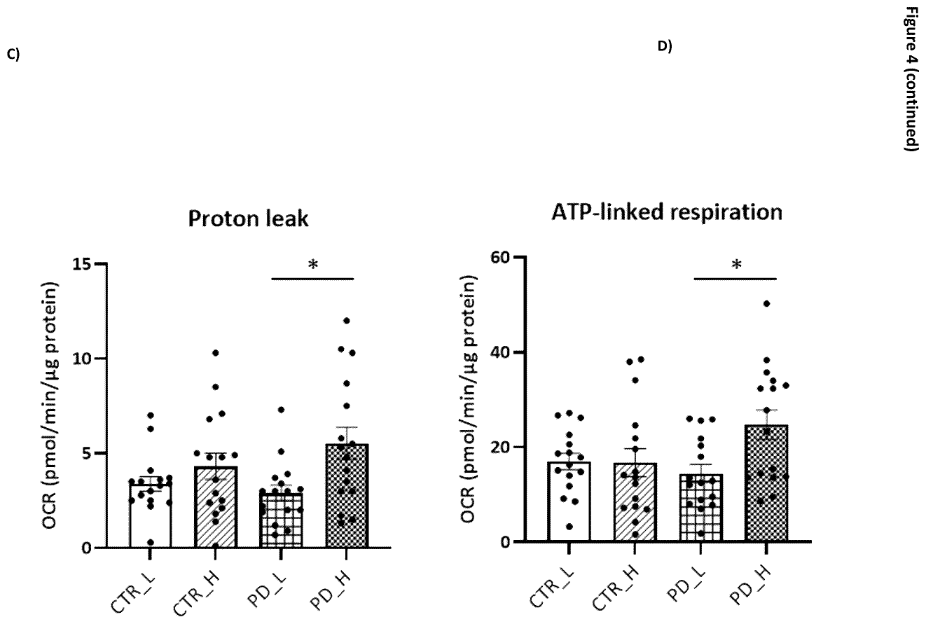

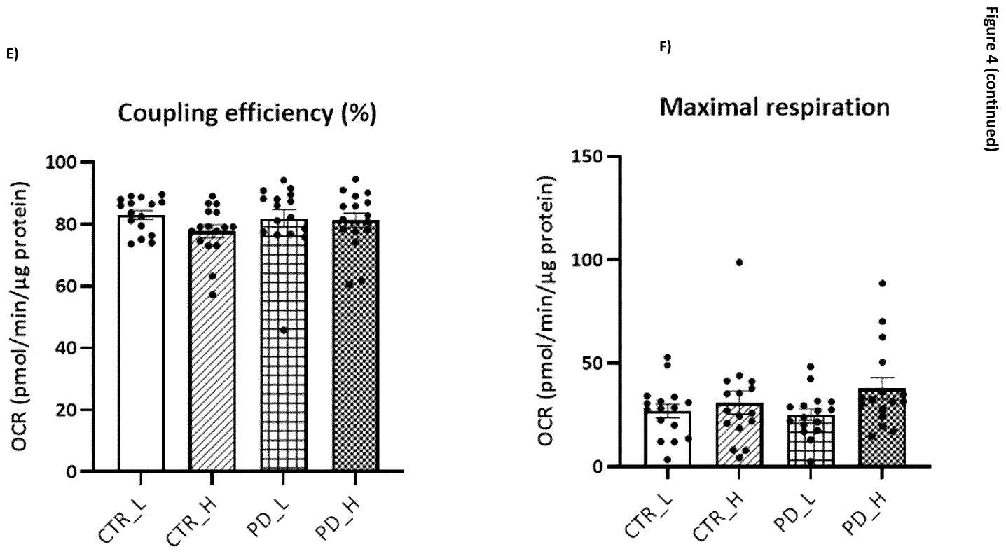

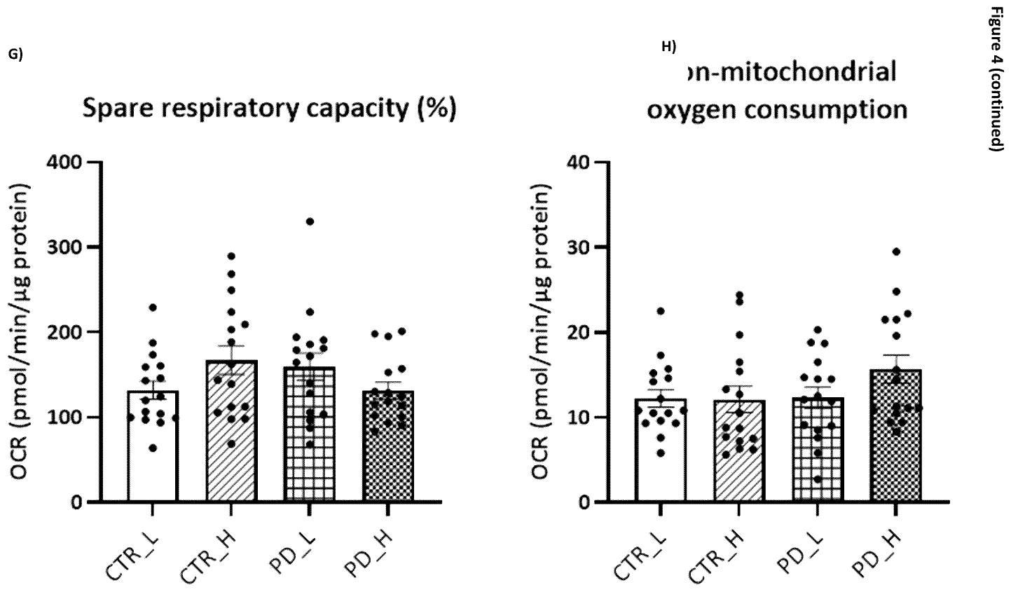

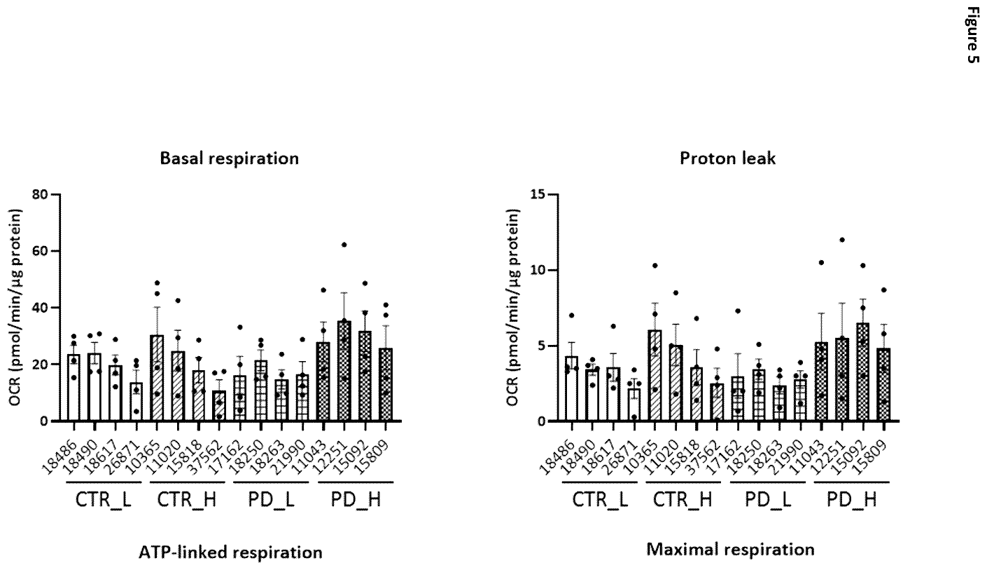

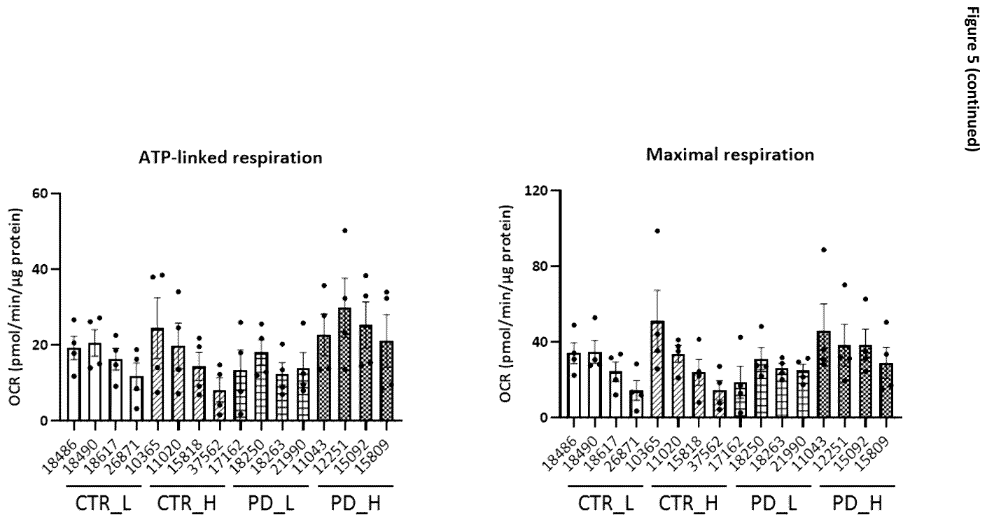

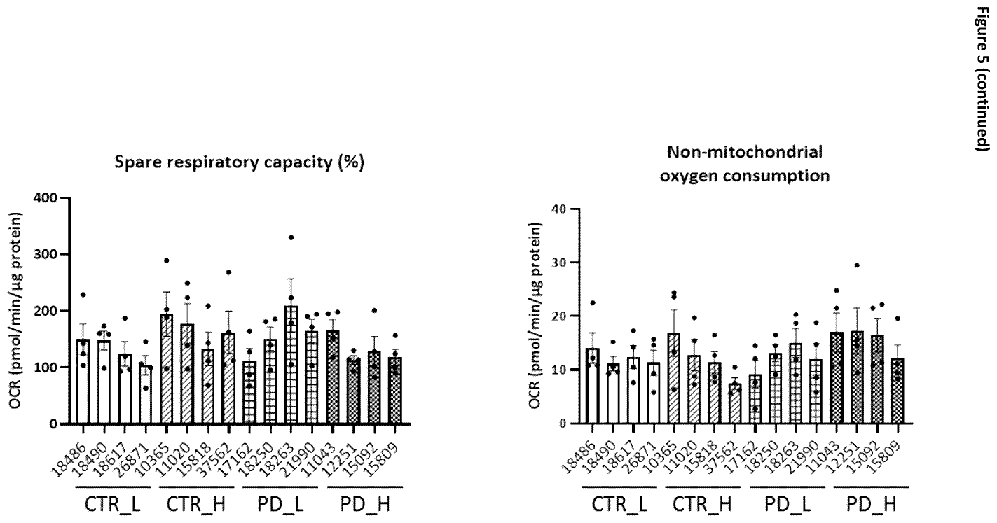

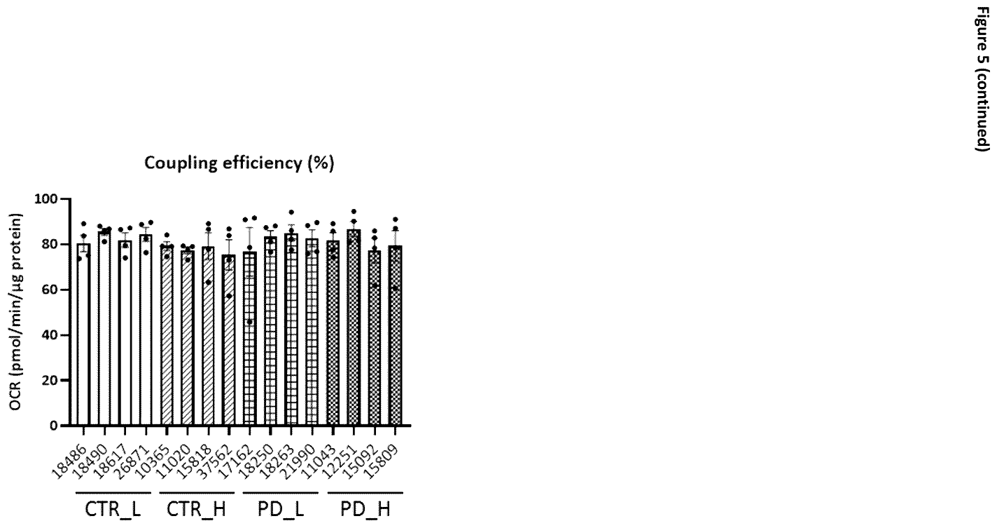

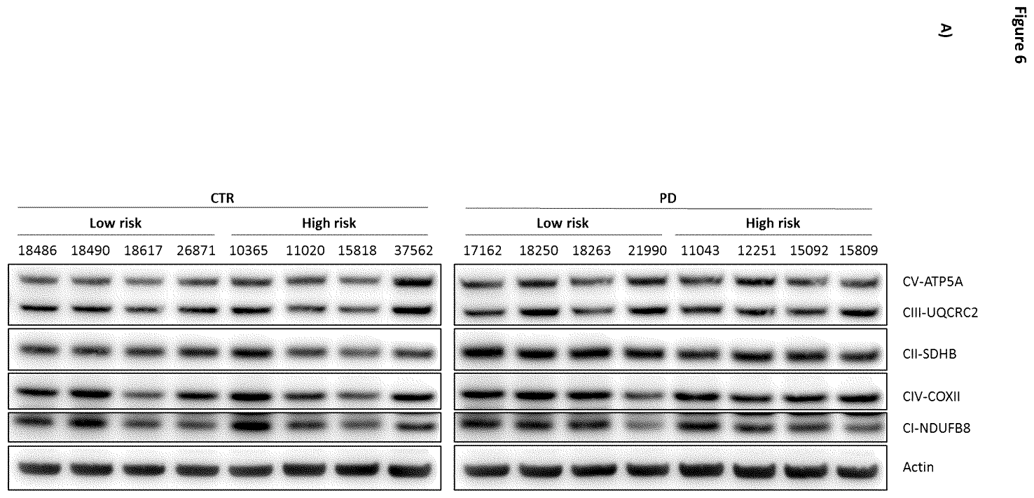

FIG. 3: (A) Boxplots of PRS for OXPHOS-mito pathway genes for healthy controls (HC; left) and PD patients (PD, right) from the LuxPark study with circles indicating patients with OXPHOS-mito PRSs within the 10th and 90th percentile per group, respectively, as identified in primary skin fibroblasts in the LuxPark cohort (n=4 per group). (B) Gender, age of PD patients at the onset of first symptoms and age of study participants at the time of skin biopsy.

FIG. 4: Analysis of mitochondrial respiration in primary skin fibroblasts from iPD patients and HC stratified based on OXPHOS-PRS. Enhanced mitochondrial respiration in primary skin fibroblasts from PD patients with high OXPHOS-mito PRS: (A) Oxygen consumption rate (OCR) profile over time. (bar plots B to H) OCR of different parameters related to oxidative phosphorylation of HC with low individual OXPHOS-mito PRS, HC with high individual OXPHOS-mito PRS, PD with low individual OXPHOS-mito PRS and PD with high individual OXPHOS-mito PRS (from left to right; n=4 per group; with high and low referring to the 10th and 90th percentile per HC and PD, respectively). In particular, oxygen consumption rates (OCRs) were measured under basal conditions and after targeted inhibition of specific respiratory chain complexes by using a standard Seahorse Mito Stress test. Histobars represent the pooled means of four independent experiments performed in four distinct fibroblast lines established from Luxembourg Parkinson's Study participants (healthy controls vs iPD patients) with high (CTR_H; PD_H) or low (CTR_L; PD_L) OXPHOS-PRS. *p<0.05.

FIG. 5: Individual OCRs underlying the respective summary data shown in FIG. 4 (bar plots).

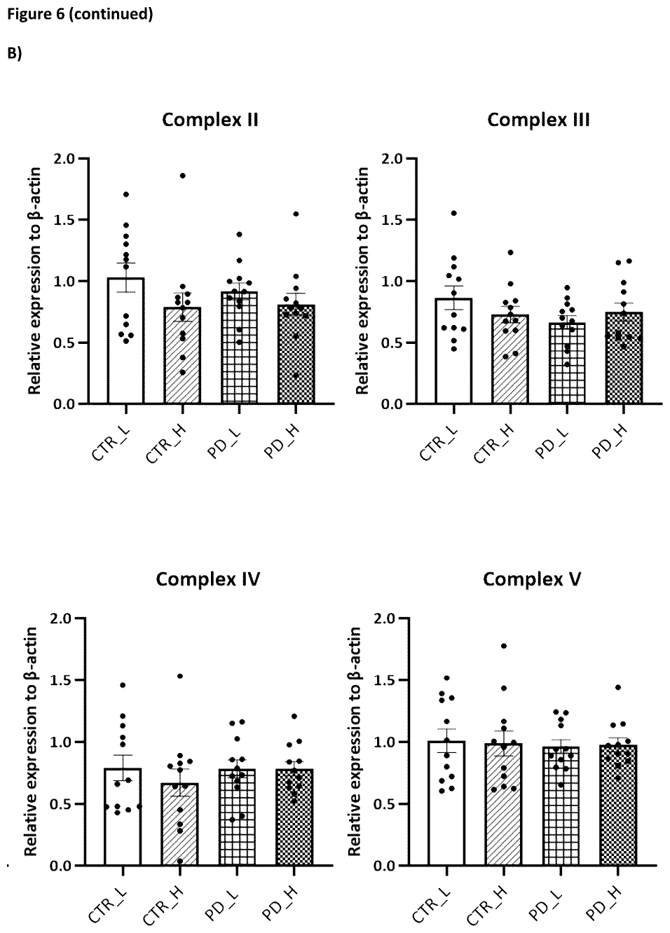

FIG. 6: (A) Immunoblot analysis of respiratory chain complex (RCC) protein subunits per individual for healthy controls (left) and PD (right) with characteristics of individuals being listed in Table 11. (B; box plots) Quantitative densitometric analysis of immunoblotting experiments as in (A: immunoblot data standardized with Actin levels and summarized per RCC protein subunit per group) with histobars representing pooled means of three independent experiments performed in four distinct fibroblast lines established from Luxembourg Parkinson's Study participants (healthy controls vs iPD patients; from left to right: HC with low individual OXPHOS-mito PRS, HC with high individual OXPHOS-mito PRS, PD with low individual OXPHOS-mito PRS and PD with high individual OXPHOS-mito PRS; n=4 per group; with high and low referring to the 10th and 90th percentile per HC and PD, respectively).

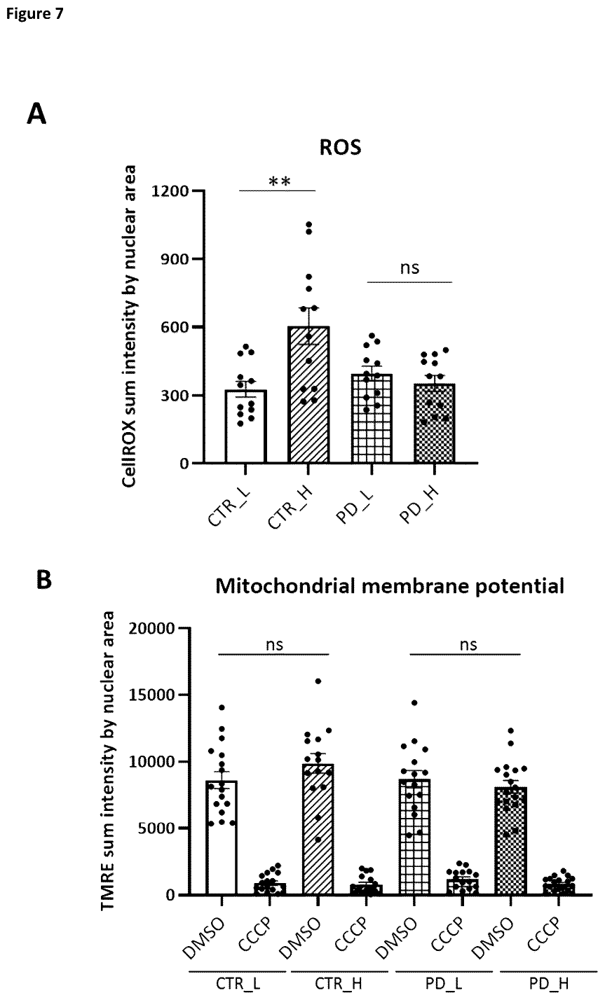

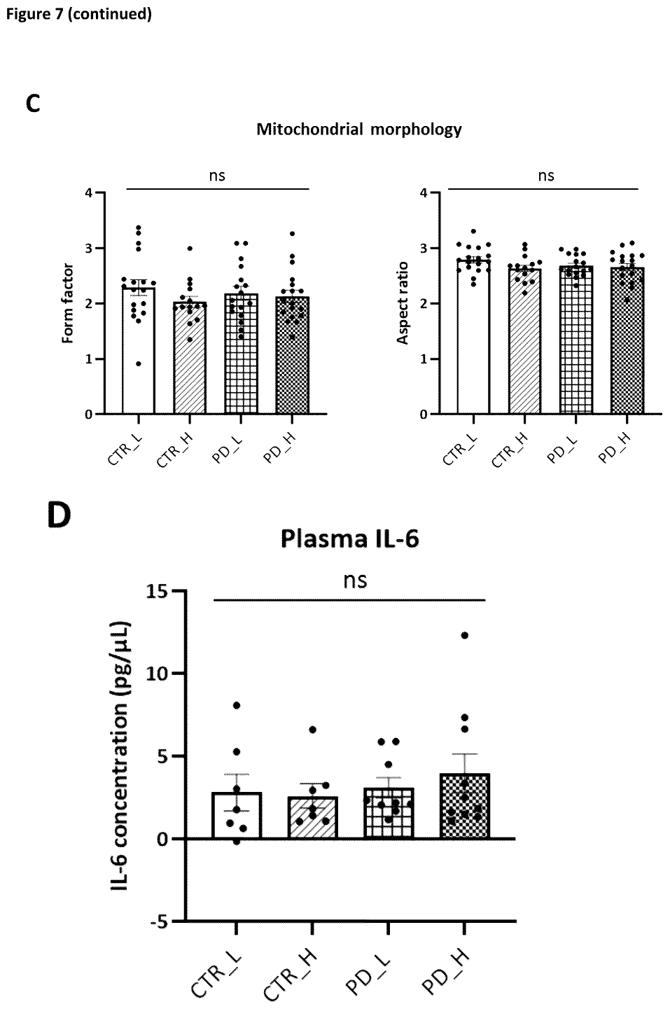

FIG. 7: Assessment of additional readouts of mitochondrial activity in primary skin fibroblasts and plasma samples from iPD patients and HC stratified based on OXPHOS-PRS. High-throughput confocal microscopy analyses in primary skin fibroblasts derived from Luxembourg Parkinson's Study participants with high (CTR_H; PD_H) or low (CTR_L; PD_L) OXPHOS-PRS. (A) Reactive oxygen species (ROS) levels quantified by normalizing the CellRox mean fluorescence intensity against the nuclear area, as defined by the Hoechst staining. Histobars represent the pooled means of three independent experiments performed in four distinct fibroblast lines for each group, (B) mitochondrial membrane potential (Δψm) was measured after normalization of TMRE mean fluorescence intensity by the nuclear area, treatment with the OXPHOS uncoupler carbonyl cyanide 3-chlorophenylhydrazone (CCCP), known to induce mitochondrial depolarization, was used as positive control for decreased Δψm. Dimethyl sulfoxide (DMSO) was used as a vehicle. Histobars represent the pooled means of at least three independent experiments performed in four distinct fibroblast lines for each group, (C) mitochondrial morphology and thus, morphometric analysis of the mitochondrial network, form factor and aspect ratio were quantified as described previously, Histobars represent the pooled means of at least three independent experiments performed in four distinct fibroblast lines for each group, and (D) individual IL-6 plasma concentrations obtained from Luxembourg Parkinson's Study participants of HC with low individual OXPHOS-mito PRS, HC with high individual OXPHOS-mito PRS, PD with low individual OXPHOS-mito PRS and PD with high individual OXPHOS-mito PRS (from left to right; n=4 per group; with high and low referring to the 10th and 90th percentile per HC and PD, respectively).

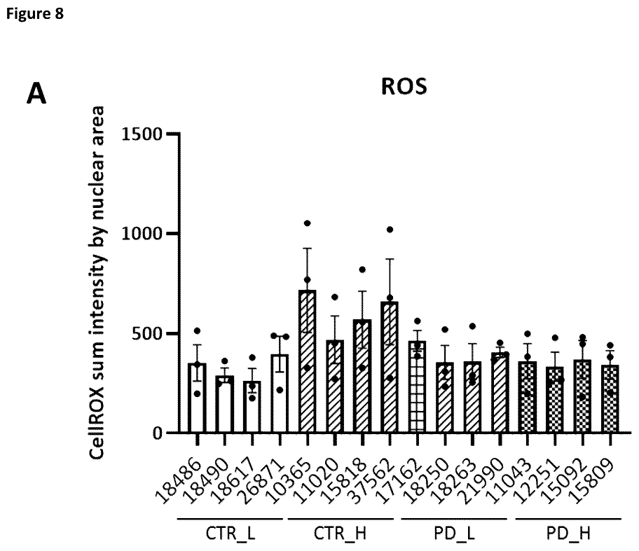

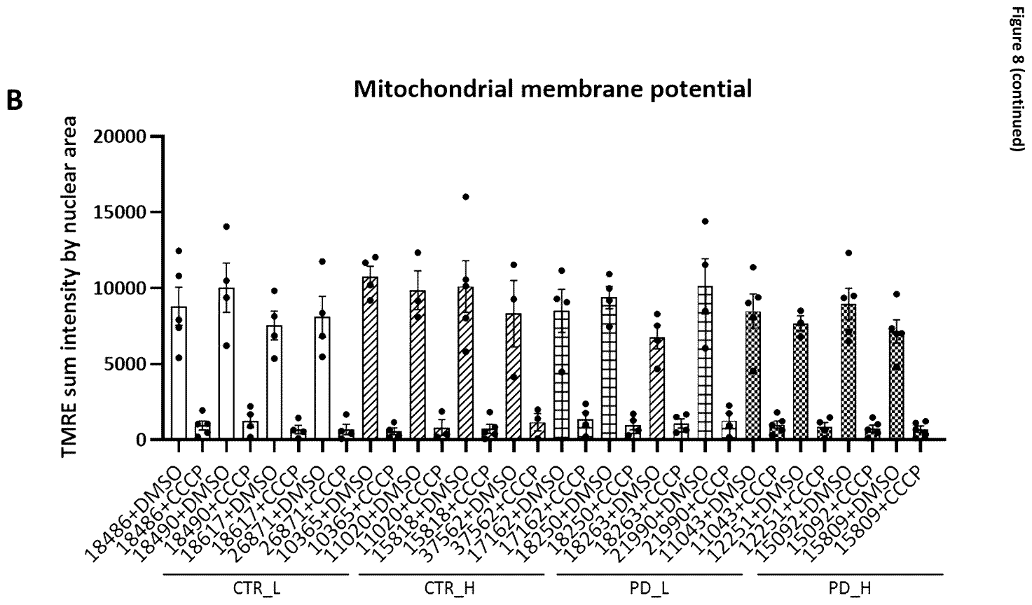

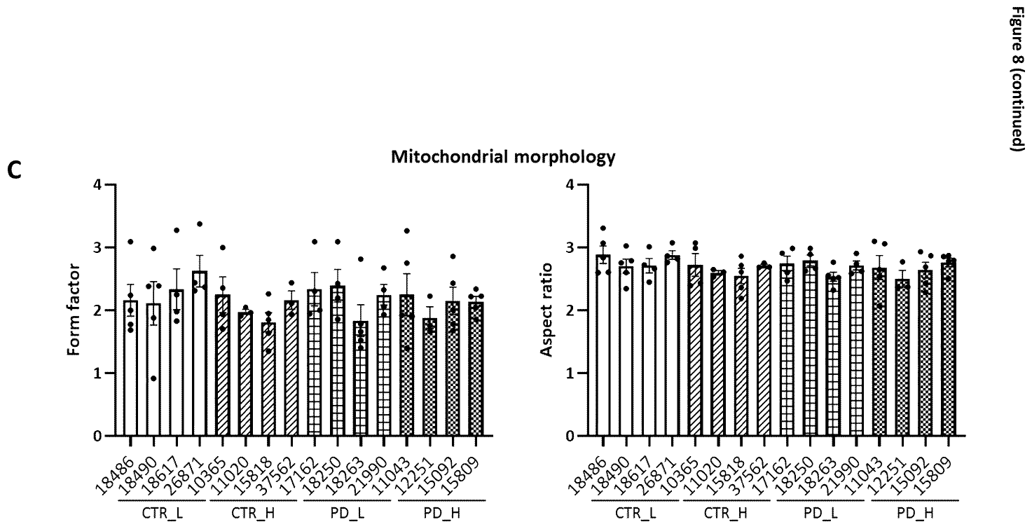

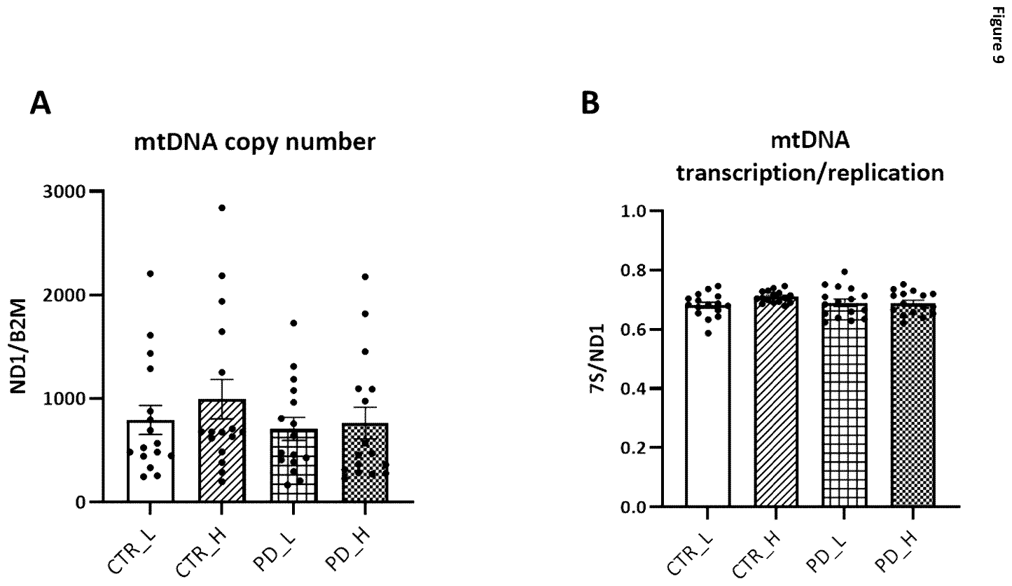

FIG. 8: (A) to (C) Individual data underlying the respective summary data shown in FIG. 7 (A) to (C).

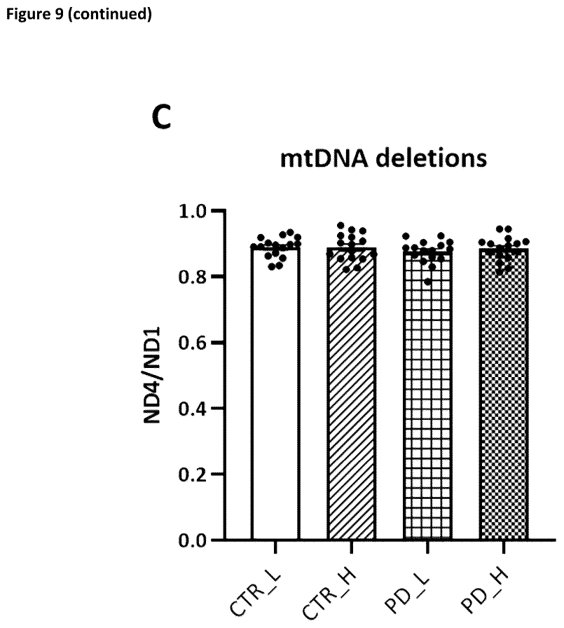

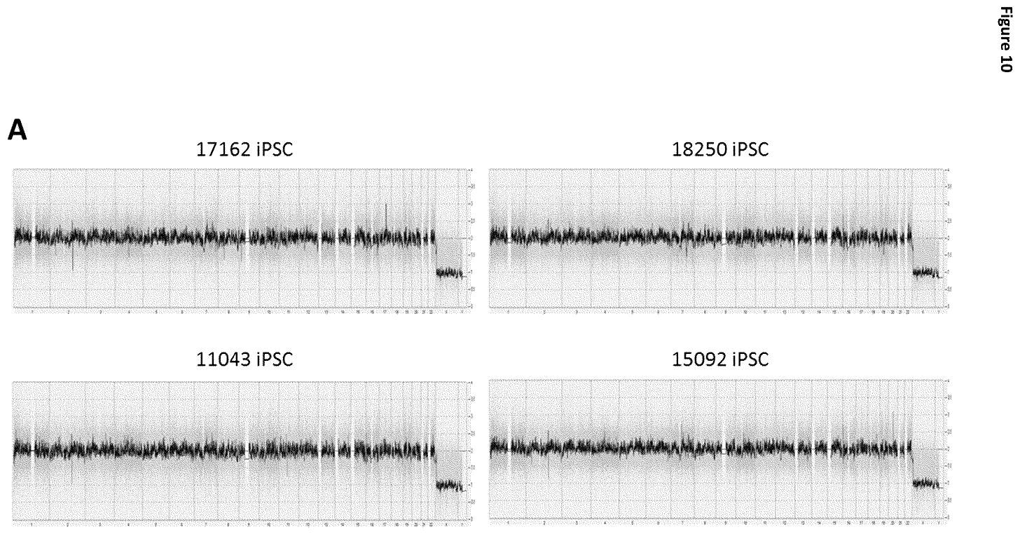

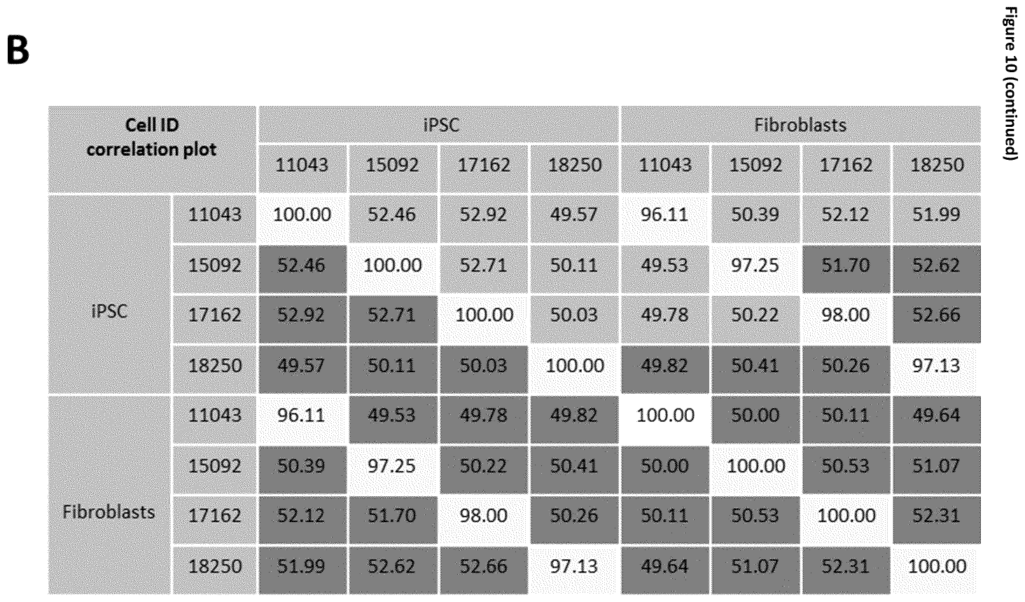

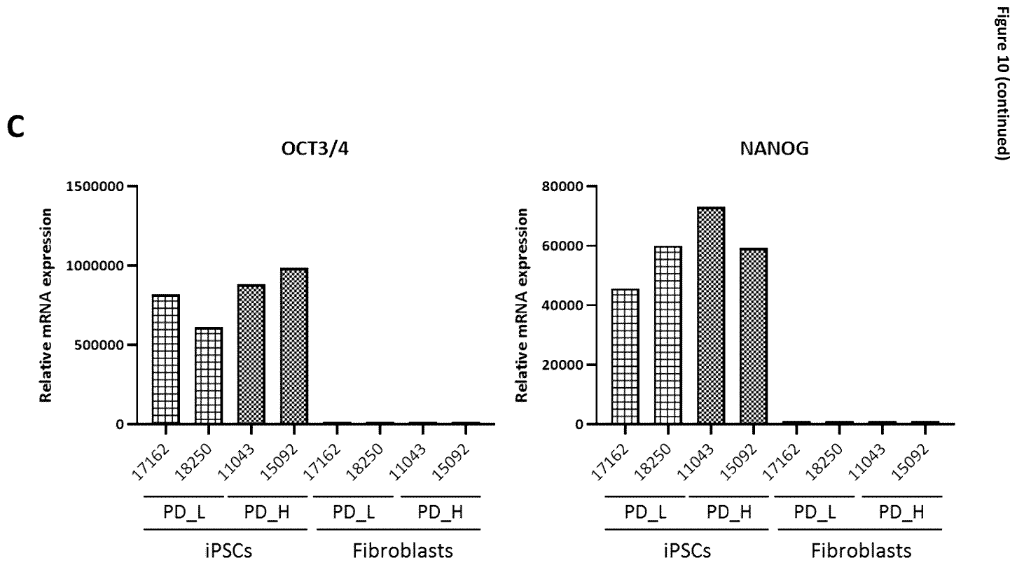

FIG. 9: Evaluation of mtDNA integrity by triplex qPCR assay. (A) Number of copies of mtDNA assessed by calculating the ratio of ND1 to B2M. (B) Transcription/replication status of mtDNA assessed by the ratio of 7S DNA over ND1. (C) Level of major arc deletions in the mitochondrial genome assessed by calculating the ND4 to ND1 ratio. (A) to (C) are shown per group, i.e. HC with low individual OXPHOS-mito PRS, HC with high individual OXPHOS-mito PRS, PD with low individual OXPHOS-mito PRS and PD with high individual OXPHOS-mito PRS (from left to right; with high and low referring to the 10th and 90th percentile per HC and PD, respectively). Histobars represent the mean of 4 independent experiments with 4 distinct fibroblast lines from healthy controls and iPD patients with high or low OXPHOS-mito PRSs. *p<0.05.

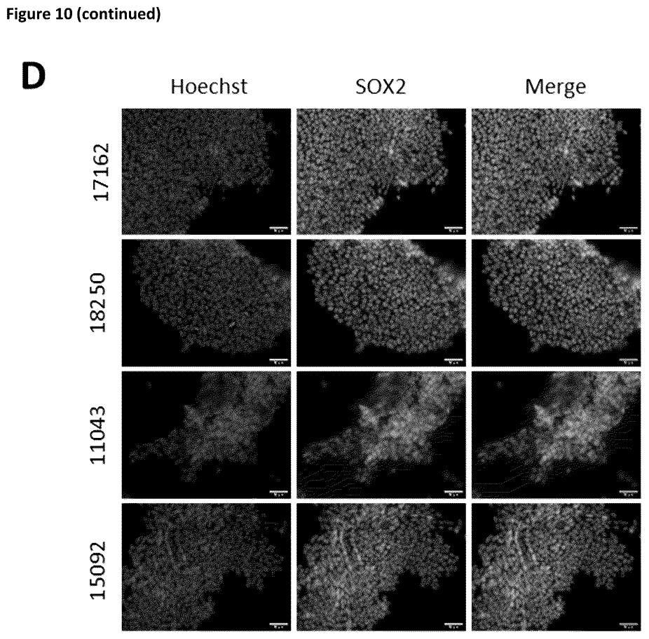

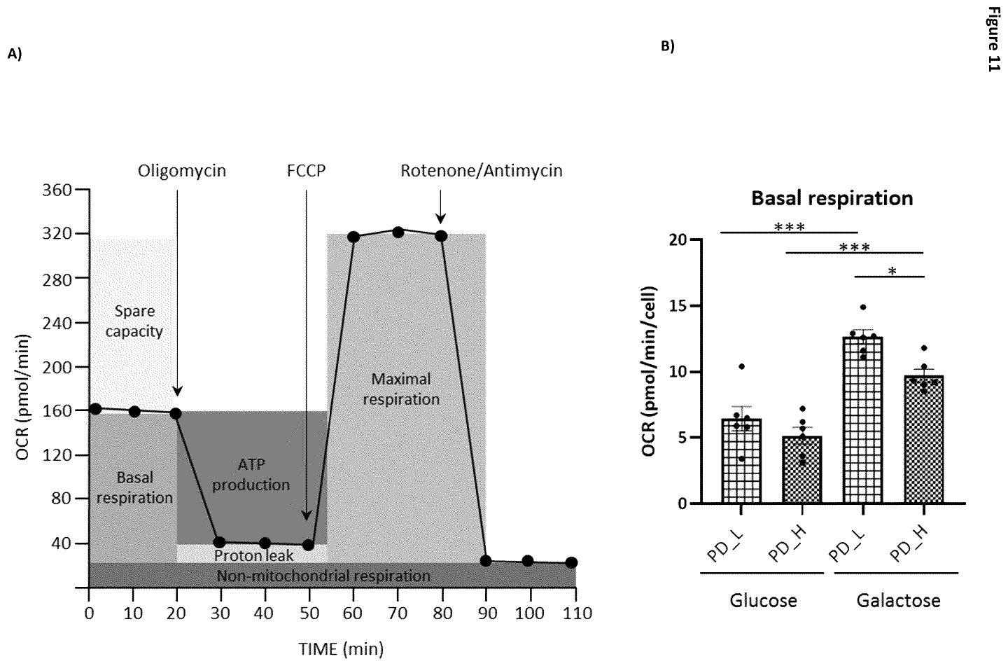

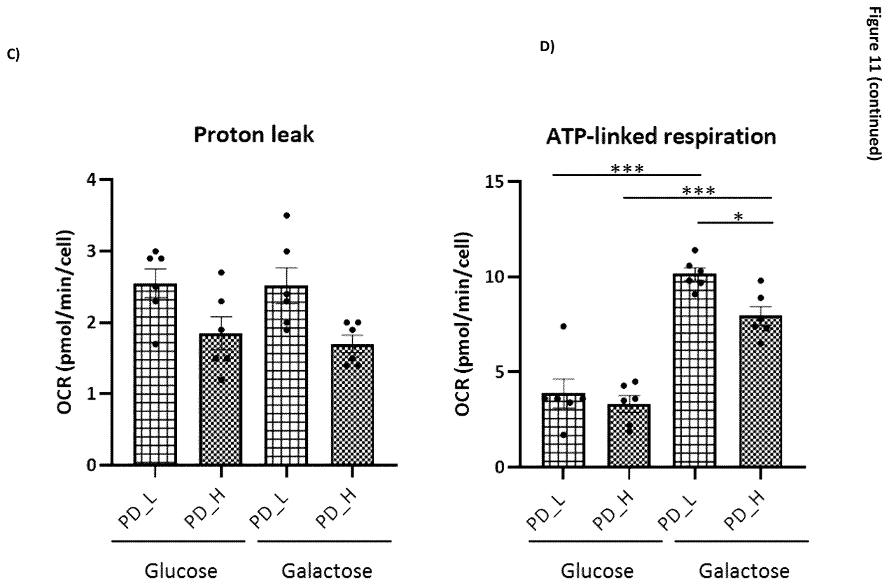

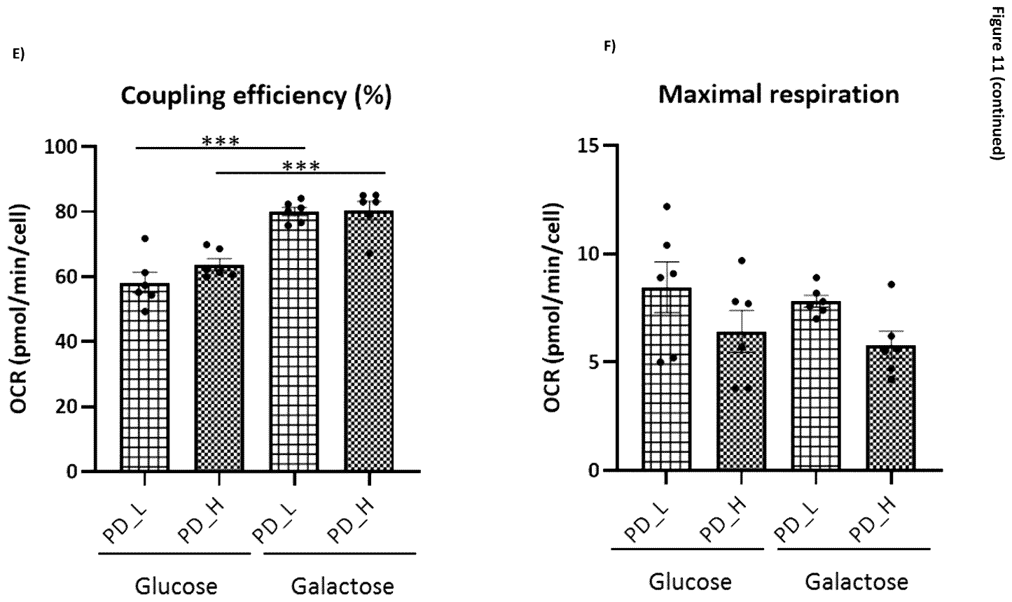

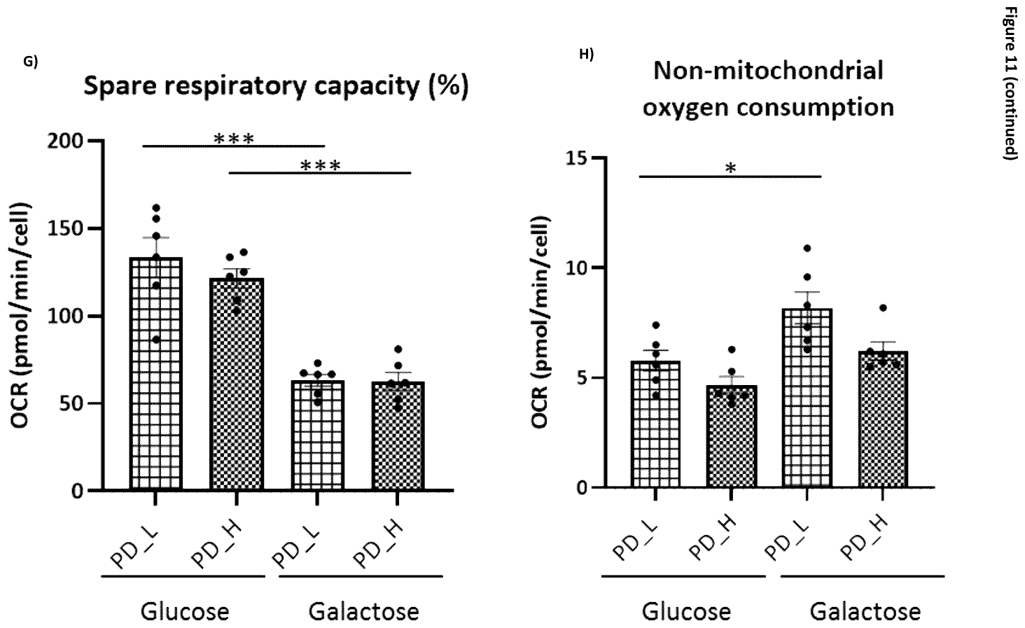

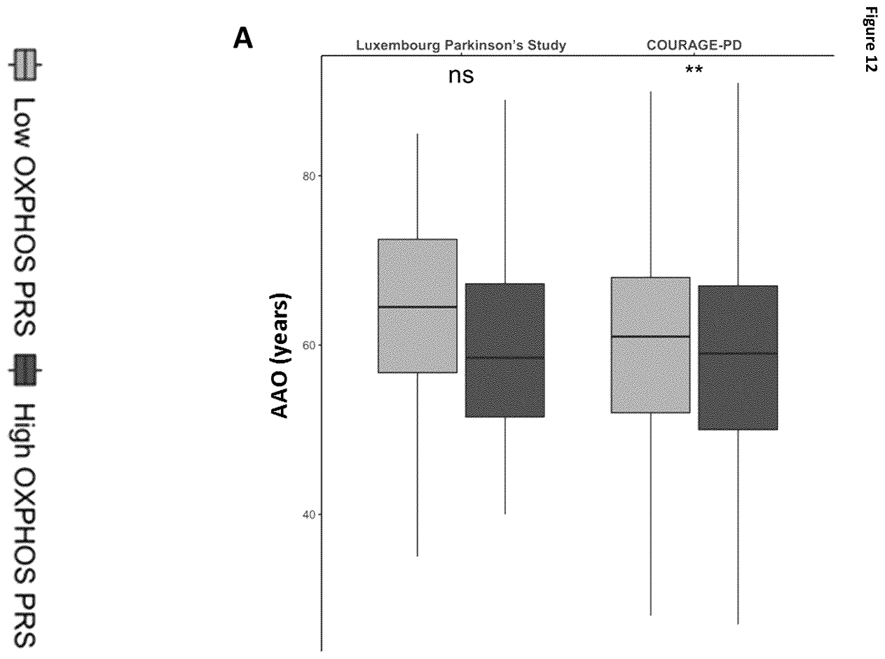

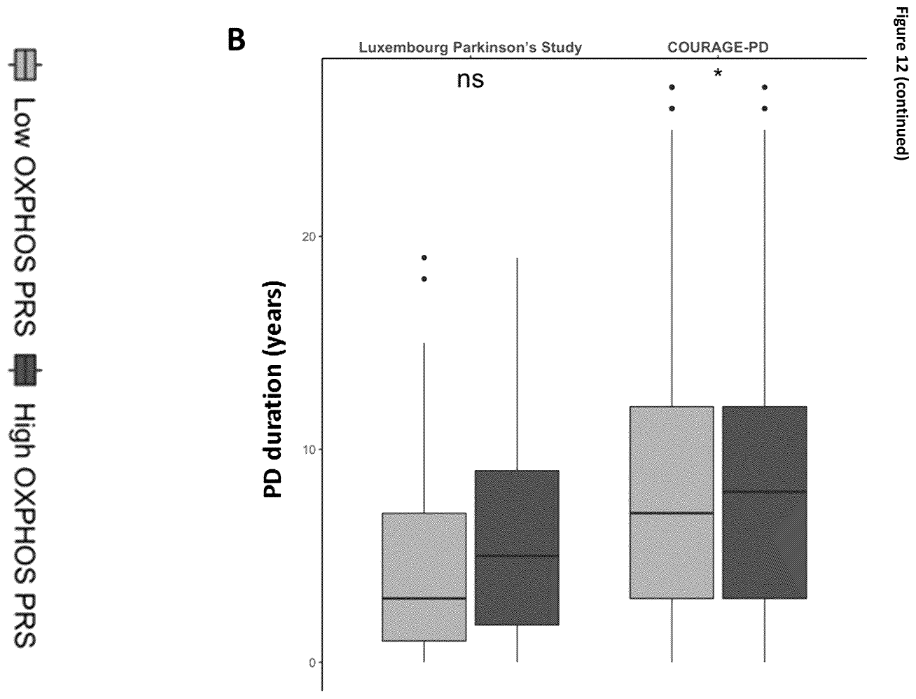

FIG. 10: Successfully reprogrammed into iPSCs (n=2 per group) of PD patients within the 10th (low) and 90th percentile (high) based on OXPHOS-mito PRS (#17162 and #18250: low OXPHOS-mito PRS; #11043 and #15092: high OXPHOS-mito PRS): (A) No chromosomal aberrations and derived from male individuals as expected (Table 11). Array-based karyotyping of iPSC lines. Somatic and sex chromosomes are represented in the whole genome view. The smooth signal plot (right y-axis) shows the smoothing of the log 2 ratios which depict the signal intensities of probes on the microarray. (B) Correlation analysis of 150k SNPs spread across the genome of iPSC clones vs. the original skin fibroblasts. The correlation between a sample and itself has a value of 100, correlations >95% between samples are considered having an identical genetic background (green boxes), correlations <95% indicate different genetic backgrounds (red boxes). (C) RNA expression levels of the stemness markers OCT3/4 and NANOG: RT-qPCR analysis of the stemness markers OCT3/4 and NANOG in iPSCs and primary skin fibroblasts. OCT3/4 and NANOG mRNA expression levels were normalized against ACTB. (D) Expression confirmation of the stem cell marker SOX2 by immunofluorescence: Immunofluorescence analysis of the stem cell-specific transcription factor SOX2 in the newly generated iPSC lines. Cells expressing SOX2 were stained in green. Nuclei were stained with Hoechst.

FIG. 11: Functional validation of OXPHOS-PRS in iPSC-derived neuronal progenitor cells. smNPCs derived from PD patients with low and high OXPHOS-mito PRS (n=2 per group; with high and low referring to the 10th and 90th percentile within PD, respectively): (A) Oxygen consumption rate (OCR) profile over time. (bar plots B to H) OCR of different parameters related to oxidative phosphorylation of smNPCs in either glucose (left) or galactose (right) medium, respectively. In particular, oxygen consumption rates (OCRs) were measured under basal conditions and after targeted inhibition of specific respiratory chain complexes by using a standard Seahorse Mito Stress test. Histobars represent the pooled means of three independent experiments performed in two distinct smNPC lines established from iPD patients high (PD_H) or low (PD_L) OXPHOS-PRS, cultivated in glucose or galactose medium. *p<0.05, ***p<0.001.

FIG. 12: Association of OXPHOS-PRSs with PD-specific clinical outcomes. Individuals with high OXPHOS-PRS tended to have significant earlier AAO and longer disease duration compared to low-risk patients (p<0.05). Comparison of AAO (A) and disease duration (B) in iPD patients from the COURAGE-PD (right) cohorts and the Luxembourg Parkinson's study (left) with high or low OXPHOS-PRS. *p<0.05, **p<0.01, ns=not significant.

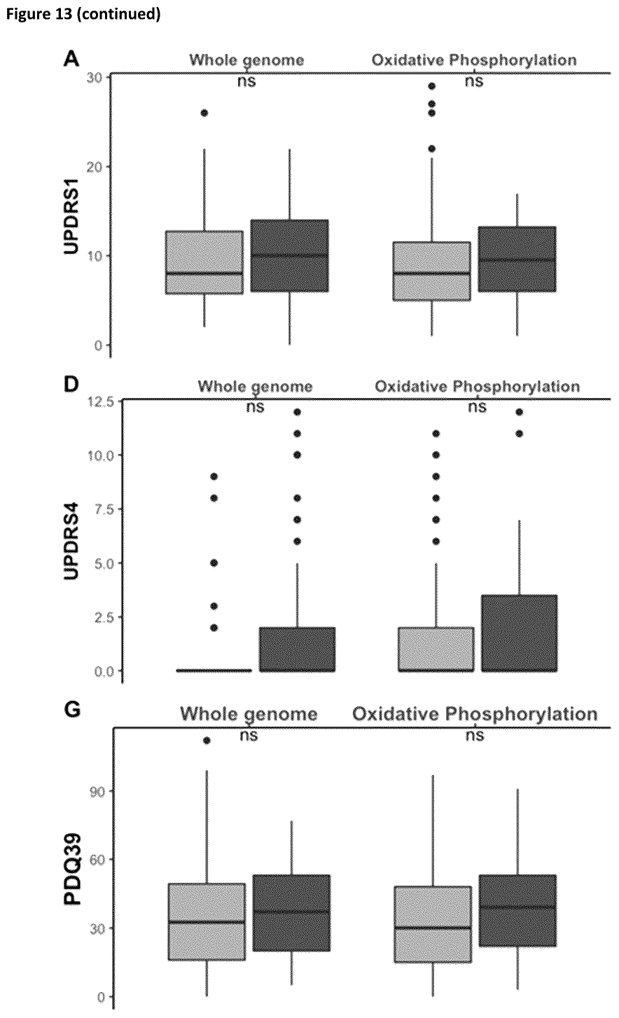

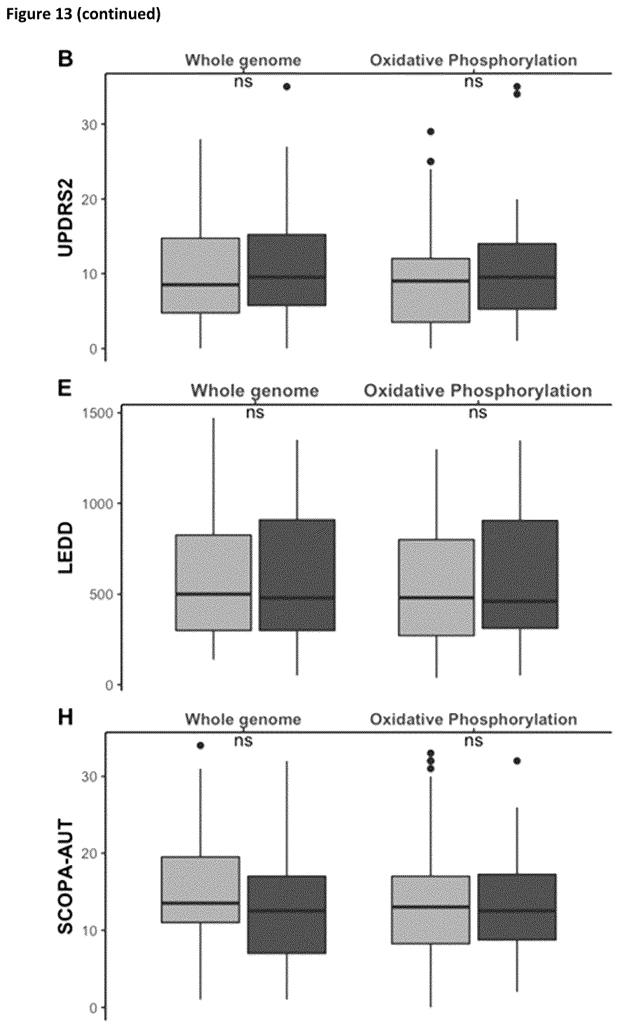

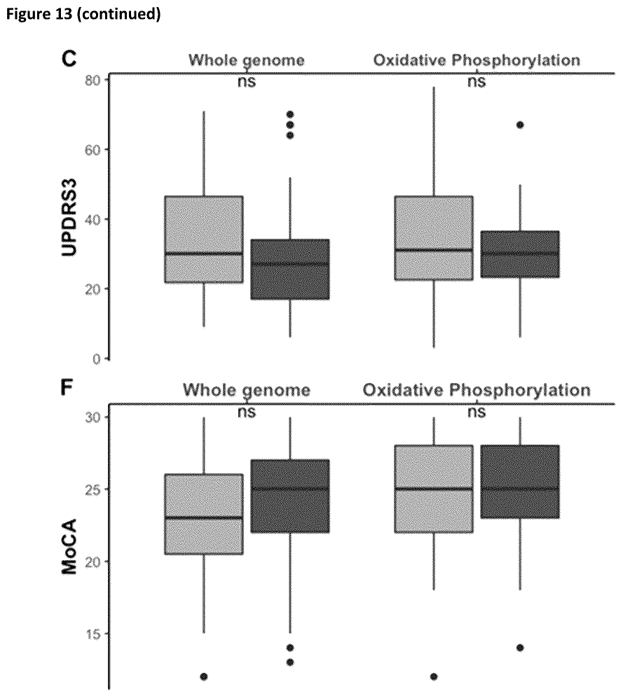

FIG. 13: Individuals with high OXPHOS-PRS showed a worse quality of life (as defined by higher PDQ-39), but none of the shown clinical outcome scores reached statistical significance (“ns”).

FIG. 14: List of preferred SNPs with information as regards chromosomal position, alleles, effect size, likelihood and sample sizes. SNPs relate to the 124 genes of the OXPHOS pathway. In particular, given are the position of the respective bi-allelic SNP (“SNP”), its two observed alleles “A1” and “A2”, allele frequency (“freq”), effect size reported as beta value (“b”), standard error (“se”), significance of association (“p”) and sample sizes for PD (“N_cases”) and healthy controls (“N_controls”).

FIG. 15: Oxygen consumption rates (OCRs) comparison between smNPCs derived from PD patients with low or high OXPHOS-mito PRS and PINK1-mutant smNPCs. OCRs were measured under basal conditions and after targeted inhibition of specific respiratory chain complexes using a standard Seahorse Mito Stress test. Histobars represent the pooled mean±SEM of either 6 (PD_L and PD_H) or 3 (PD_PINK1) independent experiments performed in two distinct cell lines per group, cultivated either in glucose or galactose medium. 8 technical replicates per group (distinct Seahorse wells) were performed in each experiment. *P<0.05; ** P<0.01; *** P<0.001; **** P<0.0001. 2-way ANOVA correcting for multiple comparisons using the Tukey's post hoc test.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms. E.g., the term “comprising” is meant to provide explicit support also for “consisting essentially of” and “consisting of”, the term “consisting essentially of” is meant to provide explicit support also for “comprising” and “consisting of”, the term “consisting of” is meant to provide explicit support also for “consisting essentially of” and “comprising”. Other aspects and advantages of the invention will be described in the following examples, which are given for purposes of illustration and not by way of limitation.

### Examples

Methods and material are described herein for use in the present disclosure; other, suitable methods and materials known in the art can also be used. The materials, methods, and examples are illustrative only and not intended to be limiting.

Abbreviations used herein and their respective descriptions are listed in Table 1.

Of note, all PD patients were idiopathic PD patients. Thus, both abbreviations “PD” and “iPD” are to be understood as referring to idiopathic PD patients in the context of the illustrative Examples given herein.

Of further note, also the terms “healthy control” (“HC”) and “control” (“CTR”) are used interchangeably herein; and the added terms “_H” and “_L” refer to respective high and low percentiles within a given group, with e.g. “PD_H” referring in the examples herein to the 90th percentile within PD and “PD_L” to the 10th percentile within PD.

TABLE 1

| Abbreviation | Description |
|---|---|
| AAA | Age at assessment |
| AA | Ascorbic acid |
| AAO | Age at onset |
| ATP | Adenosintriphosphate |
| ° C. | Degree Celsius |
| CCCP | Carbonyl cyanide m-chlorophenyl hydrazone |
| cDNA | Complementary DNA |
| CNER | National Committee for Ethics in Research, Luxembourg // Comité |
| | National d'Ethique dans la Recherche |
| CO₂ | Carbon dioxide |
| COURAGE-PD | Comprehensive Unbiased Risk Factor Assessment for Genetics and |
| | Environment in Parkinson's Disease consortium |
| DMEM | Dulbecco's Modified Eagle Medium |
| DNA | Deoxyribonucleic acid |
| DoG | Difference of Gaussian |
| EDTA | Ethylenediaminetetraacetic acid |
| ELISA | Enzyme-linked immunosorbent assay |
| ETC | Electron transport chain |
| FBS | Fetal bovine serum |
| FCCP | Carbonyl cyanide p-trifluoromethoxyphenyl hydrazone |
| FDR | False discover rate |
| FGF | Fibroblast growth factor |
| gDNA | Genomic DNA |
| g/L | Gram per liter |
| GWAS | Genome-wide association study |
| h | Hour |
| HC | Healthy control(s) |
| HI-FBS | Heat-inactivated Fetal Bovine Serum |
| HRP | Horseradish peroxidase |
| IL | Interleukin |
| iPSC | Induced pluripotent stem cell |
| ITS-G | Insulin-Transferrin-Selenium |
| kb | Kilo base pairs |
| LD | Linkage disequilibrium |
| LEDD | L-dopa equivalent daily dose |
| LuxPark | Luxembourg Parkinson's study cohort of the NCER-PD project |
| | (National Centre for Excellence in Research on Parkinson's Disease) |
| MDS-UPDRS | Movement Disorder Society update of the Unified Parkinson's Disease |
| | Rating Scale |
| mito | mitochondrial |
| ml | Milliliter |
| mm | Millimeter |
| mM | Millimolar |
| MoCA | Montreal Cognitive Assessment |
| mt | Mitochondrial |
| μg | Microgram |
| μl | Microliter |
| μM | Micromolar |
| n | Sample size |
| na | Not applicable |
| smNPC | Neuronal progenitor cell |
| ng | Nanogram |
| OCR | Oxygen consumption rate |
| OR | Odds ratio |
| OXPHOS | Oxidative phosphorylation |
| PAGE | Polyacrylamide gel electrophoresis |
| PBS | Phosphate buffered saline |
| PC | Principal component |
| PD | Parkinson's Disease |
| PDQ39 | PD Questionnaire |
| PFA | Paraformaldehyde |
| PMA | Purmorphamine |
| PRS | Polygenic risk score |
| P/S | Penicillin-Streptomycin |
| QC | Quality control |
| r² | Squared coefficient of correlation |
| RNA | Ribonucleic acid |
| ROS | Reactive oxygen species |
| RT-qPCR | Real-time quantitative polymerase chain reaction |
| SDS | Sodium dodecyl sulfate |
| SNP | Single nucleotide polymorphism |
| TCA | Tricarboxylic acid cycle |
| TBS-T | Tris-buffered saline with Tween20 |
| TGF-β1 | Transforming growth factor beta 1 |
| TMRE | Tetramethylrhodamine, Ethyl Ester, Perchlorate |
| v/v | Volume percent |

Materials used are listed in Table 2.

TABLE 2

| Material | Supplier | Cat# |
|---|---|---|
| Antimycin A | | |
| Ascorbic Acid | Sigma | #A4403 |
| B27 | | |
| B27 supplement without | Thermo Fisher Scientific | #12587010 |
| vitamin A | | |
| β-actin primary antibody | Cell signaling | #3700 |
| BSA | | |
| Carbonyl cyanide | Abcam | #ab141229 |
| m-chlorophenyl hydrazone | | |
| Carbonyl cyanide | | |
| p-trifluoromethoxyphenyl | | |
| hydrazone | | |
| CellROXTM Deep Red | Thermo Fisher Scientific, | #C10422 |
| reagent | | |
| CHIR 99021 | Axon Medchem | #2435 |
| Complete Mini EDTA-free | Merck | #11836170001 |
| Proteinase Inhibitor Cocktail | | |
| DMEM | Thermo Fisher Scientific | #21885108 |
| DMEM-F12 // DMEM/F12 | Thermo Fisher Scientific | #21331020 |
| DMEM, high glucose | Thermo Fisher Scientific | #41965039 |
| Dorsomorphin | R&D Systems | #3093 |
| ECLTM Prime Western | Merck | #GERPN2232 |
| Blotting detection reagent | | |
| FBS-deprived DMEM | | |
| FGF-basic | Peprotech | #100-18B |
| Fibroblast medium | | |
| Galactose | | |
| Geltrex | Thermo Fisher Scientific | #A1413202 |
| Glucose | | |
| Glutamax (supplement) | Thermo Fisher Scientific | #35050061 |
| Goat anti-Mouse IgG1 Alexa | Thermo Fisher Scientific | #A21121 |
| Fluor 488 antibody | | |
| Goat serum | | |
| Heparin | Sigma | #H3149-25KU |
| Hepes | Thermo Fisher Scientific | #31330038 |
| HI-FBS | Thermo Fisher Scientific | #10270-106 |
| Hoechst 33342 | Thermo Fisher Scientific | #H21492 |
| Insulin-Transferrin-Selenium | Thermo Fisher Scientific | #41400045 |
| L-Ascorbic acid 2-phosphate | Sigma | #A8960 |
| magnesium | | |
| L-Glutamine | | |
| N2 (supplement) | Thermo Fisher Scientific | #17502001 |
| NaCl | | |
| Neurobasal | Thermo Fisher Scientific | #21103049 |
| Neurobasal-A without | Thermo Fisher Scientific | #A2477501 |
| glucose and sodium pyruvate | | |
| Nonfat-dried milk | | |
| Mitotracker green dye | | |
| Mouse anti-Sox2 antibody | Santa Cruz Biotechnology | #sc-365823 |
| Oligomycin | | |
| Paraformaldehyde | Paraformaldehyde | Paraformaldehyde |
| PBS | | |
| Penicillin-Streptomycin | Thermo Fisher Scientific | #15140163 |
| Peroxidase (HRP)- | | |
| conjugated anti-mouse | | |
| secondary antibody | | |
| Purmorphamine | Sigma | #SML0868 |
| Reprogramming vector hKlf4 | | |
| Reprogramming vector | | |
| hKOS | | |
| Reprogramming vector | | |
| hc-Myc | | |
| RestoreTM PLUS Western | Thermo Fisher Scientific | #46430 |
| Blot Stripping Buffer | | |
| Rotenone | | |
| SB-431542 | R&D Systems | #1614/10 |
| SDS | | |
| Seahorse XF DMEM Medium | Agilent | #103575-100 |
| pH 7.4 | | |
| Sodium pyruvate | | |
| Tetramethylrhodamine, Ethyl | Thermo Fisher Scientific | #T669 |
| Ester, Perchlorate | | |
| TGF-β1 | Peprotech | #100-21 |
| Total OXPHOS-mito Human | | ab110411 |
| WB | | |
| Antibody Cocktail | | |
| Tris-HCl | | |
| Triton-X100 | | |
| Tween 20 | | |
| VECTASHIELD Antifade | Vector Laboratories | H1000 |
| Mounting medium | | |
| XF Calibrant solution | | |

Kits and assays used are listed in Table 3.

TABLE 3

| Kit or Assay | Supplier | Cat# |
|---|---|---|
| CyQUANT assay | | |
| CyQUANT cell proliferation | Thermo Fisher Scientific | #C7026 |
| assay | | |
| Cytoscan HT-CMA 96 array | Thermo Fisher Scientific | |
| CytoTuneTM-iPS 2.0 Sendai | Thermo Fisher Scientific | #A16518 |
| Reprogramming kit | | |
| Genomic DNA Purification kit | Thermo Fisher Scientific | #K0512 |
| High-capacity cDNA Reverse | Thermo Fisher Scientifc | #4368814 |
| Transcription kit | | |
| IL-6 (human) high-sensitive | Enzo | #ENZ-KIT 178-0001 |
| ELISA kit | | |
| LightCycler ® 480 Probes | Roche | #04707494001 |
| Master kit | | |
| PierceTM BCA protein assay | Thermo Fisher Scientific | #23227 |
| QIAmp DNA mini kit | Qiagen | #51304 |
| RNeasy plus mini kit | Qiagen | #74136 |
| Seahorse XFe96 FluxPak kit | Agilent | #102416-100 |
| Taqman probe ACTB-VIC | Thermo Fisher Scientific | Hs03023880_g1 |
| Taqman probe NANOG-FAM | Thermo Fisher Scientific | Hs02387400_g1 |
| Taqman probe OCT3/4-FAM | Thermo Fisher Scientific | Hs00999632_g1 |
| XF Cell Mito Stress Test kit | Agilent | #103015-100 |

Databases and publicly available data sources used are listed in Table 4.

TABLE 4

| Database and/or data | Version and/or source |
|---|---|
| Billingsley I and II | Billingsley et al., 2019 |
| COURAGE-PD (consortium) | COURAGE-PD consortium; Grover et al., |
| | 2021 |
| GWAS summary statistics | Nalls et al., 2019 |
| Haplotype Reference Consortium | r1.1 2016 |
| LuxPark | NCER-PD project; Hipp et al., 2018 |
| MitoCarta3.0 | Rath et al., 2021 |
| Molecular Signatures Database (MsigDB) | v7.5.1; Liberzon, 2014 |

Services used are listed in Table 5.

TABLE 5

| Service | Provider |
|---|---|
| Multiplex qPCR | |
| Real-time single molecule sequencing | PacBio |
| (SMRT) | |
| Sanger sequencing | |

Devices used are listed in Table 6.

TABLE 6

| Device | Supplier |
|---|---|
| 12-well plates | |
| 96-well Seahorse cell culture plates | |
| Cell Carrier Ultra 96-well plates | Perkin Elmer, #6055302 |
| CellVoyager CV8000 High-Content | Yokogawa |
| Screening System | |
| Incubatorr | |
| Neurochip array (v.1.0 and v1.1) | Illumina, San Diego, CA |
| Nitrocellulose membrane | |
| QIAshredder spin columns | Qiagen, #79654 |
| SDS-PAGE (NUPAGETM) | Thermo Fisher Scientific |
| Sensor cartridge | |
| STELLA Bio-imaging system | Raytest Isotopenmessgerate GmbH |
| XFe96 Extracellular flux Analyzer. | Agilent |
| Zeiss spinning disk confocal | Carl Zeiss Microimaging GmbBH |
| microscope | |

Software used is listed in Table 7.

TABLE 7

| Software | Provider | Version |
|---|---|---|
| Matlab | | |
| Michigan Imputation Server | | |
| PLINK | Open source; Chang et al., | v1.9 |
| | 2015 | |
| PRSice2 | | |
| QuantStudio 3D Digital PCR | Applied Biosystems | |
| System | | |
| R | Open source | v4.0.4 |
| Seahorse XF Cell Mito | Agilent | |
| Stress Test Report | | |
| Generator | | |
| XFe Wave software | | |

### Study Design and Workflow

Study design and workflow are schematically depicted in FIG. 1A.

### Investigated Data Sets

Two datasets were analyzed. First, an exploratory dataset from an ongoing Luxembourg Parkinson's, herein referred to also as LuxPark, Study a large longitudinal monocentric observational study in the framework of the NCERPD (National Centre for Excellence in Research in Parkinson's Disease) project, which aimed at recruiting and following up patients with PD and other forms of neurodegenerative parkinsonism along with healthy controls (HC). At the time of data export, the LuxPark study cohort comprised 625 healthy controls (HC) and 493 PD patients. Second, genotyping data from the COURAGE-PD consortium were used for the replication study. Thereof, 24 independent European ancestry cohorts, listed in Table 8, were selected including in total 19,480 individuals (8,626 HC and 10,854 PD patients). More specifically, the replication dataset was used from the COURAGE-PD (Comprehensive Unbiased Risk Factor Assessment for Genetics and Environment in Parkinson's Disease) consortium, including 24 sub-cohorts of European descent, but excluding the Luxembourg Parkinson's Study, thus resulting in the in total 10,854 PD patients and 8,626 HC listed in Table 8. All participants signed a written informed consent and the study was approved by the corresponding national ethics boards.

TABLE 8

| Cohorts | Principal investigators | Country | HC | PD |
|---|---|---|---|---|
| 1 | Aasly | Norway | 509 | 510 |
| 2 | Annesi | Italy | 94 | 93 |
| 3 | Bardien/Carr | South-Africa | 170 | 322 |
| 4 | Brice/Corvol/Lesage | France | 280 | 851 |
| 5 | Carmine Belin/Ran | Sweden | 629 | 286 |
| 6 | Chartier-Harlin/Muttez | France | 228 | 370 |
| 7 | Deutschlander | Germany | 46 | 285 |
| 8 | Elbaz | France | 1,066 | 455 |
| 9 | Farrer | USA | 409 | 389 |
| 10 | Ferreira | Portugal | 67 | 415 |
| 11 | Gasser/Sharma | Germany | 1,070 | 1,664 |
| 12 | Duga/Cilia | Italy | 1,370 | 1,391 |
| 13 | Hadjigeorgiou | Greece | 314 | 283 |
| 14 | Koks/Taba | Estonia | 170 | 216 |
| 15 | Mellick | Australia | 508 | 480 |
| 16 | Pastor/Diez-Fairen | Spain | 338 | 499 |
| 17 | Puschmann | Sweden | 105 | 110 |
| 18 | Rogaeva/Lang | Canada | 153 | 215 |
| 19 | Stefanis/Simitsi | Greece | 181 | 242 |
| 20 | Toft | Norway | 442 | 480 |
| 21 | Tolosa | Spain | 68 | 309 |
| 22 | Valente | Italy | 54 | 326 |
| 23 | Wirdefeldt | Sweden | 171 | 65 |
| 24 | Zimprich | Austria | 184 | 598 |
| Total | | | 8,626 | 10,854 |

For both data sets, LuxPark and COURAGE-PD, DNA samples were genotyped using the Neurochip array that was specifically designed to integrate neurodegenerative disease-related variants (Blauwendraat et al., 2017).

### Variant and Sample Quality-Control

Quality control (QC) of genotyping data from LuxPark was performed using PLINK as follows: samples with call rates <95% and whose genetically determined sex deviated from gender reported in clinical data were excluded from the analysis, and the filtered variants were checked for cryptic relatedness and excess of heterozygosity. Samples exhibiting excess heterozygosity (F statistic >0.2) and first-degree relatedness were excluded. Once sample QC was completed, SNPs with Hardy-Weinberg equilibrium P value <1E-6, and missingness rates >5% were excluded.

QC was conducted independently for each European cohort belonging to the COURAGE-PD consortium, according to standard procedures reported previously (Grover et al., 2021).

For both cohorts, carriers of pathogenic PD-linked variants in eight PD-related genes (ATP13A2, LRRK2, GBA, PARK7, PINK1, PRKN, VPS35 and SNCA) were excluded upon identification using genotyping data. At least for LuxPark, the presence of these variants was confirmed by Sanger sequencing. Moreover, carriers of rare GBA variants in LuxPark were excluded, which were identified by real-time single molecule sequencing and confirmed by Sanger sequencing. To consider the population stratification, five principal components were calculated. Genotyping data were then imputed using the Haplotype Reference Consortium using the Michigan Imputation Server and filtered for imputation quality (R2>0.3) (Das et al., 2016).

### Mitochondrial Gene-Sets and Pathway Resources

To assess potential associations between common variants in nuclear-encoded mitochondrial genes and PD risk, three different mitochondrial gene sets were selected:
i) MitoCarta3.0, which has been the largest public inventory of human genes encoding mitochondrial proteins, more specifically comprising 1,136 genes encoding proteins with anticipated mitochondrial localization,
ii) Two gene sets reported by Billingsley and colleagues: the primary list (Billingsley I) comprising 178 genes implicated in mitochondrial disorders, and the secondary list (Billingsley II) comprising 1,327 genes implicated in regulating, more generally, mitochondrial function, and
iii) Six newly defined groups of genes as shown in Table 9.

As regards iii) above, i.e. the six newly defined groups of genes, the respective genes were manually curated using the Molecular Signatures Database as being known to participate in at least one of the following mitochondrial pathways potentially related to PD pathogenesis: Mitochondrial DNA regulation, Mitophagy, Oxidative phosphorylation (OXPHOS-mito), TCA cycle, Mitochondrial protein import and Mitochondrial proton transport.

TABLE 9

| Gene-sets | Pathways ID | Genes |
|---|---|---|
| Mitophagy | GO:0000423 Mitophagy | AMBRA1, ATG13, ATG14, BECN1, CDC37, CLEC16A, HDAC6, HTRA2, MFN2, |
| (n = 45) | GO:1901524 Regulation of | OPTN, PINK1, PRKN, RNF41, SMURF1, SQSTM1, TIGAR, TOMM7, TSC2, USP30, |
| | mitophagy | VPS13C, VPS13D, ATG12, ATG5, CSNK2A1, CSNK2A2, CSNK2B, FUNDC1, |
| | R-HSA-5205647 Reactome | MAP1LC3A, MAP1LC3B, MFN1, RPS27A, MTERF3, PGAM5, SRC, TOMM20, |
| | mitophagy | TOMM22, TOMM40, TOMM5, TOMM6, TOMM70, UBA52, UBB, UBC, ULK1, VDAC1 |
| TCA cycle | GO:0006099 Tricarboxylic acid | ACO1, ACO2, CS, DHTKD1, DLAT, DLD, DLST, FAHD1, FH, IDH1, IDH2, IDH3A, |
| (n = 177) | cycle | IDH3B, IDH3G, IREB2, MDH1, MDH1B, MDH2, ME2, ME3, NNT, OGDH, OGDHL, |
| | GO:0030062 Mitochondrial | PDHA1, PDHA2, PDHB, SDHA, SDHAF2, SDHB, SDHC, SDHD, SUCLA2, SUCLG1, |
| | tricarboxylic acid cycle enzyme | SUCLG2, BCKDHA, BCKDHB, BCKDK, DBT, MRPS36, KAT2A, ADHFE1, |
| | complex | ATP5F1A, ATP5F1B, ATP5F1C, ATP5F1D, ATP5F1E, ATP5MC1, ATP5ME, |
| | GO:0045239 Tricarboxylic acid | ATP5MF, ATP5MG, ATP5PB, ATP5PD, ATP5PF, ATP5PO, BSG, COX4I1, COX5A, |
| | cycle enzyme complex | COX5B, COX6A1, COX6B1, COX6C, COX7A2L, COX7B, COX7C, COX8A, CYC1, |
| | REACT_111083 Reactome tca | CYCS, D2HGDH, ETFA, ETFB, ETFDH, L2HGDH, LDHA, LDHB, NDUFA1, |
| | cycle and respiratory electron | NDUFA10, NDUFA11, NDUFA12, NDUFA13, NDUFA2, NDUFA3, NDUFA4, |
| | transport | NDUFA5, NDUFA6, NDUFA7, NDUFA8, NDUFA9, NDUFAB1, NDUFB1, NDUFB10, |
| | R-HSA-1428517 Reactome the | NDUFB2, NDUFB3, NDUFB4, NDUFB5, NDUFB6, NDUFB7, NDUFB8, NDUFB9, |
| | citric acid tca cycle and | NDUFC1, NDUFC2, NDUFS1, NDUFS2, NDUFS3, NDUFS4, NDUFS5, NDUFS6, |
| | respiratory electron transport | NDUFS7, NDUFS8, NDUFV1, NDUFV2, NDUFV3, PDHX, PDK1, PDK2, PDK3, |
| | | PDK4, PDP1, PDP2, PDPR, SLC16A1, SLC16A3, SLC16A8, UCP1, UCP2, UCP3, |
| | | UQCR11, UQCRB, UQCRBP1, UQCRC1, UQCRC2, UQCRFS1, UQCRH, UQCRHL, |
| | | UQCRQ, ACAD9, ATP5MC2, ATP5MC3, COQ10A, COQ10B, COX11, COX14, |
| | | COX16, COX18, COX20, GLO1, GSTZ1, HAGH, DMAC2L, ECSIT, LDHAL6A, |
| | | LDHAL6B, ME1, LDHC, LRPPRC, MPC1, MPC2, NDUFAF1, NDUFAF2, NDUFAF3, |
| | | NDUFAF4, NDUFAF5, NDUFAF6, NDUFAF7, NDUFB11, NUBPL, PPARD, RXRA, |
| | | SCO1, SLC25A14, SLC25A27, SURF1, TACO1, TIMMDC1, TMEM126B, TRAP1, |
| | | UQCR10, VDAC1 |
| Oxidative | GO:0006119 Oxidative | ABCD1, ACTN3, AFG1L, ATP5F1A, ATP5F1B, ATP5F1C, ATP5F1D, ATP5F1E, |
| Phoshorylation | phosphorylation | ATP5MC1, ATP5MC2, ATP5MC3, ATP5ME, ATP5MF, ATP5MG, ATP5PB, ATP5PD, |
| (OXPHOS- | GO:0090324 Negative regulation | ATP5PF, ATP5PO, ATP7A, BID, CCNB1, CDK1, CHCHD10, COA6, COQ9, COX10, |
| mito) | of oxidative phosphorylation | COX15, COX4I1, COX4I2, COX5A, COX5B, COX6A1, COX6A2, COX6B1, COX6C, |
| (n = 124) | GO:1903862 Positive regulation | COX7A1, COX7A2, COX7A2L, COX7B, COX7C, COX8A, CYC1, CYCS, DLD, |
| | of oxidative phosphorylation | DMAC2L, DNAJC15, DNAJC30, FXN, MECP2, MLXIPL, MSH2, MYOG, NDUFA1, |
| | | NDUFA10, NDUFA11, NDUFA12, NDUFA13, NDUFA2, NDUFA3, NDUFA4, |
| | | NDUFA5, NDUFA6, NDUFA7, NDUFA8, NDUFA9, NDUFAB1, NDUFAF1, NDUFB1, |
| | | NDUFB10, NDUFB11, NDUFB2, NDUFB3, NDUFB4, NDUFB5, NDUFB6, NDUFB7, |
| | | NDUFB8, NDUFB9, NDUFC1, NDUFC2, NDUFC2-KCTD14, NDUFS1, NDUFS2, |
| | | NDUFS3, NDUFS4, NDUFS5, NDUFS6, NDUFS7, NDUFS8, NDUFV1, NDUFV2, |
| | | NDUFV3, NIPSNAP2, PARK7, PDE12, PGK1, PGK2, PINK1, PPIF, RHOA, SDHA, |
| | | SDHAF2, SDHC, SDHD, SHMT2, SLC25A23, SLC25A33, SNCA, STOML2, SURF1, |
| | | TAZ, TEFM, UQCC2, UQCC3, UQCR10, UQCR11, UQCRB, UQCRC1, UQCRC2, |
| | | UQCRFS1, UQCRH, UQCRHL, UQCRQ, VCP |
| Mitochondrial | GO:0032042 Mitochondrial DNA | CHCHD4, DNA2, FLCN, LIG3, LONP1, MGME1, PARP1, PPARGC1A, SESN2, |
| DNA | metabolic process | STOX1, TOP3A, ATG7, DNAJA3, MIR155, POLG, POLG2, PRIMPOL, RRM2B, |
| (n = 21) | GO:0043504 Mitochondrial DNA | SSBP1, STOML2, TWNK |
| | repair | |
| | GO:0006264 Mitochondrial DNA | |
| | replication | |
| Protein import | GO:0030150 Protein import into | DNAJC15, DNAJC19, DNLZ, GRPEL1, GRPEL2, PAM16, ROMO1, TIMM17A, |
| (n = 19) | mitochondrial matrix | TIMM17B, TIMM21, TIMM23, TIMM23B, TIMM44, TIMM50, TOMM20, TOMM20L, |
| | | TOMM40, TOMM40L, TOMM7 |
| Proton | GO:0033615 mitochondrial proton | ATP23, ATPAF1, FMC1, OXA1L, TMEM70, ATP5MC1, ATP5MC2, ATP5MC3, |
| transport | transporting ATP synthase | ATP5ME, ATP5MF, ATP5MG, ATP5MGL, ATP5PB, ATP5PD, ATP5PF |
| (n = 15) | complex assembly | |
| | GO:0000276 Mitochondrial | |
| | proton-transporting ATP synthase | |
| | complex, coupling factor F(o) | |

### Polygenic Risk Scores (PRSs)

Whole genome PD PRS was calculated using the PRSice2 software under default settings (Choi and O'Reilly, 2019). PRSs for the different mitochondrial gene sets were generated using the PRSet function in PRSice2. For whole genome PRS as well as for mitoPRS, the same approach was applied to general or pathway-specific mitochondrial gene sets. In particular, respective PRSs for each individual were calculated by summing the weighted effects of the risk alleles (minor allele frequency, MAF>1%) associated with PD—based on the largest summary statistics of PD GWAS to date (Nalls et al., 2019)—which were present in the imputed LuxPark and COURAGE-PD genotype data. The criteria for linkage disequilibrium (LD) clumping of SNPs were pairwise LD r²<0.1 within a 250 kb window. PRSs were computed at different P-value thresholds ranging from 5e-08 to 0.5. PRSice2 identified the optimum P-value threshold for variant selection that explains the maximum variance in the target sample. Biospecimens obtained from participants in the Luxembourg Parkinson's Study stratified based on their individual OXPHOS-PRS were used for functional experiments.

### Fibroblasts Selection and Culture

Skin biopsies from the lower back region of both PD patients and healthy controls showing the highest or lowest OXPHOS-mito PRSs (10th vs 90th percentile, n=4 for each group) were collected in low-glucose Dulbecco's Modified Eagle Medium (DMEM) supplemented with 1% (v/v) Penicillin-Streptomycin (P/S). Each skin biopsy (5 mm diameter punch) was cut into up to 5 pieces and placed with the dermis facing down into a cell culture flask containing heat-inactivated Fetal Bovine Serum (HI-FBS). After 10 minutes incubation at room temperature, DMEM containing 4.5 g/L Glucose and 4 mM L-Glutamine, supplemented with 10% (v/V) HI-FBS and 1% (v/v) P/S, was added to the flask, followed by incubation at 37° C. in a 5% CO₂ humidified atmosphere. Cells were kept in the same medium for subsequent subculture, and subjected to Mycoplasma analysis on a regular basis to exclude potential contamination. The 16 fibroblasts lines all had the same passage number at the moment of the experiments, ranging from passage 5 to 10.

Ethical approval for the usage of patient-derived cell lines was given by the National Committee for Ethics in Research, Luxembourg (Comité National d′Ethique dans la Recherche; CNER #201411/05), and all methods were performed in accordance with the relevant guidelines and regulations.

iPSC Generation

Fibroblast reprogramming into induced pluripotent stem cells (iPSCs) was performed by using the CytoTune™-iPS 2.0 Sendai Reprogramming kit, following the manufacturer's instructions (feeder free conditions). Briefly, fibroblasts were transduced overnight with the reprogramming vectors hKOS, hc-Myc and hKlf4, at MOI 5, 5 and 3 respectively, and then maintained in standard fibroblast medium until day 7 post-transduction. On day 7, fibroblasts under reprogramming were plated on Geltrex-coated wells, and switched to iPSCs medium starting from day 8. iPSCs medium consisted in DMEM-F12, Hepes, supplemented with 1% P/S, 1% Insulin-Transferrin-Selenium, 64 μG/mL L-Ascorbic acid 2-phosphate magnesium, 100 ng/ml heparin, 2 ng/ml TGF-β1 and 10 ng/ml FGF-basic. Between day 21 and 28 post-transduction, emerging undifferentiated iPSC colonies were manually transferred into Geltrex-coated 12-well plates for further expansion and characterization.

### Generation and Maintenance of Neuronal Progenitor Cells

The procedure for obtaining neuronal progenitor cells from iPSCs was described previously (Reinhardt et al., 2013), with slight modifications. Briefly, iPSCs were shifted to N2B27 medium-consisting of 49% DMEM/F12, 49% Neurobasal, 1:100 B27 supplement without vitamin A, 1:200 N2 supplement, 1% (v/v) Glutamax and 1% (v/V) P/S-supplemented with 10 μM SB-431542, 1 μM Dorsomorphin, 3 μM CHIR 99021 and 0.5M purmorphamine (PMA). After 4 days in this medium, SB-431542 and Dorsomorphin were removed and cells maintained in N2B27 medium containing CHIR, PMA and 150 μM Ascorbic Acid (AA). On day 5, the emerging neural epithelium was isolated, triturated into smaller pieces and plated on Geltrex-coated wells. The resulting neuroepithelial stem cells (NESCs) were further expanded in N2B27 medium supplemented with CHIR, PMA and AA.

### Analysis of Mitochondrial Respiration

Oxygen consumption rates (OCRs) were determined by using the Seahorse XFe96 FluxPak and XF Cell Mito Stress Test kits, in accordance with manufacturer's instructions. The Seahorse technology was chosen for obtaining a comprehensive overview of mitochondrial bioenergetics by measuring oxygen consumption rates (OCRs). For primary skin fibroblasts, 5,000 cells per well were seeded in a 96-well Seahorse cell culture plate (at least 5 replicates per fibroblast line) and incubated overnight. The following day, fibroblast medium in each well was replaced with 175 μl Seahorse XF DMEM Medium pH 7.4, supplemented with 25 mM Glucose, 2 mM L-Glutamine and 1 mM Sodium Pyruvate, followed by 1h incubation at 37° C. without CO₂. In the meantime, the sensor cartridge—previously equilibrated overnight in the XF Calibrant solution at 37° C. without CO₂—was loaded with standard mitochondrial toxins, namely Oligomycin (1 μM final concentration in the assay well), carbonyl cyanide p-trifluoromethoxyphenyl hydrazone (FCCP, 0.6 μM final concentration in the assay well) and a mixture of Rotenone and Antimycin A (both at the concentration of 0.5 μM in the assay well). After an additional incubation at 37° C. without CO₂ for 30 minutes, the sensor cartridge as well as the cell culture plate were loaded on the XFe96 Extracellular flux Analyzer. Three OCR measurements were performed for both basal respiration and after each automated injection of mitochondrial toxins. Once the run was completed, the cell culture medium was removed from each well and DNA content measured by using the CyQUANT cell proliferation assay kit.

OCR data were analyzed using the XFe Wave software and normalized against cell number, as defined by the CyQUANT assay. Normalized data were finally exported using the Seahorse XF Cell Mito Stress Test Report Generator and statistical analysis performed on at least three independent biological replicates. Initial OCR values as well as OCR measurements after each injection step were used to calculate different parameters related to oxidative phosphorylation, including basal respiration, proton leakage across the inner mitochondrial membrane, respiration coupled to ATP synthesis, maximal respiration and spare respiratory capacity (Gu et al., 2021).

Seahorse experiments in neuronal progenitor cells were performed in Neurobasal medium containing either glucose or galactose as carbon source (Swerdlow et al., 2013). Briefly, 35,000 cells per well were first seeded in a Geltrex-coated 96-well Seahorse cell culture plate (at least 10 replicates per line), in 100 μl N2B27 medium containing CHIR, PMA and AA. The day after, N2B27 medium was removed and replaced by Neurobasal-A without glucose and sodium pyruvate, then supplemented with 0.727 mM sodium pyruvate and either 25 mM Glucose or 25 mM Galactose. Similar to the standard N2B27 used for maintenance of neuronal progenitor cells, this medium also contained N2, B27 and Glutamax supplements, 1% (v/v) P/S, as well as CHIR, PMA and AA. Neuronal progenitor cells remained in glucose- or galactose-based media for 48 hours before proceeding with the Seahorse protocol described above. Either glucose or galactose (always 25 mM) were also added to the Seahorse assay medium (XF DMEM PH 7.4) used during the entire procedure.

### ROS Measurements

Primary skin fibroblasts (about 10,000 cells per well) were seeded into Cell Carrier Ultra 96-well plates. The next day, fibroblasts medium was removed and replaced by fresh medium containing either 5 μM CellROX™ Deep Red reagent or vehicle only (DMSO). 1 μg/ml Hoechst 33342 was added to both solutions to stain the nuclei. After 30 minutes incubation at 37° C. in the dark, cells were washed twice in PBS before proceeding with confocal microscopy analysis. Z-stack images were acquired at 20× magnification using the CellVoyager CV8000 High-Content Screening System.

An in-house Matlab image processing pipeline was used to quantify CellROX signal. The CellROX positive structures where segmented via preprocessing of the CellROX channel via a Difference of Gaussian (DoG) filter with side-length 110 pixels and sigmas of 1 and 33 pixels, prior to thresholding (>10). CellROX total raw intensities within this mask where normalized to nuclear area to extract numeric features for each field of view. Nine fields per well and at least two wells per condition were assessed for each of the three independent biological replicates.

### Analysis of Mitochondrial Morphology and Membrane Potential

Primary skin fibroblasts (10,000 cells per well) were seeded into Cell Carrier Ultra 96-well plates. The next day, fibroblast medium was removed and replaced by FBS-deprived DMEM containing 100 nM Tetramethylrhodamine, Ethyl Ester, Perchlorate (TMRE), a fluorescent dye that specifically accumulates in active, polarized mitochondria, or vehicle only (DMSO). The mitochondrial OXPHOS (OXPHOS-mito) uncoupler carbonyl cyanide m-chlorophenyl hydrazone (CCCP) was used—at the concentration of 50 μM—as a positive control of TMRE staining. Nuclei were stained with 1 μg/ml Hoechst 33342. Cells were incubated in the dark at 37° C. for 30 minutes before proceeding with high-throughput automated confocal microscopy analysis. Z-stack images were acquired at 20× magnification using the CellVoyager CV8000 High-Content Screening System.

An in-house Matlab image processing pipeline was used to quantify TMRE signal. The mitochondria channel was sum-projected on the x-y plane and preprocessed using a DoG filter of 33 pixel side-length, and sigmas set to 1 and 1.5 pixels, prior to thresholding (>10). Connected components with less than 6 pixels were removed from mitochondrial analysis. Total mitochondrial TMRE fluorescence intensity per field was normalized to nuclear area. Morphometric analysis of mitochondrial network was performed as described previously (Antony et al., 2020). Nine fields per well and at least two wells per condition were assessed for each of the three independent biological replicates.

### Analysis of Electron Transport Chain Subunits Expression by Western Blotting

Primary skin fibroblasts were washed twice in Phosphate-Buffered Saline (PBS) and then lysed with 1% sodium dodecyl sulfate (SDS) supplemented with Complete Mini EDTA-free Proteinase Inhibitor Cocktail. Cell lysates were boiled 5 minutes at 95° C., sonicated and protein quantification performed by using the Pierce™ BCA protein assay. 10 μg proteins were first separated by SDS-PAGE and then transferred onto nitrocellulose membrane. The latter was incubated for 1 hour in 5% (w/v) nonfat-dried milk dissolved in TBS-T buffer (10 mM Tris-HCl PH 8.0, 150 mM NaCl and 0.05% Tween 20), followed by overnight incubation at 4° C. with the Total OXPHOS-mito Human WB Antibody Cocktail. The membrane was washed in TBS-T for 30 minutes and then incubated for 1 hour with a peroxidase (HRP)-conjugated anti-mouse secondary antibody. After 30 minutes of washing in TBS-T, the membrane was incubated with the ECL™ Prime Western Blotting detection reagent and chemiluminescent visualization of protein bands performed on the STELLA Bio-imaging system. After removal of conjugated antibodies by means of the Restore™ PLUS Western Blot Stripping Buffer, the same blotting membrane was incubated with B-actin primary antibody, as a control of protein loading.

Analysis mtDNA Copy Number, Transcription/Replication and Deletions

gDNA was extracted from fibroblasts using the QIAmp DNA mini kit following the manufacturer's instructions. Measurement of mtDNA copy number was performed by targeting the mitochondrial gene ND1 and the nuclear single-copy gene B2M using a digital PCR approach (Wasner et al., 2022). MtDNA replication/transcription as well as mtDNA major arc deletions were assessed using previously published methods (He, 2002; Rygiel et al., 2015) by multiplex qPCR.

### Quantification of Plasma IL-6

IL-6 levels were measured in plasma samples from the highest or lowest OXPHOS-mito PRS groups using the IL-6 (human) high-sensitive ELISA kit, following the manufacturer's instructions.

### RT-qPCR

Total RNA was extracted using the RNeasy plus mini kit, after homogenization of the cell lysate by means of the QIAshredder spin columns. 1 μg of RNA was retrotranscribed using the High-capacity cDNA Reverse Transcription kit. The resulting cDNAs were used to quantify the pluripotency markers OCT3/4 and NANOG by multiplex qPCR, using the LightCycler® 480 Probes Master kit combined with the following Taqman probes: OCT3/4-FAM, NANOG-FAM and ACTB-VIC.

### Immunocytochemistry

iPSCs were seeded in 24-well plates containing Geltrex-coated glass coverslips. Three days after seeding, iPSC medium was removed, followed by two washes in PBS and fixation in 4% paraformaldehyde (PFA). After 15 minutes incubation, PFA was removed and the coverslips washed twice in PBS before proceeding with the standard immunofluorescence protocol. Briefly, cells were incubated for 1 hour in permeabilization and blocking solution (i.e. PBS containing 0.4% Triton-X100, 10% normal goat serum and 2% BSA), followed by two washes in PBS. Cells were then incubated overnight at 4° C. with mouse anti-Sox2 primary antibody, diluted 1:250 in PBS containing 0.1% Triton-X100, 1% normal goat serum and 0.2% BSA. The following day, cells were washed three times in PBS and then incubated for 2 hours at room temperature with Goat anti-Mouse IgG1 Alexa Fluor 488 secondary antibody, diluted 1:1000 in PBS containing 0.1% Triton-X100, 1% normal goat serum and 0.2% BSA. After three washes in PBS, nuclei were stained with 1 μg/ml Hoechst 33342 and glass coverslips finally mounted onto the slide in presence of 10 μl VECTASHIELD Antifade Mounting medium. Images were acquired using a Zeiss spinning disk confocal microscope.

### Karyotype and Cell Identity Analysis

Genomic DNA (gDNA) was purified from frozen iPSCs pellets according to the Genomic DNA Purification kit. 100 ng total gDNA was used to prepare the Cytoscan HT-CMA 96 array for KaryoStat+ analysis, based on copy number variants (CNV) and single nucleotide polymorphisms (SNP) optimized for balanced whole-genome coverage. The KaryoStat+ assay enabled the detection of chromosomal aberrations, including aneuploidies, submicroscopic aberrations and mosaic events, with a resolution similar to g-banding karyotyping (>1 Mb for chromosomal gains and losses). Using the same array as the KaryoStat assay, the cell identity (Cell ID) assay allowed for DNA fingerprint matching between newly-generated iPSCs lines and the original fibroblast lines, through correlation analysis of 150k SNP probes across the genome of both cell types. This approach enabled detection of unique DNA based signatures of the genetic background of a cell, which were used for comparative analysis.

### Clinical Outcomes

In the LuxPark cohort, both PD patients and HC underwent a comprehensive assessment of clinical phenotype, including both motor and non-motor symptoms. Focus was put on eight PD-specific clinical outcomes, namely the Movement Disorder Society update of the Unified Parkinson's Disease Rating Scale I (MDS-UPDRS I), MDS-UPDRS II, MDS-UPDRS III, MDS-UPDRS-IV, the PD Quality of Life Questionnaire (PDQ39), the L-dopa equivalent daily dose (LEDD), the Scales for Outcomes in Parkinson's Disease-Autonomic Dysfunction (SCOPA-AUT) and the Montreal Cognitive Assessment (MoCA). Age at onset (AAO) of PD symptoms and disease duration were also assessed. Age at onset (AAO) was set as age at PD diagnosis and the disease duration was calculated based on AAO to the time of clinical assessment or time to skin biopsy respectively.

### Statistical Analysis

PRS distribution in HC vs iPD subjects were compared using the non-parametric Wilcoxon rank-sum test. The predictive accuracy of the PRS model was determined using the area under the receiver operating curve (AUC, pROC R package). A higher AUC indicated a better PRS model that can differentiate between PD cases and controls. To further examine if PRS might predict risk of PD, a logistic regression model was used to calculate the odds ratio (OR). Gender, age at assessment (AAA) and the first three PCs from the population stratification were included as covariates. Individuals were stratified in three groups based on OXPHOS-mito PRS percentiles: low (<10%), intermediate (10-90%) and high (>90%) PRS. Comparison of PD-specific clinical outcomes, age at onset (AAO) and disease duration between individuals in the high and low OXPHOS-mito PRS groups was performed using the Wald test. Statistical analyses were done in R and p-values adjusted using False Discovery Rate (FDR<0.05) correction (Benjamini-Hochberg method) for the number of independent tests. Statistical analyses of functional data were performed using the GraphPad Prism software (v9.4.0).

Association of Common Variants in Mitochondrial Genes with PD Risk

To gain essential knowledge on the mitochondria-related “missing heritability” in idiopathic PD, the distribution of mitochondria-specific polygenic risk scores (mito PRSs) was analyzed in two independent patient cohorts, namely LuxPark (the Luxembourg Parkinson's Study) and COURAGE-PD (Comprehensive Unbiased Risk factor Assessment for Genetics and Environment in Parkinson's Disease).

### The LuxPark Dataset

After filtering and quality control (QC), the final LuxPark dataset for the subsequent analyses comprised 412 PD patients and 576 healthy controls (HC). PD patients were older than the HC (67.5±10.9 vs 59.1±12.2 years, p<0.01) with mean age at onset (AAO) of 62.2±11.8 years and mean disease duration of 5.4±5.0 years (Table 10). Using common SNP data (minor allele frequency, MAF>1%) from the latest PD GWAS for the LuxPark cohort. The genome-wide PRS was significantly associated with PD compared to HC (OR=1.57 per one standard deviation; FDR adjusted p-value: FDR-adj p=1.28e-098; FIG. 2A).

TABLE 10
Demographic data of iPD patients and PD-linked mutation-free HC from the
Luxembourg Parkinson's study (LuxPark) and Courage-PD cohorts. Means
and percentage were calculated after genotyping data quality controls.

| | Luxembourg Parkinson's Study | | Courage-PD | |
|---|---|---|---|---|
| | PD | HC | PD | HC |
| n | 412 | 576 | 10449 | 8484 |
| Age at assessment (mean ± SD) | 67.5 ± 10.9 | 59.1 ± 12.2 | 67.1 ± 10.9 | 65.5 ± 11.6 |
| Age at onset (mean ± SD) | 62.2 ± 11.8 | — | 58.7 ± 11.4 | — |
| Sex (% male/female) | 67.9/32.1 | 56.9/43.1 | 60.2/39.8 | 46.1/53.9 |
| Disease duration (mean ± SD) | 5.4 ± 5.0 | — | 8.3 ± 6.5 | — |

Methodologic Validation—Whole Genome mitoPRS

To capture the cumulative effect of common variants in nuclear-encoded mitochondrial genes on PD risk, three different mitochondrial gene sets were selected: Human MitoCarta3.0, a public inventory of 1,136 genes encoding proteins with anticipated mitochondrial localization and two additional gene lists previously used for mitoPRSs calculation by Billingsley et al, 2019 containing genes implicated in mitochondrial disorders (Billingsley I, 178 genes) and regulating mitochondria function (Billingsley II, 1327 genes), respectively. Of note, all three mitochondrial gene-sets tested were significantly associated with PD (FDR adjusted p-value <0.05, FIG. 2 B): (i) Billingsley I (OR=1.18 [1.0-1.4]), (ii) Billingsley II (OR=1.26 [1.0-1.5]) and (iii) MitoCarta3.0 (OR=1.24 [1.0-1.4]). The degree of overlap between the three mitochondrial gene sets is shown in FIG. 1 B.

### Pathway Specific PRS

The PRS approach, first for applied to the whole genome, was then applied to distinct mitochondrial pathways to obtain mitoPRSs related to specific mitochondrial alterations possibly involved in PD pathogenesis. Therefore, six mitochondrial pathways potentially relevant in PD were selected with each pathway being represented by a group of genes (Table 9) annotated in the corresponding Molecular Signatures Database (MsigDB) sub-collection, and mitoPRSs calculated. As shown in FIG. 2 C, only variants in the oxidative phosphorylation (OXPHOS-mito) gene set were significantly associated with a higher PD risk in the LuxPark cohort (OR=1.31 [1.1-1.5], p=5.4e-04).

To evaluate mitochondria-specific genetic risk without considering the potential contribution of variants in known PD loci included in the OXPHOS-mito gene list (i.e. PINK1, SNCA and DJ-1), the PRS analysis was also performed for the OXPHOS-mito gene group after exclusion of the known PD-related genes PINK1, SNCA and DJ-1. In this setting, the OR was slightly reduced (OR=1.24 [1.1-1.4], p=5.3e-03), but the association with PD risk was nevertheless significant (FIG. 2 D).

### COURAGE-PD as Replication Dataset

To validate these findings, the COURAGE-PD consortium was used as a larger replication dataset. After filtering and QC, the COURAGE-PD dataset included 10,449 PD cases and 8,484 HC from 24 European sub-cohorts (Table 8). Genome-wide PRS as well as all pathway specific mitoPRSs tested were significantly associated with PD risk in COURAGE-PD (FIG. 2A to D). In particular, OXPHOS-mito (OR=1.21 [1.18-1.26], p=3.4e-36) and TCA cycle (OR=1.22 [1.18-1.26], p=1.5e-36) were the pathways showing the highest association with PD risk based on the respective pathway specific mitoPRSs (FIG. 2 C). Again, OXPHOS-mito was significantly associated with PD even after exclusion of the PD-related genes PINK1, SNCA and DJ-1 (OR=1.14 [1.11-1.18], p=3.4e-36) (FIG. 2 D). A subset of 137 genes of the OXPHOS-mito gene group, not associated with TCA cycle, was also found to be statistically significant associated with PD risk as well.

### Functional Validation of Predicted Phenotypes in Patient-Based Cellular Models Using Primary Skin Fibroblasts Based on OXPHOS-Mito PRS

To functionally validate the association between i) common variants in genes regulating oxidative phosphorylation and ii) increased PD risk, mitochondrial respiration was assessed in primary skin fibroblasts from both HC and PD patients stratified based on their individual OXPHOS-mito PRS. Therefore, two groups of PD patients showing the highest or lowest OXPHOS-mito PRSs (10th and 90th percentile, respectively) were identified in the LuxPark cohort and primary skin fibroblasts selected for functional characterization (n=4 per group). Following the same approach, fibroblasts from HC with extreme OXPHOS-mito PRS values (10th and 90th percentile, n=4 per group) were investigated as a reference for mitochondrial readouts from cells derived from PD patients (FIG. 3A).

Gender, age of HC and PD patients at onset of first symptoms and at the time of skin biopsy, respectively, are shown in FIG. 3 B and indicated in Table 11.

TABLE 11

| | | Fibroblast | | Age at | Age at |
|---|---|---|---|---|---|
| Status | PRS | ID | Gender | onset | biopsy |
| HC | LOW_OXPHOS- | #18486 | M | na | 68 |
| | mito | #18490 | F | na | 63 |
| | | #18617 | M | na | 77 |
| | | #26871 | F | na | 60 |
| | HIGH_OXPHOS- | #10365 | M | na | 63 |
| | mito | #11020 | M | na | 71 |
| | | #15818 | M | na | 72 |
| | | #37562 | M | na | 48 |
| PD | LOW_OXPHOS- | #17162 | M | 66 | 69 |
| | mito | #18250 | M | 57 | 66 |
| | | #18263 | M | 52 | 55 |
| | | #21990 | M | 70 | 71 |
| | HIGH_OXPHOS- | #11043 | M | 68 | 71 |
| | mito | #12251 | M | 50 | 51 |
| | | #15092 | M | 66 | 67 |
| | | #15809 | F | 40 | 44 |

Elevated Mitochondrial Oxygen Consumption in Primary Skin Fibroblasts from PD Patients with High OXPHOS-Mito PRS

The Seahorse technology was used to analyze respiratory chain performance over a standard mitochondrial stress test assay, by measuring oxygen consumption rates (OCRs) under basal conditions and after targeted inhibition of specific respiratory chain complexes (RCC).All parameters analyzed-including basal respiration, proton leakage, ATP-linked respiration, maximal respiration, spare respiratory capacity and non-mitochondrial respiration—did not significantly vary between HC with highest or lowest OXPHOS-mito PRSs. Likewise, OCRs measured in fibroblasts from PD patients exhibiting low OXPHOS-mito PRSs were not significantly different from those observed in HC cells. Interestingly, In particular, basal respiration, proton leak and ATP-linked respiration were all significantly enhanced in fibroblasts from iPD patients with high OXPHOS-PRS compared to low OXPHOS-PRS cells, reaching OCR levels well beyond the reference threshold defined by fibroblasts of healthy individuals. In this setting, maximal respiration was not significantly different between high and low OXPHOS-PRS iPD fibroblasts, thus indicating a reduced mitochondrial reserve capacity in the high OXPHOS-PRS iPD group (FIGS. 4 and 5). Of note, all respiratory parameters analyzed did not significantly vary between HC with high or low OXPHOS-PRS (FIGS. 4 and 5). Likewise, biogenesis of respiratory chain complexes (RCC) did not differ significantly between high and low OXPHOS-PRS groups—both in HC and in iPD patients—as revealed by immunoblot analysis of RCC protein subunits (FIG. 6).

Collectively, these findings indicated that fibroblasts of PD patients with high OXPHOS-mito PRS displayed features of mitochondrial hyperactivity already under steady-state conditions; a phenotype that appeared to be independent of RCC expression levels.

Other Functional Readouts of Mitochondrial Activity Remained Unchanged in Fibroblasts from PD Patients Stratified Based on OXPHOS-Mito PRS

To assess whether changes in OCR were accompanied by differences in reactive oxygen species (ROS) production as a major byproduct of oxidative metabolism and potential cause of neurodegeneration, primary skin fibroblasts from both controls and PD patients stratified based on their individual OXPHOS-mito PRS were subjected to CellROX staining as oxidative stress readout. Surprisingly, increased ROS levels were detected in HC from the high OXPHOS-mito PRS group, but not in PD patients using one-way ANOVA (FIG. 7A and FIG. 8A). This finding appeared to argue against a causal link between elevated oxidative phosphorylation and ROS accumulation in PD.

Mitochondrial membrane potential (Δψm) was also measured in primary skin fibroblasts from the OXPHOS-mito PRS group. As expected, FCCP-treated cells failed to sequester the dye, and displayed a robust reduction of TMRE fluorescence. However, no significant differences in Δψm were observed in untreated fibroblasts from both HC and PD patients previously selected based on low or high OXPHOS-mito PRS (FIG. 7 B and FIG. 8 B).

Furthermore, mitochondrial network morphology was analyzed in primary skin fibroblasts from low vs high OXPHOS-mito PRS HC and PD groups, after live imaging staining with Mitotracker green dye. Again, no significant difference in any of the morphological parameters assessed was observed, including mitochondrial aspect ratio and form factor (FIG. 7 C and FIG. 8 C).

Mitochondrial DNA (mtDNA) copy number as well as mtDNA transcription/replication, which could impinge on respiratory chain activity by altering the expression and stoichiometric assembly of mtDNA-encoded electron transport chain (ETC) complex subunits, were investigated, but did not reveal any significant change between the high and low OXPHOS-mito PRS groups, both in HC and in PD fibroblasts (FIGS. 9A and B). Similarly, no significant variation in mtDNA deletion levels among different groups was observed (FIG. 9 C).

Investigation of Blood Plasma from OXPHOS-Mito PRS Stratified HC and PD Patients

Interleukin-6 (IL-6) levels were measured in view of a potential link between mitochondrial dysfunction and inflammation in blood plasma samples from LuxPark study participants—both HC and PD patients—with the highest or lowest OXPHOS-mito PRS. However, no significant difference in plasma IL-6 levels between high and low OXPHOS-mito PRS groups was detected (FIG. 7 D).

Altogether, findings obtained both in primary skin fibroblasts and in blood plasma samples from LuxPark study participants suggested that altered mitochondrial respiration observed in PD patients stratified based on OXPHOS-mito PRS was specifically related to the cumulative effect of common variants in nuclear-encoded mitochondrial genes regulating oxidative phosphorylation, and not an indirect consequence of other mitochondrial activities like ROS production, mitochondrial membrane potential, mitochondrial network morphology and mitochondrial ETC complex subunit expression.

Functional Validation of OXPHOS-Mito PRS in iPSC-Derived Neuronal Cells from PD Patients

It was examined, whether altered mitochondrial respiration observed in primary skin fibroblasts from high-risk PD patients could be also detected in patient-derived neuronal models. Therefore, fibroblast lines were reprogrammed into induced pluripotent stem cells (iPSCs). Given that fibroblasts from HC did not display major differences in oxidative phosphorylation when comparing low- and high-risk groups (FIGS. 4 and 5), only PD fibroblast lines were investigated (10th vs 90th OXPHOS-mito PRS percentile, n=4 per group). Among these, 2 lines per group were successfully reprogrammed into iPSC (#17162 and #18250 for the low OXPHOS-mito PRS; #11043 and #15092 for the high OXPHOS-mito PRS) without displaying any chromosomal aberration and derived from male individuals as expected (Table 11, FIG. 10A). Correlation analysis of 150k SNPs spread across the genome of iPSC clones vs the original skin fibroblasts confirmed identical genetic background between the two cell types (FIG. 10 B). Different from fibroblasts, all iPSC lines expressed high RNA levels of the stemness markers OCT3/4 and NANOG (FIG. 10 C). Expression of the stem cell marker SOX2 was confirmed by immunofluorescence (FIG. 10 D). After molecular characterization and QC, the in total 4 successfully reprogrammed iPSC lines were differentiated into neuronal progenitor cells (smNPCs), and subjected to mitochondrial respiration analyses.

Contrary to observations in primary skin fibroblasts, Seahorse-based assessment of oxygen consumption rates in smNPCs did not reveal significant differences between low and high OXPHOS-mito PRS groups (FIG. 11). This finding was likely related to high glycolytic activity and low reliance on OXPHOS-mito metabolism of neural stem cells. In accordance with this hypothesis, the shift from glucose to galactose medium as carbon source significantly increased basal and ATP-linked respiration of smNPCs, indicative of a metabolic switch from glycolysis to oxidative phosphorylation (FIG. 11). In this setting, smNPCs derived from PD patients showing high OXPHOS-mito PRS displayed a significant decrease in different oxygen consumption rates (OCR) measured, including basal, maximal and ATP-linked respiration (FIG. 11). Thus, when forced to use oxidative phosphorylation, smNPCs derived from PD patients with high OXPHOS-mito PRS displayed a significant reduction in their oxygen consumption rates.

Thus, findings in iPSC-derived neuronal progenitor cells from PD patients classified at risk for genetic variants in OXPHOS-mito related loci, effectively confirmed an impairment of mitochondrial respiration.

An exciting aspect of our findings is that iPD patients with high OXPHOS-PRS display an earlier age at PD onset (AAO) compared to low-risk patients, a clinical phenotype typically observed in familial, monogenic PD forms (e.g., PINK1-PD). We therefore extended our functional analysis to cell lines from two distinct PD patients carrying the Q456X mutation in the PINK1 gene, and assessed mitochondrial respiration in the corresponding neuronal progenitor cells (smNPCs). We previously showed that, when forced to use oxidative phosphorylation (OXPHOS) to produce ATP (i.e., in galactose medium), smNPCs derived from iPD patients with high OXPHOS-PRSs displayed a significant decrease of basal and ATP-linked respiration compared to the low OXPHOS-PRSs group (FIG. 11). We have now demonstrated, in the same setting, that smNPCs with high OXPHOS-PRS display a level of impairment in mitochondrial respiration comparable to that observed in PINK1-mutant smNPCs (FIG. 15). This further reinforces the effectiveness of our functionally validated genetic stratification approach, and highlights the possibility to include sporadic PD patients with high OXPHOS-PRS in future clinical trials with mitochondrially active drugs.

Association of OXPHOS-Mito PRSs with PD-Specific Clinical Outcomes

After functional validation of the OXPHOS-mito PRS approach, mean of age-at-onset (AAO), disease duration and eight PD-specific motor and non-motor clinical scores were compared between highest or lowest (10th and 90th percentile, respectively) OXPHOS-mito PRSs and whole-genome PRS in LuxPark PD and COURAGE-PD patients. As shown in FIG. 12, individuals with high OXPHOS-PRS tended to have earlier AAO and longer disease duration compared to low-risk patients, a phenotype particularly evident in the large COURAGE-PD dataset (p<0.05; AAO, p=0.0036; disease duration, p=0.033). Analyses were extended to eight PD-specific motor and non-motor clinical scores that were only available for the Luxembourg Parkinson's Study. The whole-genome PRS was used as reference. Individuals with high OXPHOS-PRS showed worse quality of life (as defined by higher PDQ-39), but none of these scores reached statistical significance (FIG. 13). In the extended group of clinical outcomes, the trend of clinical outcomes analyzed, including Montreal Cognitive Assessment (MoCa), Scales for Outcomes in Parkinson's Disease-Autonomic Dysfunction (SCOPA-AUT) and MDS-Unified Parkinson's Disease Rating Scale (UPDRS) scores, was similar to that observed in the whole-genome PRS group.

Embodiments illustratively described herein may suitably be practiced in the absence of any element or elements, limitation or limitations, not specifically disclosed herein. Thus, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group.

Equivalents: Those skilled in the art will recognize or be able to ascertain using no more than routine experimentation, many equivalents to the specific embodiments of the invention described herein. Such equivalents are intended to be encompassed by the following claims.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

Further embodiments will become apparent from the appended claims.

### CITED REFERENCES

Antony, P. M. A. et al., 2020. Fibroblast mitochondria in idiopathic Parkinson's disease display morphological changes and enhanced resistance to depolarization. Sci Rep 10, 1569. https://doi.org/10.1038/s41598-020-58505-6
Billingsley, K. J. et al., 2019. Mitochondria function associated genes contribute to Parkinson's Disease risk and later age at onset. npj Parkinsons Dis. 5, 8. https://doi.org/10.1038/s41531-019-0080-x
Blauwendraat, C. et al., 2017. NeuroChip, an updated version of the NeuroX genotyping platform to rapidly screen for variants associated with neurological diseases. Neurobiology of Aging 57, 247.e9-247.e13. https://doi.org/10.1016/j.neurobiolaging.2017.05.009
Chang, C. C. et al., 2015. Second-generation PLINK: rising to the challenge of larger and richer datasets. GigaSci 4, 7. https://doi.org/10.1186/s13742-015-0047-8
Choi, S.W., and O'Reilly, P. F., 2019. PRSice-2: Polygenic Risk Score software for biobank-scale data. GigaScience 8, giz082. https://doi.org/10.1093/gigascience/giz082
Das, S. et al., 2016. Next-generation genotype imputation service and methods. Nat Genet 48, 1284-1287. https://doi.org/10.1038/ng.3656
Grover, S. et al., COURAGE-PD Consortium, 2021. Replication of a Novel Parkinson's Locus in a European Ancestry Population. Mov Disord 36, 1689-1695. https://doi.org/10.1002/mds.28546
Gu, X. et al, 2021. Measurement of mitochondrial respiration in adherent cells by Seahorse XF96 Cell Mito Stress Test. STAR Protocols 2, 100245. https://doi.org/10.1016/j.xpro.2020.100245
He, L., 2002. Detection and quantification of mitochondrial DNA deletions in individual cells by real-time PCR. Nucleic Acids Research 30, 68e-668. https://doi.org/10.1093/nar/gnf067
Hipp, G. et al., 2018. The Luxembourg Parkinson's Study: A Comprehensive Approach for Stratification and Neurosci. 10, 326. Early Diagnosis. Front. Aging https://doi.org/10.3389/fnagi.2018.00326
Liberzon, A., 2014. A Description of the Molecular Signatures Database (MSigDB) Web Site, in: Kidder, B. L. (Ed.), Stem Cell Transcriptional Networks, Methods in Molecular Biology. Springer New York, New York, NY, pp. 153-160. https://doi.org/10.1007/978-1-4939-0512-6_9
Nalls, M.A. et al., 2019. Identification of novel risk loci, causal insights, and heritable risk for Parkinson's disease: a meta-analysis of genome-wide association studies. The Lancet Neurology 18, 1091-1102. https://doi.org/10.1016/S1474-4422(19)30320-5
Rath, S. et al., 2021. MitoCarta3.0: an updated mitochondrial proteome now with sub-organelle localization and pathway annotations. Nucleic Acids Research 49, D1541-D1547. https://doi.org/10.1093/nar/gkaa1011
Reinhardt, P. et al., 2013. Derivation and Expansion Using Only Small Molecules of Human Neural Progenitors for Neurodegenerative Disease Modeling. PLOS ONE 8, e59252. https://doi.org/10.1371/journal.pone.0059252
Rygiel, K. A. et al., 2015. Triplex real-time PCR—an improved method to detect a wide spectrum of mitochondrial DNA deletions in single cells. Sci Rep 5, 9906. https://doi.org/10.1038/srep09906
Swerdlow, R. H. et al., 2013. Glycolysis-respiration relationships in a neuroblastoma cell line. Biochimica et Biophysica Acta (BBA)—General Subjects s 1830, 2891-2898. https://doi.org/10.1016/j.bbagen.2013.01.002
Wasner, K. et al., 2022. Parkin Deficiency Impairs Mitochondrial DNA Dynamics and Propagates Inflammation. Movement Disorders mds.29025. https://doi.org/10.1002/mds.29025

1. A method for stratifying whether or not a subject may be at a risk of suffering from Parkinson's disease (PD), comprising determining in a sample obtained from said subject its OXPHOS-mito polygenic risk score, wherein a high OXPHOS-mito polygenic risk score is indicative that said subject may be at a risk of developing PD.

2. A method for stratifying whether or not a subject may be at a risk of suffering from Parkinson's disease (PD), comprising determining in a sample obtained from said subject its OXPHOS-mito polygenic risk score, wherein a low OXPHOS-mito polygenic risk score is indicative that said subject may not be at a risk of developing PD.

3. A method for stratifying whether or not a subject suffering from Parkinson's disease (PD) may be at a risk of having mitochondrial dysfunction, comprising determining in a sample obtained from said subject its OXPHOS-mito polygenic risk score (PRS), wherein a high OXPHOS-mito PRS is indicative that said subject may be at a risk of developing mitochondrial dysfunction and/or wherein a low OXPHOS-mito PRS is indicative that said subject may not be at a risk of developing mitochondrial dysfunction.

4. The method according to claim 3, wherein the mitochondrial dysfunction is characterized by increased mitochondrial oxygen consumption or decreased mitochondrial oxygen consumption.

5. A method for stratifying whether or not a subject may be at risk suffering from Parkinson's disease (PD) with an early age of onset, comprising determining in a sample obtained from said subject its OXPHOS-mito polygenic risk score (PRS), wherein a high OXPHOS-mito PRS is indicative that said subject may be at a risk of developing Parkinson's disease (PD) with an early age of onset and/or wherein a low OXPHOS-mito PRS is indicative that said subject may not be at a risk of developing Parkinson's disease (PD) with an early age of onset.

6. A compound for use in a method for preventing and/or treating Parkinson's disease in a subject, wherein said subject has a high OXPHOS-mito polygenic risk score.

7. The compound for the use of claim 6, wherein said compound modulates mitochondrial oxygen consumption.

8. The compound for the use of claim 6 or 7, wherein modulation of mitochondrial oxygen consumption is an increase of mitochondrial oxygen consumption or a decrease of mitochondrial oxygen consumption.

9. The compound for the use of any one of claims 6-8, wherein the compound is a drug that targets one or more electron transport chain proteins, wherein the one or more electron transport chain proteins are preferably selected from the group consisting of complexes I, II, III, and IV, and ATP synthase.

10. The compound for the use of any one of claims 6-9, wherein the compound is selected from the group consisting of Nobiletin, NAHS, Compound A and rT1.

11. The compound for the use of any one of claims 6-10, wherein the compound is coenzyme Q10.

12. Use of a cell being characterized by a high OXPHOS-mito polygenic risk score for screening compounds for the prevention or treatment of Parkinson's disease.

13. The use of claim 6, wherein said compound modulates mitochondrial oxygen consumption.

14. The use of claim 6 or 7, wherein modulation of mitochondrial oxygen consumption is an increase of mitochondrial oxygen consumption or a decrease of mitochondrial oxygen consumption.

15. The use of any one of claims 6 to 8, wherein said cell is a fibroblast, an induced pluripotent stem cell (iPSC) or induced pluripotent stem cell (iPSC)-derived neuronal progenitor cell (smNPC).

16. A method for screening compounds for the prevention or treatment of Parkinson's disease, comprising bringing a cell being characterized by a high OXPHOS-mito polygenic risk score into contact with said compound and determining whether said compound modulates mitochondrial oxygen consumption of said cell.

17. The method of claim 16, wherein modulation of mitochondrial oxygen consumption is an increase of mitochondrial oxygen consumption or a decrease of mitochondrial oxygen consumption.

18. The method of claim 16 or 17, wherein said cell is a fibroblast, an induced pluripotent stem cell (iPSC) or induced pluripotent stem cell (iPSC)-derived neuronal progenitor cell (smNPC).

19. A composition comprising a cell being characterized by a high OXPHOS-mito polygenic risk score, optionally further comprising a buffer.

20. The composition of claim 19, wherein said cell is a fibroblast, an induced pluripotent stem cell (iPSC) or induced pluripotent stem cell (iPSC)-derived neuronal progenitor cell (smNPC).

21. The composition of claim 19 or 20, further comprising a compound that modulates mitochondrial oxygen consumption of said cell.

22. A system comprising a cell being characterized by a high OXPHOS-mito polygenic risk score, optionally further comprising a buffer.

23. The system of claim 22, wherein said system is adapted for screening compounds which decreases/increases mitochondrial oxygen consumption of said cell.

24. The system of claim 22 or 23, wherein said cell is a fibroblast, an induced pluripotent stem cell (iPSC) or induced pluripotent stem cell (iPSC)-derived neuronal progenitor cell.

25. The system of any one of claims 22-24, further comprising a compound that modulates mitochondrial oxygen consumption of said cell, wherein the iPSC-derived neuronal progenitor cell is preferably a neuronal progenitor cell (smNPC).

26. A compound obtained or obtainable by the method of any one of claims 16 to 18.

27. A method for stratifying whether or not a subject suffering from PD may be eligible for a treatment with a compound according to any one of claims 6-11 and 26, comprising determining in a sample obtained from said subject its OXPHOS-mito polygenic risk score (PRS), wherein a high OXPHOS-mito PRS is indicative that said subject may be eligible for treatment with said compound and/or wherein a low OXPHOS-mito PRS is indicative that said subject may not be eligible for treatment with said compound.

28. The method of any one of claims 1-5 and 27, the compound for the use of any one of claims 6-11, the use of any one of claims 12-15, the method of any one of claims 16-18, the composition of any one of claims 19-21, the system of any one of claims 22-25, and/or the compound of claim 26, wherein Parkinson's disease is idiopathic Parkinson's disease.

29. The method of any one of claims 1-5 and 27-28, the compound for the use of any one of claims 6-11 and 28, the use of any one of claims 12-15 and 28, the method of any one of claims 16-18 and 28, the composition of any one of claims 19-21 and 28, the system of any one of claims 22-25 and 28, and/or the compound of any one of claims 26 and 28, wherein Parkinson's disease is Parkinson's disease is associated with mitochondrial dysfunction and/or aberrant mitochondrial oxygen consumption.

30. The method of any one of claims 1-5 and 27-29, the compound for the use of any one of claims 6-11 and 28-29, the use of any one of claims 12-15 and 28-29, the method of any one of claims 16-18 and 28-29, the composition of any one of claims 19-21 and 28-29, the system of any one of claims 22-25 and 28-29, and/or the compound of any one of claims 26 and 28-29, wherein the OXPHOS-mito polygenic risk score is obtained for a gene set comprising at least about 50, at least about 60, at least about 70, at least about 80, at least about 90, at least about 100, at least about 110, at least about 115, at least about 120, such as at least about 121, at least about 122, at least about 123, or about 124 of the OXPHOS-mito genes listed in Table 9.

31. The method of any one of claims 1-5 and 27-30, the compound for the use of any one of claims 6-11 and 28-30, the use of any one of claims 12-15 and 28-30, the method of any one of claims 16-18 and 28-30, the composition of any one of claims 19-21 and 28-30, the system of any one of claims 22-25 and 28-30, and/or the compound of any one of claims 26 and 28-30, wherein the OXPHOS-mito polygenic risk score is obtained based on the OXPHOS-mito genes listed in Table 9.

32. The method of any one of claims 1-5 and 27-31, the compound for the use of any one of claims 6-11 and 28-31, the use of any one of claims 12-15 and 28-31, the method of any one of claims 16-18 and 28-31, the composition of any one of claims 19-21 and 28-31, the system of any one of claims 22-25 and 28-31, and/or the compound of any one of claims 26 and 28-31, wherein the OXPHOS-mito polygenic risk score is obtained for a gene set comprising at least about 50, at least about 60, at least about 70, at least about 80, at least about 90, at least about 100, at least about 110, at least about 115, at least about 120, such as at least about 121, at least about 122, at least about 123, or about 124 human genes having the Gene Ontology Database Pathways IDs of GO:0006119 (Oxidative phosphorylation), GO:0090324 (Negative regulation of oxidative phosphorylation), and GO:1903862 (Positive regulation of oxidative phosphorylation).

33. The method of any one of claims 1-5 and 27-32, the compound for the use of any one of claims 6-11 and 28-32, the use of any one of claims 12-15 and 28-32, the method of any one of claims 16-18 and 28-32, the composition of any one of claims 19-21 and 28-32, the system of any one of claims 22-25 and 28-32, and/or the compound of any one of claims 26 and 28-32, wherein the OXPHOS-mito polygenic risk score is obtained for a gene set comprising at least about 50, at least about 60, at least about 70, at least about 80, at least about 90, at least about 100, at least about 110, at least about 115, at least about 120, such as at least about 121, at least about 122, at least about 123, or about 124 genes selected from the group consisting of ABCD1, ACTN3, AFG1L, ATP5F1A, ATP5F1B, ATP5F1C, ATP5F1D, ATP5F1E, ATP5MC1, ATP5MC2, ATP5MC3, ATP5ME, ATP5MF, ATP5MG, ATP5PB, ATP5PD, ATP5PF, ATP5PO, ATP7A, BID, CCNB1, CDK1, CHCHD10, COA6, COQ9, COX10, COX15, COX411, COX412, COX5A, COX5B, COX6A1, COX6A2, COX6B1, COX6C, COX7A1, COX7A2, COX7A2L, COX7B, COX7C, COX8A, CYC1, CYCS, DLD, DMAC2L, DNAJC15, DNAJC30, FXN, MECP2, MLXIPL, MSH2, MYOG, NDUFA1, NDUFA10, NDUFA11, NDUFA12, NDUFA13, NDUFA2, NDUFA3, NDUFA4, NDUFA5, NDUFA6, NDUFA7, NDUFA8, NDUFA9, NDUFAB1, NDUFAF1, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFC2-KCTD14, NDUFS1, NDUFS2, NDUFS3, NDUFS4, NDUFS5, NDUFS6, NDUFS7, NDUFS8, NDUFV1, NDUFV2, NDUFV3, NIPSNAP2, PARK7, PDE12, PGK1, PGK2, PINK1, PPIF, RHOA, SDHA, SDHAF2, SDHC, SDHD, SHMT2, SLC25A23, SLC25A33, SNCA, STOML2, SURF1, TAZ, TEFM, UQCC2, UQCC3, UQCR10, UQCR11, UQCRB, UQCRC1, UQCRC2, UQCRFS1, UQCRH, UQCRHL, UQCRQ, and VCP.

34. The method of any one of claims 1-5 and 27-33, the compound for the use of any one of claims 6-11 and 28-33, the use of any one of claims 12-15 and 28-33, the method of any one of claims 16-18 and 28-33, the composition of any one of claims 19-21 and 28-33, the system of any one of claims 22-25 and 28-33, and/or the compound of any one of claims 26 and 28-33, wherein the OXPHOS-mito polygenic risk score is obtained using GWAS data for Parkinson's Disease.

35. The method of any one of claims 1-5 and 27-34, the compound for the use of any one of claims 6-11 and 28-34, the use of any one of claims 12-15 and 28-34, the method of any one of claims 16-18 and 28-34, the composition of any one of claims 19-21 and 28-34, the system of any one of claims 22-25 and 28-34, and/or the compound of any one of claims 26 and 28-34, wherein the OXPHOS-mito polygenic risk score is obtained using GWAS data as published by Nalls et al. Lancet Neurol. 2019 December; 18(12): 1091-1102 doi: 10.1016/S1474-4422(19)30320-5.

36. The method of any one of claims 1-5 and 27-35, the compound for the use of any one of claims 6-11 and 28-35, the use of any one of claims 12-15 and 28-35, the method of any one of claims 16-18 and 28-35, the composition of any one of claims 19-21 and 28-35, the system of any one of claims 22-25 and 28-35, and/or the compound of any one of claims 26 and 28-35, wherein the OXPHOS-mito polygenic risk score is obtained using GWAS data from the LuxPark dataset (Hipp et al., 2018 Front. Aging Neurosci. 10, 326. https://doi.org/10.3389/fnagi.2018.00326).

37. The method of any one of claims 1-5 and 27-36, the compound for the use of any one of claims 6-11 and 28-36, the use of any one of claims 12-15 and 28-36, the method of any one of claims 16-18 and 28-36, the composition of any one of claims 19-21 and 28-36, the system of any one of claims 22-25 and 28-36, and/or the compound of any one of claims 26 and 28-36, wherein the OXPHOS-mito polygenic risk score is obtained using GWAS data from the COURAGE-PD dataset (Grover et al., 2021, Mov Disord 36, 1689-1695. https://doi.org/10.1002/mds.28546).


SEQUENCE LISTING (1 sequence)

SEQ ID NO: 1 - protein, 27 aa, synthetic construct
CIPRGGICLVALSGCCNSPGCIFGICA